(19) United States
(12) Patent Application Publication
(10) Pub. No.: US 2025/0054624 A1
(43) Pub. Date: Feb. 13, 2025

(54) METHODS AND SYSTEMS FOR DIGITAL PATHOLOGY ASSESSMENT OF CANCER VIA DEEP LEARNING

(71) Applicant: Artera Inc., Mountain View, CA (US)

(72) Inventors: Andre Esteva, Mountain View, CA (US); Felix Feng, Hillsborough, CA (US)

(21) Appl. No.: 18/680,486

(22) Filed: May 31, 2024

Related U.S. Application Data
(60) Provisional application No. 63/470,505, filed on Jun. 2, 2023.

(51) Int. Cl. G16H 50/20; G16H 10/40; G16H 10/60; G16H 30/40; G16H 50/70

(52) U.S. Cl. CPC G16H 50/20; G16H 10/40; G16H 10/60; G16H 30/40; G16H 50/70

(57) ABSTRACT

The present disclosure provides methods and systems for classifying and/or monitoring a cancer of a subject. A method for assessing a cancer of a subject may comprise obtaining a data set comprising image and/or tabular data from the subject and processing the data with one or more trained algorithms to classify the cancer of the subject. The cancer of the subject may be assessed based on the results of the classification.

### CROSS-REFERENCE

This application claims the benefit of Provisional Application No. 63/470,505, filed Jun. 2, 2023, which is incorporated by reference herein in its entirety.

### BACKGROUND

Prostate cancer is a leading cause of cancer death in men. Nevertheless, international standards for prognostication of patient outcomes are reliant on non-specific and insensitive tools that commonly lead to over- and under-treatment.

### SUMMARY

Determining a patient's optimal cancer therapy is a challenging task, in which oncologists must choose a therapy with the highest likelihood of success and the least likelihood of toxicity. The difficulties in therapy selection are rooted in the vast molecular, phenotypic, and prognostic heterogeneity exhibited by cancer. Recognized herein is a need for accurate, globally scalable tools to support personalizing cancer therapy.

The present disclosure provides methods and systems for identifying or monitoring cancer-related states by processing biological samples obtained from or derived from subjects, e.g., a cancer patient. Biological samples (e.g., tissue samples) obtained from subjects may be analyzed to prognose clinical outcomes (which may include, e.g., distant metastasis, biochemical recurrence, death, progression free survival, and overall survival).

In an aspect, the present disclosure provide for a method for assessing a cancer of a subject, comprising: (a) obtaining a dataset comprising image data and tabular data derived from the subject; (b) processing the dataset using a trained algorithm to classify the dataset to a category among a plurality of categories, wherein the classifying comprises applying an image processing algorithm to the image data; and (c) assessing the cancer of the subject based at least in part on the category among the plurality of categories that is classified in (b).

In some embodiments, the trained algorithm is trained using self-supervised learning. In some embodiments, the trained algorithm comprises a deep learning algorithm. In some embodiments, the trained algorithm comprises a first trained algorithm processing the image data and a second trained algorithm processing the tabular data. In some embodiments, the trained algorithm further comprises a third trained algorithm processing outputs of the first and second trained algorithms. In some embodiments, the cancer is bladder cancer, breast cancer, cervical cancer, colorectal cancer, gastric cancer, kidney cancer, liver cancer, ovarian cancer, pancreatic cancer, prostate cancer, or thyroid cancer. In some embodiments, the cancer is prostate cancer. In some embodiments, the tabular data comprises clinical data of the subject. In some embodiments, the clinical data of the subject comprises laboratory data, therapeutic interventions, or long-term outcomes. In some embodiments, the image data comprises digital histopathology data. In some embodiments, the histopathology data comprises images derived from a biopsy sample of the subject. In some embodiments, the images are acquired via microscopy of the biopsy sample. In some embodiments, the digital histopathology data is derived from the subject prior to the subject receiving a treatment. In some embodiments, the treatment comprises radiotherapy (RT). In some embodiments, the RT comprises pre-specified use of short-term androgen deprivation therapy (ST-ADT), long-term ADT (LT-ADT), dose escalated RT (DE-RT), or any combination thereof. In some embodiments, the digital histopathology data is derived from the subject subsequent to the subject receiving a treatment. In some embodiments, the treatment comprises radiotherapy (RT). In some embodiments, the RT comprises pre-specified use of short-term androgen deprivation therapy (ST-ADT), long-term ADT (LT-ADT), dose escalated RT (DE-RT), or any combination thereof. In some embodiments, the method further comprises processing the image data using an image segmentation, image concatenation, object detection algorithm, or any combination thereof. In some embodiments, the method further comprises extracting a feature from the image data.

In another aspect, the present disclosure provides for a method for assessing a cancer of a subject, comprising: (a) obtaining a dataset comprising at least image data derived from the subject; (b) processing the dataset using a trained algorithm to classify the dataset to a category among a plurality of categories, wherein the classifying comprises applying an image processing algorithm to the image data, wherein the trained algorithm is trained using self-supervised learning; and (c) assessing the cancer of the subject based at least in part on the category among the plurality of categories that is classified in (b).

In some embodiments, the trained algorithm comprises a deep learning algorithm. In some embodiments, the cancer is bladder cancer, breast cancer, cervical cancer, colorectal cancer, gastric cancer, kidney cancer, liver cancer, ovarian cancer, pancreatic cancer, prostate cancer, or thyroid cancer. In some embodiments, the cancer is prostate cancer. In some embodiments, the image data comprises digital histopathology data. In some embodiments, the histopathology data comprises images derived from a biopsy sample of the subject. In some embodiments, the images are acquired via microscopy of the biopsy sample. In some embodiments, the digital histopathology data is derived from the subject prior to the subject receiving a treatment. In some embodiments, the treatment comprises radiotherapy (RT). In some embodiments, the RT comprises pre-specified use of short-term androgen deprivation therapy (ST-ADT), long-term ADT (LT-ADT), dose escalated RT (DE-RT), or any combination thereof. In some embodiments, the digital histopathology data is derived from the subject subsequent to the subject receiving a treatment. In some embodiments, the treatment comprises radiotherapy (RT). In some embodiments, the RT comprises pre-specified use of short-term androgen deprivation therapy (ST-ADT), long-term ADT (LT-ADT), dose escalated RT (DE-RT), or any combination thereof. In some embodiments, the method further comprises processing the image data using an image segmentation, image concatenation, or object detection algorithm. In some embodiments, the method further comprises extracting a feature from the image data. In some embodiments, the dataset comprises image data and tabular data. In some embodiments, the trained algorithm comprises a first trained algorithm processing the image data and a second trained algorithm processing the tabular data. In some embodiments, the trained algorithm further comprises a third trained algorithm processing outputs of the first and second trained algorithms. In some embodiments, the tabular data comprises clinical data of the subject. In some embodiments, the clinical data comprises laboratory data, therapeutic interventions, or long-term outcomes.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a computer system that is programmed or otherwise configured to implement methods provided herein.

FIGS. 2A-2C show an example of a multimodal deep learning system and dataset. FIG. 2A shows that the multi-modal architecture comprises three parts: a tower stack to parse the tabular clinical data, a tower stack to parse a variable number of digital histopathology slides, and a third tower stack to merge the resultant features and predict binary outcomes. FIG. 2B shows the training of the self-supervised model of the image tower stack. FIG. 2C shows the first five columns of the table show the statistics from each trial. The column ‘combined’ shows the statistics of the final dataset with all five trials used for training and validation. ***RTOG 9413 randomized patients in a 2×2 fashion testing the effect of timing of hormone therapy (before vs. starting with RT) and field size (prostate only vs. full pelvic RT). New acronyms utilized: disease-free survival (DFS), progression-free survival (PFS), prostate cancer-specific mortality (PCSM).

FIGS. 3A-3H show an example of a comparison of the deep learning system to established clinical guidelines across trials and outcomes. FIG. 3A shows performance results reporting on the area under the curve (AUC) of sensitivity and specificity of the MMAI (blue bars) vs NCCN (gray bars) models, using time-dependent receiver operator characteristics. Comparison is made across 5- and 10-year timepoints on the following binary outcomes: distant metastasis (DM), biochemical recurrence (BCR), prostate cancer-specific survival (PCaSS), and overall survival (OS). FIG. 3B shows a summary table of the relative improvement of the AI model over the NCCN model across the various outcomes broken down by performance on the data from each trial in the test set. Relative improvement is given by (PAI−PNCCN)/PNCCN, where P is the performance of a model. FIG. 3C shows the results of an ablation study showing model performance when trained on a sequentially decreasing set of data inputs. NCCN means the following three variables: combined Gleason, baseline psa, t-stage; NCCN+3 means NCCN plus: Gleason primary, Gleason secondary, age; path refers to digitized histopathology images. FIGS. 3D-3H show a performance comparison on the individual clinical trial subsets of the test set—together, these five comprise the entire test set shown in FIG. 3A.

FIG. 4 shows an example of pathologist interpretation of SSL tissue clusters. The self-supervised model in the multi-modal model is trained to identify whether or not augmented versions of small patches of tissue come from the same original patch, without ever seeing clinical data labels. After training, each image patch in the dataset of 10.05M image patches is fed through this model to extract a 128-dimensional feature vector, and the UMAP algorithm31 is used to cluster and visualize the resultant vectors. A pathologist is then asked to interpret the 20 image patches closest to each of the 25 cluster centroids—the descriptions are shown next to the insets. For clarity, we only highlight 6 clusters (colored), and show the remaining clusters in gray. See FIG. 7 for full pathologist annotation.

FIG. 5 shows an example of image quilts for four example patients. The dataset contains patients with a variable number of histopathology slides. To standardize the image inputs to the model, the tissue from each slide is segmented, and all tissues are pasted into a single square image of 51200×51200 pixels and divided into 200 by 200 patches, representing all the histopathology data of a single patient. Image quilts from four patients are shown here.

FIG. 6 shows an example of nucleic density sampling of example image patches. Tan brown boxes indicate nuclei detection, which is used for calculating nucleic density. We oversample the patches that are inputted to the self-supervised training protocol according to nucleic density. Each patch is binned into deciles according to density, and each decile is oversampled such that the MMAI model sees the same number of total images from each decile.

FIG. 7 shows an example of pathologist-interpreted patch clusters. Using UMAP, 25 clusters are generated from the SSL features of all the histopathology patches of trial RTOG-9202. Each row in the image corresponds to the 25 nearest-neighbor image patches of the cluster centroid. These have been inspected by a pathologist to determine the human-interpretable descriptions of the clusters listed in the table.

FIG. 8 shows an example of an NCCN model algorithm. A rule-based algorithm modeling the National Cancer Center Network's annual published guidelines, based on the D'Amico risk groups.

FIG. 9 is a schematic representation of a multi modal AI system as described herein.

FIG. 10 is a flow diagram representing clinical trial pooling for testing and development of models described herein.

FIG. 11 is a table summarizing patient characteristics of data analyzed by models as described herein.

FIG. 12 depicts distributions for MMAI scores as determined by an MMAI model described herein for distant metastasis (DM) and prostate cancer-specific morality (PCSM) between racial subgroups in test (top panel) and development (bottom panel) cohorts.

FIG. 13 is a table showing MMAI scores summarized by racial subgroups on development and test cohorts.

FIG. 14 shows MMAI model scores as determined by MMAI models described herein summarized by racial subgroups in training and test cohorts.

FIGS. 15A-15D show subdistribution hazard ratio (HR) results from Fine & Gray regression models in racial subgroups for distant metastasis (DM) MMAI and prostate cancer-specific morality (PCSM) MMAI in the development and test cohorts. FIG. 15A shows the DM results for the test cohort. FIG. 15B shows the DM results for the development cohort. FIG. 15C shows the PCSM results for the test cohort. FIG. 15D shows the DM results for the development cohort.

FIG. 16 depicts subdistribution hazard ratio (HR) results from Fine & Gray regression models in racial subgroups for a MMAI model as described herein. Shown are HRs for 5-year biochemical failure (BF5yr MMAI), 10-year BF (BF10yr MMAI), 5-year distant metastasis (DM5yr MMAI), 10-year DM (DM10yr MMAI), 10-year prostate cancer-specific mortality (PCSM10yr MMAI), and 10-year overall survival (OS10yr MMAI) in the test cohort

FIG. 17 depicts subdistribution hazard ratio (HR) results from Fine & Gray regression models in racial subgroups for a MMAI model as described herein for DM 5-yr MMAI (panel a) and PCSM 10-yr MMAI (panel b) in tabular form the test and training cohorts.

FIGS. 18A and 18B show estimate risks/cumulative incidence curves by racial subgroups for DM (FIG. 18A) and PCSM (FIG. 18B) in the full cohort.

FIG. 19A shows risk stratifications of MMAI models within racial subgroups (DM MMAI) in the development, test, and full cohorts.

FIG. 19B shows risk stratifications of MMAI models within racial subgroups (PCSM) MMAI in the development, test, and full cohorts.

FIG. 20A shows risk stratifications of MMAI models within racial subgroups (DM MMAI) in the development, test, and full cohorts.

FIG. 20B shows risk stratifications of MMAI models within racial subgroups (PCSM) MMAI in the development, test, and full cohorts.

FIG. 21 shows cumulative incidence curves for distant metastasis (DM) in a cohort of prostate cancer patients.

FIG. 22 is a table summarizing patient characteristics of cohorts stratified by risk as predicted by an artificial intelligence model described herein.

FIG. 23 is a table showing differential risk stratification of the same patient cohort by National Comprehensive Cancer Network (NCCN) risk stratification and by multi-modal artificial intelligence risk stratification.

FIG. 24 shows MMAI-predicted risk of distant metastasis after ten years (DM 10-yr) for a cohort of patients as compared to NCCN classification.

FIGS. 25A and 25B show diagrammatic representations of the differential stratification of a patient cohort by NCCN methods and methods disclosed herein.

FIG. 26 is a flow diagram of patients toward study inclusion from parent clinical trial NRG/RTOG 9902. H&E=hematoxylin and eosin; MMAI=multi-modal artificial intelligence; DPEP=digital pathological evaluable population; RT=radiation therapy; AS=androgen suppression; CT=chemotherapy

FIG. 27A shows populations characteristics for participants from parent clinical trial NRG/RTOG 9902. FIG. 27B shows MMAI scores between treatment arms of the population of individual sin NRG/RTOG 9902.

FIG. 28A is a table showing univariable analysis of association between MMAI algorithms and DM and PCSM endpoints. FIG. 28B is a table showing multivariable analysis of association between MMAI algorithms and DM and PCSM endpoints while adjusting individual clinical risk factors.

FIG. 29A is a table showing prognostic performance of MMAIs for distant metastasis (DM). FIG. 29B is a table showing prognostic performance of MMAIs for prostate cancer specific mortality (PCSM) within subgroup classifications.

FIG. 30A is a table showing multivariable analysis of MMAI algorithms on PM after adjusting for all clinical risk factors. FIG. 30B is a table showing multivariable analysis of MMAI algorithms on PCSM after adjusting for all clinical risk factors.

FIG. 31A is a table showing multivariable analysis of DM-prognostic MMAI algorithms on BF, CSM, and OS. FIG. 31B is a table showing multivariable analysis of PCSM-prognostic MMAI algorithms on BF, CSM, and OS.

FIG. 32A depicts a cumulative incidence curve for estimated distant metastasis (DM) risk by quartile 4 vs. quartile 1-3 as precited by a multimodal artificial intelligence optimized for DM (DM MMAI). FIG. 32B depicts a cumulative incidence curve for estimated prostate cancer specific mortality risk (PCSM) by quartile 4 vs. quartile 1-3 as predicted by a multimodal artificial intelligence optimized for PCSM (PCSM MMAI).

FIG. 33A depicts a cumulative incidence curve for estimated distant metastasis (DM) risk by quartile 4 vs. quartile 1-3 DM MMAI by treatment arm. FIG. 33B depicts a cumulative incidence curve for estimated prostate cancer specific mortality risk (PCSM) by quartile 4 vs. quartile 1-3 PCSM MMAI by treatment arm.

FIG. 34 depicts a non-limiting example of a cohort selection diagram for the analysis cohort, wherein APA denotes apalutamide, and H&E denotes hematoxylin & eosin.

FIGS. 35A-35B depict MFS by treatment group hazard ratio (HR) for apalutamide compared to placebo. FIG. 35A depicts an MMAI non-high risk group (HR: 0.59, 95% CI:0.32-1.09, p=0.09), and FIG. 35B depicts an MMAI high risk group (HR: 0.19, 95% CI: 0.12-0.29, p<0.005), wherein MFS denotes metastasis-free survival, and MMAI denotes Multimodal Artificial Intelligence.

FIGS. 36A-36B depict PFS2 by treatment group hazard ratio (HR) for apalutamide compared to placebo for MMAI non-high risk group (HR: 0.76, 95% CI: 0.45-1.28, p=0.30) (FIG. 36A) and MMAI high risk group (HR: 0.47, 95% CI: 0.33-0.68, p<0.005) (FIG. 36B), wherein PFS2 denotes progression-free survival, and MMAI denotes Multimodal Artificial Intelligence.

FIGS. 37A-37B depict OS by treatment group hazard ratio (HR) for apalutamide compared to placebo. FIG. 37A depicts an MMAI non-high risk group (HR: 0.99, 95% CI: 0.55-1.76, p=0.97), and FIG. 37B depicts an MMAI high risk group (HR: 0.6, 95% CI: 0.40-0.89, p=0.01), wherein OS denotes overall survival, and MMAI denotes Multimodal Artificial Intelligence.

FIG. 38 depicts a non-limiting example of a schematic overview of the multimodal artificial intelligence (MMAI) architecture, wherein PSA denotes prostate-specific antigen, SSL denotes self-supervised learning, MIL denotes multiple instance learning, and frozen denotes a SSL encoder that was locked/not altered.

FIGS. 39A-39C depict overall analysis cohort outcomes by MMAI risk grouped by hazard ratio (HR) for high compared to non-high MFS (HR: 1.47, 95% CI: 1.03, 2.11, p=0.04) (FIG. 39A), PFS2 (HR: 1.30, 95% CI: 0.96, 1.76, p=0.09) (FIG. 39B), and OS (1.15, 95% CI: 0.82, 1.61, p=0.42) (FIG. 39C), wherein MFS denotes metastasis-free survival, PFS2 denotes progression-free survival, OS denotes overall survival, and MMAI denotes Multimodal Artificial Intelligence.

FIGS. 40A-40C depict placebo cohort outcomes by MMAI risk grouped by hazard ratio (HR) for high compared to non-high MFS HR of 2.98 (95% CI: 1.72-5.18, p<0.005) (FIG. 40A), PFS2 HR of 1.83 (95% CI: 1.09-3.09, p=0.02) (FIG. 40B), and OS HR of 1.56 (95% CI: 0.89-2.75, p=0.12) (FIG. 40C), wherein MFS denotes metastasis-free survival, PFS2 denotes progression-free survival, OS denotes overall survival, and MMAI denotes Multimodal Artificial Intelligence.

FIGS. 41A-41C depict Apalutamide cohort outcomes by MMAI risk grouped by hazard ratio (HR) for high compared to non-high MFS HR of 0.92 (95% CI: 0.56-1.51, p=0.75) (FIG. 41A), PFS2 HR of 1.13 (95% CI: 0.77-1.66, p=0.53) (FIG. 41B), and OS HR of 0.96 (95% CI: 0.63-1.47, p=0.85) (FIG. 41C), wherein MFS denotes metastasis-free survival, PFS2 denotes progression-free survival, OS denotes overall survival, and MMAI denotes Multimodal Artificial Intelligence.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, an individual, or a patient. A subject can be a vertebrate, such as, for example, a mammal. Non-limiting examples of mammals include humans, simians, farm animals, sport animals, rodents, and pets. A subject can be a male subject. A subject can be a female subject. The subject may be displaying a symptom(s) indicative of a health or physiological state or condition of the subject, such as a cancer-related health or physiological state or condition of the subject. As an alternative, the subject can be asymptomatic with respect to such health or physiological state or condition. The subject may be suspected of having a health or physiological state or condition. The subject may be at risk of developing a health or physiological state or condition. The health or physiological state may correspond to a disease (e.g., cancer). The subject may be an individual diagnosed with a disease. The subject may be an individual at risk of developing a disease.

As used herein, “diagnosis of cancer,” “diagnosing cancer,” and related or derived terms include the identification of cancer in a subject, determining the malignancy of the cancer, or determining the stage of the cancer.

As used herein, “prognosis of cancer,” “prognosing cancer,” and related or derived terms include predicting the clinical outcome of the patient, assessing the risk of cancer recurrence, determining treatment modality, or determining treatment efficacy.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

Despite the prevalence of prostate cancer, accurate, sensitive, and specific diagnosis of prostate cancer remains elusive. While prostate cancer is often indolent, and treatment can be curative, prostate cancer represents the leading global cause of cancer-associated disability due to the negative effects of over- and under-treatment and remains one of the leading causes of cancer death in men. Determining the optimal course of therapy for patients with prostate cancer is a difficult medical task that involves considering the patient's overall health, the characteristics of their cancer, the side effect profiles of many possible treatments, outcomes data from clinical trials involving patients with similar diagnoses, and prognosticating the expected future outcomes of the patient at hand. This challenge is compounded by the lack of readily accessible prognostic tools to better risk stratify patients.

Artificial intelligence (AI) has permitted insights to be gleaned from massive datasets that had previously resisted interpretation. Whereas standard risk-stratification tools are fixed and based on few variables, AI can learn from large amounts of minimally processed data across various modalities. AI systems may be low-cost, massively scalable, and incrementally improve through usage.

There is a great need for accurate, globally scalable tools to support personalizing therapy. Methods and systems as disclosed herein demonstrate prostate cancer therapy personalization by predicting long-term, clinically relevant outcomes (e.g., distant metastasis, biochemical recurrence, partial response, complete response, death, relative survival, cancer-specific survival, progression free survival, disease free survival, five-year survival, and overall survival) using a novel multimodal deep learning model trained on digital histopathology of prostate biopsies and clinical data.

The present disclosure provides methods, systems, and kits for identifying or monitoring cancer-related categories and/or states by processing biological samples obtained from or derived from subjects (e.g., male patients suffering from or suspected of suffering from prostate cancer). Biological samples (e.g., prostate biopsy samples) obtained from subjects may be analyzed to identify the cancer-related category (which may include, e.g., measuring a presence, absence, or quantitative assessment (e.g., risk, predicted outcome) of the cancer-related category). Such subjects may include subjects with one or more cancer-related categories and subjects without cancer-related categories. Cancer-related categories or states may include, for example, positive for a cancer, negative for a cancer, cancer stage, predicted response to a cancer treatment, and/or predicted long-term outcome (e.g., disease metastasis, biochemical recurrence, partial response, complete response, relative survival, cancer-specific survival, progression free survival, disease free survival, five-year survival, or overall survival).

### Assaying Biological Samples

A biological sample may be obtained or derived from a human subject (e.g., a male subject). The biological sample may be stored in a variety of storage conditions before processing, such as different temperatures (e.g., at room temperature, under refrigeration or freezer conditions, at 25° C., at 4° C., at −18° C., −20° C., or at −80° C.), different suspensions (e.g., formalin, EDTA collection tubes, cell-free RNA collection tubes, or cell-free DNA collection tubes), or. The biological sample may be obtained from a subject having or suspected of having cancer (e.g., prostate cancer), or from a subject that does not have or is not suspected of having cancer.

A biological sample may be used for diagnosing, detecting, or identifying a disease or health or physiological condition of a subject by analyzing the biological sample. The biological sample or part thereof may be analyzed to determine a likelihood the sample is positive for a disease or health condition (e.g., prostate cancer). Alternatively, or additionally, methods as described herein may include diagnosing a subject with the disease or health condition, monitoring the disease or health condition in the subject, and/or determining a propensity of the subject for the health disease/condition. In some embodiments, the biological sample(s) may be used to classify the sample and/or subject into a cancer-related category and/or identify the subject as having a particular cancer-related state. The cancer-related category or state may comprise a diagnosis (e.g., positive or negative for cancer), a particular type of cancer (e.g., prostate cancer), a stage of cancer, a predicted outcome or prognosis, a predicted response to a treatment or treatments, or any combination thereof.

Any substance that is measurable may be the source of a sample. The substance may be a fluid, e.g., a biological fluid. A fluidic substance may include blood (e.g., whole blood, plasma, serum), cord blood, saliva, urine, sweat, serum, semen, vaginal fluid, gastric and digestive fluid, cerebrospinal fluid, placental fluid, cavity fluid, ocular fluid, serum, breast milk, lymphatic fluid, or combinations thereof.

The substance may be solid, for example, a biological tissue. The substance may comprise normal healthy tissues. The tissues may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

The substance may comprise a tumor. Tumors may be benign (non-cancer), pre-malignant, or malignant (cancer), or any metastases thereof. Non-limiting examples of tumors and associated cancers may include: acoustic neuroma, acute lymphoblastic leukemia, acute myeloid leukemia, adenocarcinoma, adrenocortical carcinoma, AIDS-related cancers, AIDS-related lymphoma, anal cancer, angiosarcoma, appendix cancer, astrocytoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, bronchogenic carcinoma, cerebellar astrocytoma, cervical cancer, childhood cancers, chondrosarcoma, chordoma, choriocarcinoma, chronic lymphocytic leukemia, chronic myelogenous leukemia, chronic myeloproliferative disorders, colon cancer, colon carcinoma, craniopharyngioma, cutaneous T-cell lymphoma, cystadenocarcinoma, desmoplastic small round cell tumor, embryonal carcinoma, endocrine system carcinomas, endometrial cancer, endotheliosarcoma, ependymoma, epithelial carcinoma, esophageal cancer, Ewing's sarcoma, fibrosarcoma, germ cell tumors, gallbladder cancer, gastric cancer, gastrointestinal carcinoid tumor, gastrointestinal stromal tumor, gastrointestinal system carcinomas, genitourinary system carcinomas, gliomas, hairy cell leukemia, head and neck cancer, heart cancer, hemangioblastoma, hepatocellular (liver) cancer, Hodgkin lymphoma, Hypopharyngeal cancer, intraocular melanoma, islet cell carcinoma, Kaposi sarcoma, kidney cancer, laryngeal cancer, leiomyosarcoma, lip and oral cavity cancer, liposarcoma, liver cancer, lung cancers, such as non-small cell and small cell lung cancer, lung carcinoma, lymphangiosarcoma, lymphangioendotheliosarcoma, lymphomas, leukemias, macroglobulinemia, malignant fibrous histiocytoma of bone/osteosarcoma, medulloblastoma, medullary carcinoma, melanomas, meningioma, mesothelioma, metastatic squamous neck cancer with occult primary, mouth cancer, multiple endocrine neoplasia syndrome, myelodysplastic syndromes, myeloid leukemia, myxosarcoma, nasal cavity and paranasal sinus cancer, nasopharyngeal carcinoma, neuroblastoma, non-Hodgkin lymphoma, non-small cell lung cancer, oligodendroma, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, papillary adenocarcinoma, papillary carcinoma, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, sebaceous gland carcinoma, seminoma, skin cancers, skin carcinoma merkel cell, small intestine cancer, soft tissue sarcoma, squamous cell carcinoma, stomach cancer, sweat gland carcinoma, synovioma, T-cell lymphoma, testicular tumor, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, Wilms tumor, or combinations thereof. The tumors may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof.

The substances may comprise a mix of normal healthy tissues or tumor tissues. The tissues may be associated with various types of organs. Non-limiting examples of organs may include brain, breast, liver, lung, kidney, prostate, ovary, spleen, lymph node (including tonsil), thyroid, pancreas, heart, skeletal muscle, intestine, larynx, esophagus, stomach, or combinations thereof. In some embodiments, the tissues are associated with a prostate of the subject. In the case of a biological sample comprising cells and/or tissue (e.g., a biopsy sample), the biological sample may be further analyzed or assayed. In some embodiments, the biopsy sample may be fixed, processed (e.g., dehydrated), embedded, frozen, stained, and/or examined under a microscope. In some embodiments, digital slides are generated from processed samples.

In some embodiments, the substance may comprise a variety of cells, including eukaryotic cells, prokaryotic cells, fungi cells, heart cells, lung cells, kidney cells, liver cells, pancreas cells, reproductive cells, stem cells, induced pluripotent stem cells, gastrointestinal cells, blood cells, cancer cells, bacterial cells, bacterial cells isolated from a human microbiome sample, and circulating cells in the human blood. In some embodiments, the substance may comprise contents of a cell, such as, for example, the contents of a single cell or the contents of multiple cells.

In some embodiments, the substances may comprise one or more markers whose presence or absence is indicative of some phenomenon such as disease, disorder, infection, or environmental exposure. A marker can be, for example, a cell, a small molecule, a macromolecule, a protein, a glycoprotein, a carbohydrate, a sugar, a polypeptide, a nucleic acid (e.g., deoxyribonucleic acid (DNA), ribonucleic acid (RNA)), a cell-free nucleic acid (e.g., cf-DNA, cf-RNA), a lipid, a cellular component, or combinations thereof.

The biological sample may be taken before and/or after treatment of a subject with cancer. Biological samples may be obtained from a subject during a treatment or a treatment regimen. Multiple biological samples may be obtained from a subject to monitor the effects of the treatment over time. The biological sample may be taken from a subject known or suspected of having a cancer (e.g., prostate cancer). The biological sample may be taken from a subject experiencing unexplained symptoms, such as fatigue, nausea, weight loss, aches and pains, weakness, or bleeding. The biological sample may be taken from a subject having explained symptoms. The biological sample may be taken from a subject at risk of developing cancer due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

After obtaining a biological sample from the subject, the biological sample may be processed to generate datasets indicative of a disease, condition, cancer-related category, or health state of the subject. For example, a tissue sample may be subjected to a histopathological assay (e.g., microscopy, including digital image acquisition such as whole slide imaging) to generate image data based on the biological sample. Alternatively, a liquid sample or a marker isolated from a sample may be subject to testing (e.g., a clinical laboratory test) to generate tabular data. In some embodiments, a sample is assayed for the presence, absence, or amount of one or more metabolites (e.g., prostate specific antigen (PSA)).

### Types of Data

Methods and systems as described herein make take as inputs one or more datasets. The one or more datasets may comprise tabular and/or image data. The tabular and/or image data may be derived from a biological sample of the subject. In some embodiments, the data are not derived form a biological sample.

The data may comprise images of tissue samples taken from a biopsy of a subject. The image data may be acquired by microscopy of the biopsy sample. The microscopy may comprise optical microscopy, virtual or digital microscopy (such as whole slide imaging (WSI)), or any suitable microscopy technique known in the field. The microscopy images may be subjected to one or more processing operations such as filtering, segmentation, concatenation, or object detection.

Tabular data as described herein may comprise any non-image data relevant to a health state or condition (e.g., disease) of a subject. Tabular data may comprise clinical data such as laboratory data at one or more timepoints (e.g., prostate serum antigen (PSA) level), qualitative measures of cell pathology (e.g., Gleason grade, Gleason score), structured or unstructured health data (e.g., digital rectal exam results), medical imaging data or results (e.g., results of an x-ray, computed tomography (CT) scan, magnetic resonance imaging (MRI) scan, positron-emission tomography (PET) scan, or ultrasound, such as transrectal ultrasound results), age, medical history, previous or current cancer state (e.g., remission, metastasis) or stage, current or previous therapeutic interventions, long-term outcome, and/or National Comprehensive Cancer Network (NCCN) classification or its constituents (e.g., combined Gleason score, t-stage, baseline PSA).

In some embodiments, the therapeutic intervention may comprise radiotherapy (RT). In some embodiments, the therapeutic intervention may comprise chemotherapy. In some embodiments, the therapeutic intervention may comprise a surgical intervention. In some embodiments, the therapeutic intervention may comprise an immunotherapy. In some embodiments, the therapeutic intervention may comprise a hormone therapy. In some embodiments, the therapeutic intervention may comprise an antiandrogen therapy. In some embodiments, the therapeutic intervention may comprise a nonsteroidal antiandrogen. In some embodiments, the therapeutic intervention may comprise one or more of Aminoglutethimide, Apalutamide, Bicalutamide, Enzalutamide, Flutamide, Ketoconazole, Nilutamide, or Topilutamide, In some embodiments, the RT may comprise RT with pre-specified use of short-term androgen deprivation therapy (ST-ADT). In some embodiments, the RT may comprise RT with pre-specified use of long-term ADT (LT-ADT). In some embodiments, the RT may comprise RT with pre-specified use of dose escalated RT (DE-RT). In some embodiments, the surgical intervention may comprise radical prostatectomy (RP). In some embodiments, the therapeutic intervention may comprise any combination of therapeutic interventions disclosed herein. In some embodiments, the long-term outcome may comprise distant metastasis (DM). In some embodiments, the long-term outcome may comprise biochemical recurrence (BR). In some embodiments, the long-term outcome may comprise partial response. In some embodiments, the long-term outcome may comprise complete response. In some embodiments, the long-term outcome may comprise death. In some embodiments, the long-term outcome may comprise relative survival. In some embodiments, the long-term outcome may comprise cancer-specific survival. In some embodiments, the cancer-specific survival may comprise prostate cancer-specific survival (PCaSS). In some embodiments, the long-term outcome may comprise progression free survival. In some embodiments, the long-term outcome may comprise disease free survival. In some embodiments, the long-term outcome may comprise metastasis-free survival (MFS). In some embodiments, the long-term outcome may comprise five-year survival. In some embodiments, the long-term outcome may comprise overall survival (OS). In some embodiments, the long-term outcome may comprise second progression-free survival (PFS2). In some embodiments, the long-term outcome may comprise any combination of long-term outcomes disclosed herein.

Data as used in methods and systems described herein may be subject to one or more processing operations. In some embodiments, data (e.g., image data) is subjected to an image processing, image segmentation, and/or object detection process as encoded in an image processing, image segmenting, or object detection algorithm. The image processing procedure may filter, transform, scale, rotate, mirror, shear, combine, compress, segment, concatenate, extract features from, and/or smooth an image prior to downstream processing. In some embodiments, a plurality of images (e.g., histopathology slides) is combined to form an image quilt. The image quilt may be converted to a representation (e.g., a tensor) that is useful for downstream processing of image data. The image segmentation process may partition an image into one or more segments which contain a factor or region of interest. For example, an image segmentation algorithm may process digital histopathology slides to determine a region of tissue as opposed to a region of whitespace or an artifact. In some embodiments, the image segmentation algorithm may comprise a machine learning or artificial intelligence algorithm. In some embodiments, image segmentation may precede image processing. In some embodiments, image processing may precede image segmentation. The object detection process may comprise detecting the presence or absence of a target object (e.g., a cell or cell part, such as a nucleus). In some embodiments, object detection may proceed image processing and/or image segmentation. For example, images which are found by an image detection algorithm to contain one or more objects of interest may be concatenated in a subsequent image processing operation. Alternatively, or additionally, image processing may precede object detection and/or image segmentation. For example, raw image data may be processed (e.g., filtered) and the processed image data subjected to an object detection algorithm. Image data may be subject to multiple image processing, image segmentation, and/or object detection operations in any appropriate order. In an example, image data is optionally subjected to one or more image processing operations to improve image quality. The processed image is then subjected to an image segmentation algorithm to detect regions of interest (e.g., regions of tissue in a set of histopathology slides). The regions of interest are then subjected to an object detection algorithm (e.g., an algorithm to detect nuclei in images of tissue) and regions found to possess at least one target object are concatenated to produce processed image data for downstream use.

In some embodiments, data (e.g., tabular data) may be subject to one or more processing operations. Processing operations may include, without limitation, standardization, or normalization. The one or more processing operations may, for example, discard data which contain spurious values or contain very few observations. The one or more processing operations may further or alternatively standardize the encoding of data values. Different input datasets may have the same parameter value encoded in different ways, depending on the source of the dataset. For example, ‘900’, ‘900.0’, ‘904’, ‘904.0’, ‘−1’, ‘−1.0’, ‘None’, and ‘NaN’ may all encode for a “missing” parameter value. The one or more processing operations may recognize the encoding variation for the same value and standardize the dataset to have a uniform encoding for a given parameter value. The processing operation may thus reduce irregularities in the input data for downstream use. The one or more data sets may normalize parameter values. For example, numerical data may be scaled, whitened, colored, decorrelated, or standardized. For example, data may be scaled or shifted to lie in a particular interval (e.g., [0,1] or [−1, 1]) and/or have correlations removed. In some embodiments, categorical data may be encoded as a one-hot vector. In some embodiments, one or more different types of tabular (e.g., numerical, categorical) data may be concatenated together. In some embodiments, data is not subjected to a processing operation.

Data may be taken at one or more timepoints. In some embodiments, data is taken at an initial timepoint and a later timepoint. The initial timepoint and the later timepoint may be spaced by any appropriate amount of time, such as 1 hour, 1 day, 1 week, 2 weeks, 3 weeks, 4 weeks, 6 weeks, 12 weeks, 4 months, 5 months, 6 months, 7 months, 8 months, 9 months, 10 months, 11 months, 1 years, 2 years, 3 years, 4 years, 5 years, 6 years, 7 years, 8 years, 9 years, 10 years, or more. In some embodiments, the data is from more than two timepoints. In some embodiments, the data are from 3, 4, 5, 6, 7, 8, 9, 10, 11, 12, 13, 14, 15, 16, 17, 18, 19, 20, or more timepoints. In some embodiments, the data are taken at a timepoint before a therapeutic intervention. In some embodiments, the data are taken at a timepoint subsequent to a therapeutic intervention. In some embodiments, data is taken at a timepoint before and after a therapeutic invention.

### Trained Algorithms

After using one or more assays to process one or more biological samples derived from the subject to generate one or more datasets indicative of the cancer state (e.g., cancer-related category or categories) of the subject, a trained algorithm may be used to process one or more of the datasets (e.g., a visual data and/or tabular data) to determine a cancer state of the subject. For example, the trained algorithm may be used to determine the presence or absence of (e.g., prostate) cancer in the subject based on the image data and/or laboratory data. The trained algorithm may be configured to identify the cancer state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise an unsupervised machine learning algorithm. The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm.

In some embodiments, a machine learning algorithm of a method or system as described herein utilizes one or more neural networks. In some case, a neural network is a type of computational system that can learn the relationships between an input dataset and a target dataset. A neural network may be a software representation of a human neural system (e.g., cognitive system), intended to capture “learning” and “generalization” abilities as used by a human. In some embodiments, the machine learning algorithm comprises a neural network comprising a CNN. Non-limiting examples of structural components of machine learning algorithms described herein include: CNNs, recurrent neural networks, dilated CNNs, fully-connected neural networks, deep generative models, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data in order to generate an output associated with that input.

In some embodiments, the neural network comprises artificial neural networks (ANNs). ANNs may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a deep neural network (DNN)) is an ANN comprising a plurality of hidden layers, e.g., two or more hidden layers. Each layer of the neural network may comprise a number of nodes (or “neurons”). A node receives input that comes either directly from the input data or the output of nodes in previous layers, and performs a specific operation, e.g., a summation operation. A connection from an input to a node is associated with a weight (or weighting factor). The node may sum up the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN computes are consistent with the examples included in the training dataset.

The number of nodes used in the input layer of the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater. In other instances, the number of nodes used in the input layer may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less. In some instances, the total number of layers used in the ANN or DNN (including input and output layers) may be at least about 3, 4, 5, 10, 15, 20, or greater. In other instances, the total number of layers may be at most about 20, 15, 10, 5, 4, 3, or less.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, or greater. In other instances, the number of learnable parameters may be at most about 100,000, 90,000, 80,000, 70,000, 60,000, 50,000, 40,000, 30,000, 20,000, 10,000, 9000, 8000, 7000, 6000, 5000, 4000, 3000, 2000, 1000, 900, 800, 700, 600, 500, 400, 300, 200, 100, 50, 10, or less.

In some embodiments of a machine learning algorithm as described herein, a machine learning algorithm comprises a neural network such as a deep CNN. In some embodiments in which a CNN is used, the network is constructed with any number of convolutional layers, dilated layers or fully-connected layers. In some embodiments, the number of convolutional layers is between 1-10 and the dilated layers between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or less, and the total number of dilated layers may be at most about 20, 15, 10, 5, 4, 3, or less. In some embodiments, the number of convolutional layers is between 1-10 and the fully-connected layers between 0-10. The total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. The total number of convolutional layers may be at most about 20, 15, 10, 5, 4, 3, 2, 1, or less, and the total number of fully-connected layers may be at most about 20, 15, 10, 5, 4, 3, 2, 1, or less.

In some embodiments, a machine learning algorithm comprises a neural network comprising a CNN, RNN, dilated CNN, fully-connected neural networks, deep generative models and/or deep restricted Boltzmann machines.

In some embodiments, a machine learning algorithm comprises one or more CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully-connected layers and normalization layers. The layers may be organized in 3 dimensions: width, height, and depth.

The convolutional layers may apply a convolution operation to the input and pass results of the convolution operation to the next layer. For processing images, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the width and height of the input volume, compute the dot product between the entries of the filter and the input, and produce a two-dimensional activation map of that filter. As a result, the network may learn filters that activate when it detects some specific type of feature at some spatial position in the input.

In some embodiments, a machine learning algorithm comprises an RNN. RNNs are neural networks with cyclical connections that can encode and process sequential data. An RNN can include an input layer that is configured to receive a sequence of inputs. An RNN may additionally include one or more hidden recurrent layers that maintain a state. At each operation, each hidden recurrent layer can compute an output and a next state for the layer. The next sate may depend on the previous state and the current input. The state may be maintained across operations and may capture dependencies in the input sequence.

An RNN can be a long short-term memory (LSTM) network. An LSTM network may be made of LSTM units. An LSTM unit may include of a cell, an input gate, an output gate, and a forget gate. The cell may be responsible for keeping track of the dependencies between the elements in the input sequence. The input gate can control the extent to which a new value flows into the cell, the forget gate can control the extent to which a value remains in the cell, and the output gate can control the extent to which the value in the cell is used to compute the output activation of the LSTM unit.

Alternatively, an attention mechanism (e.g., a transformer). Attention mechanisms may focus on, or “attend to,” certain input regions while ignoring others. This may increase model performance because certain input regions may be less relevant. At each operation, an attention unit can compute a dot product of a context vector and the input at the operation, among other operations. The output of the attention unit may define where the most relevant information in the input sequence is located.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully-connected layers connect every neuron in one layer to every neuron in another layer. In neural networks, each neuron may receive input from some number locations in the previous layer. In a fully-connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

The trained algorithm may be configured to accept a plurality of input variables and to produce one or more output values based on the plurality of input variables. The plurality of input variables may comprise one or more datasets indicative of a cancer-related category. For example, an input variable may comprise a microscopy image of a biopsy sample of the subject. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the biological sample and/or the subject by the classifier. The trained algorithm may comprise a binary classifier, such that each of the one or more output values comprises one of two values (e.g., {0, 1}, {positive, negative}, or {high-risk, low-risk}) indicating a classification of the biological sample and/or subject by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the biological sample and/or subject by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, high-risk, intermediate-risk, low-risk, or indeterminate. Such descriptive labels may provide an identification of a treatment for the subject's cancer-related category, and may comprise, for example, a therapeutic intervention, a duration of the therapeutic intervention, and/or a dosage of the therapeutic intervention suitable to treat a subject classified in a particular cancer-related category.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the cancer-related category of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a cancer-related state (e.g., type or stage of cancer) or belonging to a cancer-related category. For example, a binary classification of samples may assign an output value of “negative” or 0 if the sample indicates that the subject has less than a 50% probability of belonging to a cancer-related category. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject belongs to a cancer-related category (e.g., cancer diagnosis or prognosis) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of belonging to cancer-related category (e.g., long-term outcome) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a cancer-related state or belonging to a cancer-related category (e.g., positive for prostate cancer) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a cancer-related state (e.g., for prostate cancer) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a biological sample from a subject, associated datasets obtained by assaying the biological sample (as described elsewhere herein), clinical data form the subject, and one or more known output values corresponding to the biological sample and/or subject (e.g., a clinical diagnosis, prognosis, absence, or treatment efficacy of a cancer-related state of the subject). Independent training samples may comprise biological samples and associated datasets and outputs obtained or derived from a plurality of different subjects. Independent training samples may comprise biological samples and associated datasets and outputs obtained at a plurality of different time points from the same subject (e.g., on a regular basis such as weekly, biweekly, monthly, annually, etc.). Independent training samples may be associated with presence of the cancer-related state (e.g., training samples comprising biological samples and associated datasets and outputs obtained or derived from a plurality of subjects known to have the cancer-related state). Independent training samples may be associated with absence of the cancer-related state (e.g., training samples comprising biological samples and associated datasets and outputs obtained or derived from a plurality of subjects who are known to not have a previous diagnosis of the cancer-related state or who have received a negative test result for the cancer-related state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise cell-free biological samples and clinical data associated with presence of the cancer-related category and/or cell-free biological samples and clinical data associated with absence of the cancer-related category. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the cancer-related category. In some embodiments, the biological sample and/or clinical data is independent of samples used to train the trained algorithm.

The trained algorithm may be trained with a first number of independent training samples associated with presence of the cancer-related category and a second number of independent training samples associated with absence of the cancer-related category. The first number of independent training samples associated with presence of the cancer-related category may be no more than the second number of independent training samples associated with absence of the cancer-related category. The first number of independent training samples associated with presence of the cancer-related category may be equal to the second number of independent training samples associated with absence of the cancer-related category. The first number of independent training samples associated with presence of the cancer-related category may be greater than the second number of independent training samples associated with absence of the cancer-related category.

The trained algorithm may be configured to identify the cancer-related category at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the cancer-related category by the trained algorithm may be calculated as the percentage of independent test samples (e.g., subjects known to belong to the cancer-related category or subjects with negative clinical test results for the cancer-related category) that are correctly identified or classified as belonging to or not belonging to the cancer-related category.

The trained algorithm may be configured to identify the cancer-related category with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the cancer-related category using the trained algorithm may be calculated as the percentage of cell-free biological samples identified or classified as having the cancer-related category that correspond to subjects that truly belong to the cancer-related category.

The trained algorithm may be configured to identify the cancer-related category with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the cancer-related category using the trained algorithm may be calculated as the percentage of subject datasets identified or classified as not having the cancer-related category that correspond to subjects that truly do not belong to the cancer-related category.

The trained algorithm may be configured to identify the cancer-related category with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the cancer-related category using the trained algorithm may be calculated as the percentage of independent test samples associated with the cancer-related category (e.g., subjects known to belong to the cancer-related category) that are correctly identified or classified as having the cancer-related category.

The trained algorithm may be configured to identify the cancer-related category with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the cancer-related category using the trained algorithm may be calculated as the percentage of independent test samples associated with absence of the cancer-related category (e.g., subjects with negative clinical test results for the cancer-related category) that are correctly identified or classified as not belonging to the cancer-related category.

The trained algorithm may be configured to identify the cancer-related category with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying datasets derived from a subject as belonging to or not belonging to the cancer-related category.

The trained algorithm may be adjusted or tuned to improve one or more of the performance, accuracy, PPV, NPV, clinical sensitivity, clinical specificity, or AUC of identifying the cancer-related category. The trained algorithm may be adjusted or tuned by adjusting parameters of the trained algorithm (e.g., a set of cutoff values used to classify a biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the clinical data may be identified as most influential or most important to be included for making high-quality classifications or identifications of cancer-related categories (or sub-types of cancer-related categories). The clinical data or a subset thereof may be ranked based on classification metrics indicative of each parameter's influence or importance toward making high-quality classifications or identifications of cancer-related categories (or sub-types of cancer-related categories). Such metrics may be used to reduce, in some embodiments significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

Systems and methods as described herein may use more than trained algorithm to determine an output (e.g., cancer-related category of a subject). Systems and methods may comprise 1, 2, 3, 4, 5, 6, 7, 8, 9, 10, 11, 12, 13, 14, 15, 16, 17, 18, 19, 20, or more trained algorithms. A trained algorithm of the plurality of trained algorithms may be trained on a particular type of data (e.g., image data or tabular data). Alternatively, a trained algorithm may be trained on more than one type of data. The inputs of one trained algorithm may comprise the outputs of one or more other trained algorithms. Additionally, a trained algorithm may receive as its input the output of one or more trained algorithms.

### Identifying or Monitoring a Cancer-Related Category or State

After using a trained algorithm to process the dataset, the cancer-related category or may be identified or monitored in the subject. The identification may be based at least in part on quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites.

The cancer-related category may characterize a cancer-related state of the subject. By way of nonlimiting example, the cancer related state may comprise a subject having or not having a cancer (e.g., prostate cancer), a subject being at risk or having a risk level (e.g., high risk, low risk) for a cancer, a predicted long-term outcome of a cancer (e.g., distant metastasis, biochemical recurrence, partial response, complete response, overall survival, cancer-specific survival, progression free survival, second progression-free survival, metastasis-free survival, disease free survival, five-year survival, death), response or receptiveness to a therapeutic intervention, or any combination thereof.

The subject may be identified as belonging to a cancer-related category at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the cancer-related category of the individual by the trained algorithm may be calculated as the percentage of independent test samples (e.g., subjects known to belong to the cancer-related category or subjects with negative clinical test results corresponding to the cancer-related category) that are correctly identified or classified as belonging to or not belonging to the cancer-related category.

The subject may be determined as belonging to a cancer-related category with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the cancer-related category using the trained algorithm may be calculated as the percentage of biological samples identified or classified as belonging to the cancer-related category that correspond to subjects that truly belong to the cancer-related category.

The cancer-related category may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the cancer-related category using the trained algorithm may be calculated as the percentage of biological samples identified or classified as not having the cancer-related category that correspond to subjects that truly do not have the cancer-related category.

The subject may be identified as belonging to the cancer-related category with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the cancer-related category using the trained algorithm may be calculated as the percentage of independent test samples associated with belonging to the cancer-related category (e.g., subjects known to belong to the cancer-related category) that are correctly identified or classified as belonging to the cancer-related category.

The cancer-related category may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the cancer-related category using the trained algorithm may be calculated as the percentage of independent test samples associated with not belonging to the cancer-related category (e.g., subjects with negative clinical test results for the cancer-related category) that are correctly identified or classified as not belonging to the cancer-related category.

After the cancer-related category is identified in a subject, a sub-type of the cancer-related category (e.g., selected from among a plurality of sub-types of the cancer-related category) may further be identified. The sub-type of the cancer-related category may be determined based at least in part on quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites. For example, the subject may be identified as being at risk of a sub-type of prostate cancer (e.g., from among a number of sub-types of prostate cancer). After identifying the subject as being at risk of a sub-type of prostate cancer, a clinical intervention for the subject may be selected based at least in part on the sub-type of prostate cancer for which the subject is identified as being at risk. In some embodiments, the clinical intervention is selected from a plurality of clinical interventions (e.g., clinically indicated for different sub-types of prostate cancer, such as nonmetastatic castration-resistant prostate cancer).

Upon identifying the subject as belonging to the cancer-related category, the subject may be optionally provided with a therapeutic intervention (e.g., prescribing an appropriate course of treatment to treat the type, sub-type, or state of the cancer of the subject). The therapeutic intervention may comprise a prescription of an effective dose of a drug (e.g., a nonsteroidal antiandrogen) or other therapy (e.g., radiotherapy, chemotherapy), a surgical intervention (e.g., radical prostatectomy), a further testing or evaluation of the cancer-related category, a further monitoring of the cancer-related category, or a combination thereof. If the subject is currently being treated for the cancer-related category with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

The analysis of biopsy samples (e.g., analysis of microscopy images of prostate tissue), proteomic data comprising quantitative measures of proteins of the dataset at a panel of cancer-related category-associated proteins, and/or metabolome data comprising quantitative measures of a panel of cancer-related category-associated metabolites may be assessed over a duration of time to monitor a patient (e.g., subject who has or is at risk for cancer or who is being treated for a cancer). In such cases, the measures of the dataset of the patient may change during the course of treatment. For example, the measures of the dataset of a patient with decreasing risk of the cancer-related category due to an effective treatment may shift toward the profile or distribution of a healthy subject (e.g., a subject without a cancer or in remission from cancer). Conversely, for example, the measures of the dataset of a patient with increasing risk of the cancer-related category due to an ineffective treatment may shift toward the profile or distribution of a subject with higher risk of the cancer-related category or a more advanced cancer-related category.

The cancer-related category of the subject may be monitored by monitoring a course of treatment for treating the cancer or cancer-related state of the subject. The monitoring may comprise assessing the cancer-related category or state of the subject at two or more time points. The assessing may be based at least on quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined at each of the two or more time points.

In some embodiments, a difference in quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of one or more clinical indications, such as (i) a diagnosis of the cancer-related state of the subject, (ii) a prognosis of the cancer-related state of the subject, (iii) an increased risk of the cancer-related state of the subject, (iv) a decreased risk of the cancer-related state of the subject, (v) an efficacy of the course of treatment for treating the cancer-related state of the subject, and (vi) a non-efficacy of the course of treatment for treating the cancer-related state of the subject.

In some embodiments, a difference in quantitative or qualitative measures of biological samples (e.g., of histopathology slides of biopsy samples), proteomic data comprising quantitative measures of proteins of the dataset at a panel of cancer-related category-associated proteins, and/or metabolome data comprising quantitative measures of a panel of cancer-associated metabolites determined between the two or more time points may be indicative of a diagnosis of the cancer-related state or category of the subject. For example, if the cancer-related state was not detected in the subject at an earlier time point but was detected in the subject at a later time point, then the difference is indicative of a diagnosis of the cancer-related state of the subject. A clinical action or decision may be made based on this indication of diagnosis of the cancer-related state of the subject, such as, for example, prescribing a new therapeutic intervention for the subject. The clinical action or decision may comprise recommending the subject for a secondary clinical test to confirm the diagnosis of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of a prognosis of the cancer-related category of the subject.

In some embodiments, a difference in the quantitative or qualitative measures of biological samples (e.g., of histopathology slides of biopsy samples), proteomic data comprising quantitative measures of proteins of the dataset at a panel of cancer-related category-associated proteins, and/or metabolome data comprising quantitative measures of a panel of cancer-associated metabolites determined between the two or more time points may be indicative of the subject having an increased risk of the cancer-related state. For example, if the cancer-related state was detected in the subject both at an earlier time point and at a later time point, and if the difference is a negative difference (e.g., the quantitative or qualitative measures of biological samples (e.g., of histopathology slides of biopsy samples), proteomic data comprising quantitative measures of proteins of the dataset at a panel of cancer-related category-associated proteins, and/or metabolome data comprising quantitative measures of a panel of cancer-related category-associated metabolites increased from the earlier time point to the later time point), then the difference may be indicative of the subject having an increased risk of the cancer-related state. A clinical action or decision may be made based on this indication of the increased risk of the cancer-related state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of the subject having a decreased risk of the cancer-related state. For example, if the cancer-related state was detected in the subject both at an earlier time point and at a later time point, and if the difference is a positive difference (e.g., the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites decreased from the earlier time point to the later time point), then the difference may be indicative of the subject having a decreased risk of the cancer-related state. A clinical action or decision may be made based on this indication of the decreased risk of the cancer-related state (e.g., continuing or ending a current therapeutic intervention) for the subject. The clinical action or decision may comprise recommending the subject for a secondary clinical test to confirm the decreased risk of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of an efficacy of the course of treatment for treating the cancer-related state of the subject. For example, if the cancer-related state was detected in the subject at an earlier time point but was not detected in the subject at a later time point, then the difference may be indicative of an efficacy of the course of treatment for treating the cancer-related state of the subject. A clinical action or decision may be made based on this indication of the efficacy of the course of treatment for treating the cancer-related state of the subject, e.g., continuing or ending a current therapeutic intervention for the subject. The clinical action or decision may comprise recommending the subject for a secondary clinical test to confirm the efficacy of the course of treatment for treating the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

In some embodiments, a difference in the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites determined between the two or more time points may be indicative of a non-efficacy of the course of treatment for treating the cancer-related category of the subject. For example, if the cancer-related state was detected in the subject both at an earlier time point and at a later time point, and if the difference is a negative or zero difference (e.g., the quantitative or qualitative measures of biological samples (e.g., of histopathology slides of biopsy samples), proteomic data comprising quantitative measures of proteins of the dataset at a panel of cancer-associated proteins, and/or metabolome data comprising quantitative measures of a panel of cancer-associated metabolites increased or remained at a constant level from the earlier time point to the later time point), and if an efficacious treatment was indicated at an earlier time point, then the difference may be indicative of a non-efficacy of the course of treatment for treating the cancer-related state of the subject. A clinical action or decision may be made based on this indication of the non-efficacy of the course of treatment for treating the cancer-related state of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the cancer-related state. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

### Outputting a Report of the Cancer-Related State

After the cancer-related state is identified or an increased risk of the cancer-related state is monitored in the subject, a report may be electronically outputted that is indicative of (e.g., identifies or provides an indication of) the cancer-related state of the subject. The subject may not display a cancer-related state (e.g., is asymptomatic of the cancer-related state such as a presence or risk of prostate cancer). The report may be presented on a graphical user interface (GUI) of an electronic device of a user. The user may be the subject, a caretaker, a physician, a nurse, or another health care worker.

The report may include one or more clinical indications such as (i) a diagnosis of the cancer-related state of the subject, (ii) a prognosis of the cancer-related category of the subject, (iii) an increased risk of the cancer-related category of the subject, (iv) a decreased risk of the cancer-related category of the subject, (v) an efficacy of the course of treatment for treating the cancer-related category of the subject, (vi) a non-efficacy of the course of treatment for treating the cancer-related category of the subject, and (vii) a long-term outcome of the cancer-related category. The report may include one or more clinical actions or decisions made based on these one or more clinical indications. Such clinical actions or decisions may be directed to therapeutic interventions or further clinical assessment or testing of the cancer-related state of the subject.

For example, a clinical indication of a diagnosis of the cancer-related state of the subject may be accompanied with a clinical action of prescribing a new therapeutic intervention for the subject. As another example, a clinical indication of an increased risk of the cancer-related state of the subject may be accompanied with a clinical action of prescribing a new therapeutic intervention or switching therapeutic interventions (e.g., ending a current treatment and prescribing a new treatment) for the subject. As another example, a clinical indication of a decreased risk of the cancer-related state of the subject may be accompanied with a clinical action of continuing or ending a current therapeutic intervention for the subject. As another example, a clinical indication of an efficacy of the course of treatment for treating the cancer-related state of the subject may be accompanied with a clinical action of continuing or ending a current therapeutic intervention for the subject. As another example, a clinical indication of a non-efficacy of the course of treatment for treating the cancer-related state of the subject may be accompanied with a clinical action of ending a current therapeutic intervention and/or switching to (e.g., prescribing) a different new therapeutic intervention for the subject.

In some embodiments, the therapeutic intervention may comprise radiotherapy (RT), chemotherapy, (anti)hormone therapy (e.g., antiandrogen therapy, such as a nonsteroidal antiandrogen therapy), a surgical intervention (e.g., radical prostatectomy), a further testing or evaluation of the cancer-related category, a further monitoring of the cancer-related category, or a combination thereof. If the subject is currently being treated for the cancer-related category with a course of treatment, the therapeutic intervention may comprise a subsequent different course of treatment (e.g., to increase treatment efficacy due to non-efficacy of the current course of treatment). The therapeutic intervention may comprise recommending the subject for a secondary clinical test to confirm a diagnosis of the cancer-related category. This secondary clinical test may comprise an imaging test, a blood test, a computed tomography (CT) scan, a magnetic resonance imaging (MRI) scan, an ultrasound scan, an X-ray, a positron emission tomography (PET) scan, a PET-CT scan, a bone scan, a lymph node biopsy, or any combination thereof.

### Computer Systems

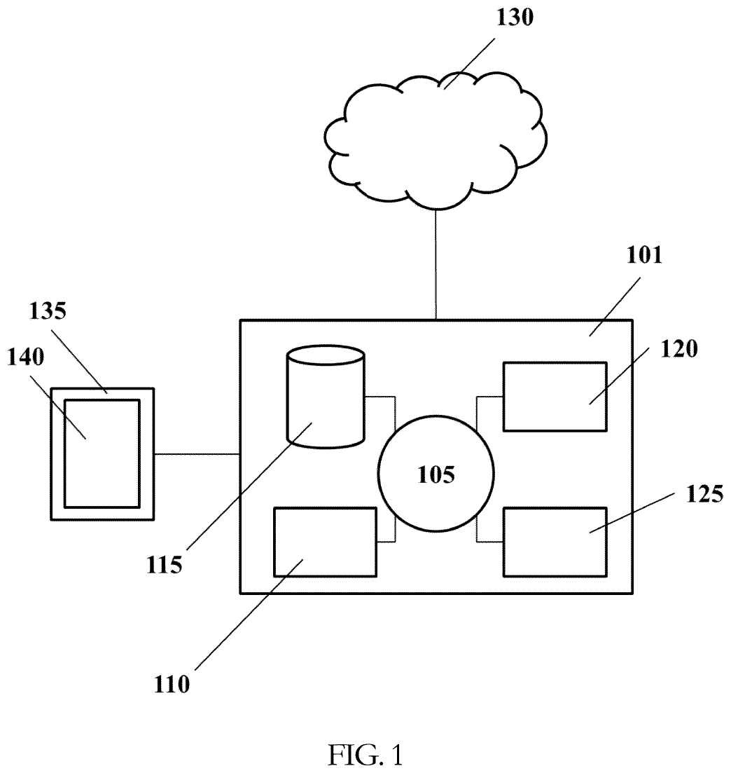

The present disclosure provides computer systems that are programmed to implement methods of the disclosure. FIG. 1 shows a computer system 101 that is programmed or otherwise configured to, for example, (i) train and test a trained algorithm, (ii) use the trained algorithm to process image and/or tabular data to determine a cancer-related category or cancer-related state of a subject, (iii) assess a cancer of the subject based on a classified category, (iv) identify or monitor the cancer-related category or state of the subject, and (v) electronically output a report that indicative of the cancer-related category or state of the subject.

The computer system 101 can regulate various aspects of analysis, calculation, and generation of the present disclosure, such as, for example, (i) training and testing a trained algorithm, (ii) using the trained algorithm to process image and/or tabular data to determine a cancer-related category or cancer-related state of a subject, (iii) assessing a cancer of the subject based on a classified category, (iv) identifying or monitoring the cancer-related category or state of the subject, and (v) electronically outputting a report that indicative of the cancer-related category or state of the subject. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet.

In some embodiments, the network 130 is a telecommunication and/or data network. The network 130 can include one or more computer servers, which can enable distributed computing, such as cloud computing. For example, one or more computer servers may enable cloud computing over the network 130 (“the cloud”) to perform various aspects of analysis, calculation, and generation of the present disclosure, such as, for example, (i) training and testing a trained algorithm, (ii) using the trained algorithm to process data to determine a cancer-related category of a subject, (iii) determining a quantitative measure indicative of a cancer-related category of a subject, (iv) identifying or monitoring the cancer-related category of the subject, and (v) electronically outputting a report that indicative of the cancer-related category of the subject. Such cloud computing may be provided by cloud computing platforms such as, for example, Amazon Web Services (AWS), Microsoft Azure, Google Cloud Platform, and IBM cloud. In some embodiments, the network 130, with the aid of the computer system 101, can implement a peer-to-peer network, which may enable devices coupled to the computer system 101 to behave as a client or a server.

The CPU 105 may comprise one or more computer processors and/or one or more graphics processing units (GPUs). The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 115 can store files, such as drivers, libraries, and saved programs. The storage unit 115 can store user data, e.g., user preferences and user programs. In some embodiments, the computer system 101 can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine-readable code can be provided in the form of software. During use, the code can be executed by the processor 105. In some embodiments, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Embodiments of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, (i) a visual display indicative of training and testing of a trained algorithm, (ii) a visual display of data indicative of a cancer-related category of a subject, (iii) a quantitative measure of a cancer-related category of a subject, (iv) an identification of a subject as having a cancer-related category, or (v) an electronic report indicative of the cancer-related category of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, (i) train and test a trained algorithm, (ii) use the trained algorithm to process image and/or tabular data to determine a cancer-related category or cancer-related state of a subject, (iii) assess a cancer of the subject based on a classified category, (iv) identify or monitor the cancer-related category or state of the subject, and (v) electronically output a report that indicative of the cancer-related category or state of the subject.

### EXAMPLES

### Example 1: Prostate Cancer Therapy Personalization Via Multi-Modal Deep Learning

Methods and systems as disclosed herein demonstrate prostate cancer therapy personalization by predicting long-term, clinically relevant outcomes (distant metastasis, biochemical recurrence, death from prostate cancer, and overall survival) using multimodal deep learning models trained on digital histopathology of prostate biopsies and clinical data. An example system of the present disclosure comprises a trained algorithm that was trained and validated using a dataset of five phase III randomized multinational trials run across hundreds of clinical sites. Clinical and histopathological data was available for 5,654 of 7,957 patients (71.1%), which yielded 16.1 terabytes of histopathology imagery, with 10-20 years of patient follow-up. Compared to the most commonly used risk stratification tool, the National Cancer Center Network's (NCCN) risk groups, the deep learning model had superior prognostic and discriminatory performance across all tested outcomes. This artificial intelligence system may allow oncologists to computationally model the likeliest outcomes of any specific patient and thus determine their optimal treatment. Outfitted with digital histopathology scanners and internet access, any clinic could offer such capabilities, enabling low-cost universal access to vital therapy personalization.

The NCCN risk groups are based on the international standards for risk stratification, developed in the late 1990s and referred to as the D'Amico risk groups. This system is based on a digital rectal exam, a serum prostate-specific antigen (PSA) measurement, and the grade of a tumor assessed by histopathology. This three-tier system continues to form the backbone of treatment recommendations throughout the world but has suboptimal prognostic and discriminatory performance to risk stratify patients. This in part is due to the highly subjective and non-specific nature of the core variables in these models. For instance, Gleason grading was developed in the 1960s and remains highly subjective, with unacceptable interobserver reproducibility even amongst expert urologic pathologists. More recently, tissue-based genomic biomarkers have demonstrated improved prognostic performance. However, nearly all of these tests lack validation in prospective randomized clinical trials in the intended use population, and there has been little to no international adoption due to costs and processing time. As such, there remains a serious unmet clinical need for improved tools to personalize therapy for prostate cancer.

Artificial intelligence (AI) has demonstrated remarkable capabilities across a number of use-cases in medicine, ranging from physician-level diagnostics to workflow optimization, and has the potential to support cancer therapy as clinical adoption of digital histopathology continues. AI has begun making progress in histopathology-based prognostics, for instance by predicting short-term patient outcomes or by improving the accuracy of Gleason-based cancer grading on postoperative surgical samples. Whereas standard risk-stratification tools are fixed and based on few variables, AI can learn from large amounts of minimally processed data across various modalities. In contrast to genomic biomarkers, AI systems are low-cost, massively scalable, and incrementally improve through usage. Furthermore, a key challenge for any biomarker is having optimal data to train and validate the relevant endpoint(s), and some commercial prognostic biomarkers in oncology may have been trained on retrospective convenience sampling.

Methods and systems as disclosed herein may comprise a multimodal artificial intelligence (MMAI) system that can meaningfully overcome the unmet need for outcomes prognostication in localized prostate cancer, creating a generalizable biomarker with the potential for global adoption. Prognostic biomarkers localized prostate cancer using five phase III randomized clinical trials were used to train an algorithm as described herein by leveraging multi-modal deep learning on digital histopathology.

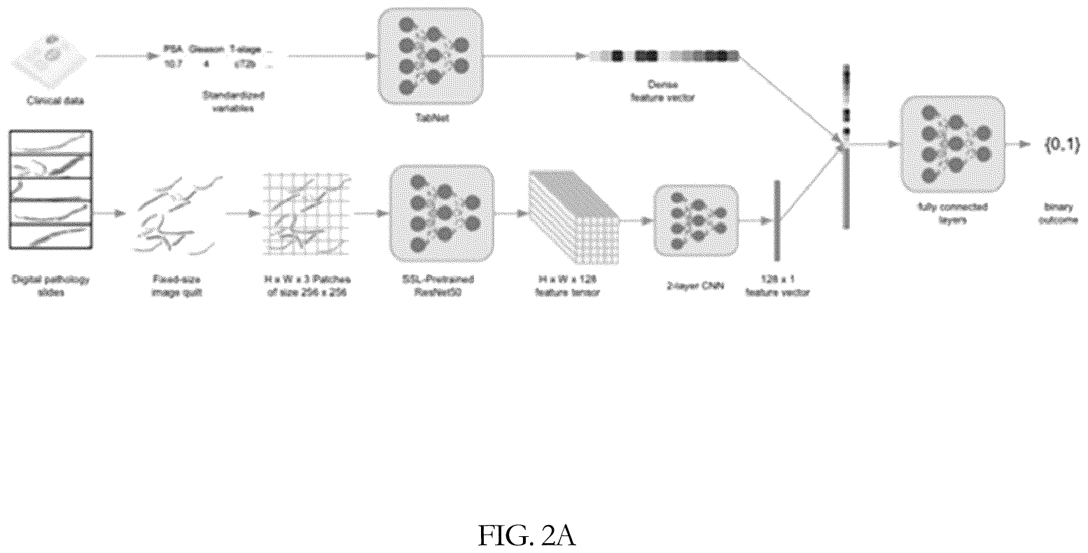

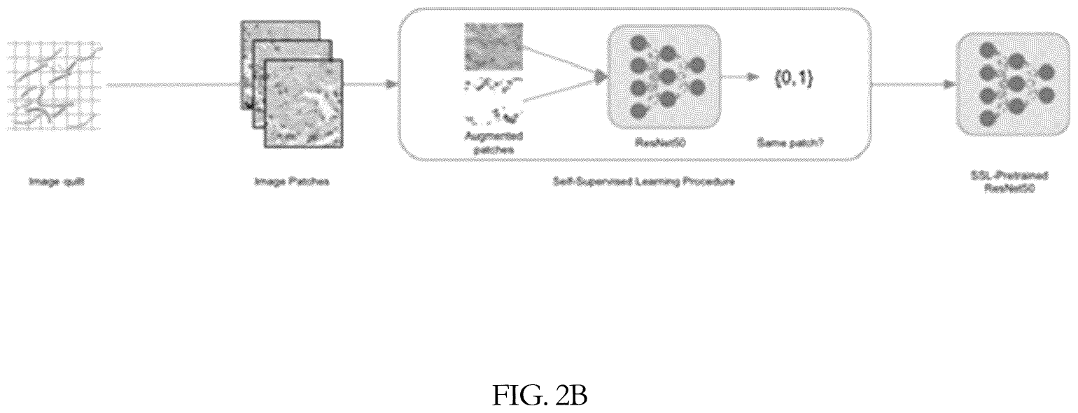

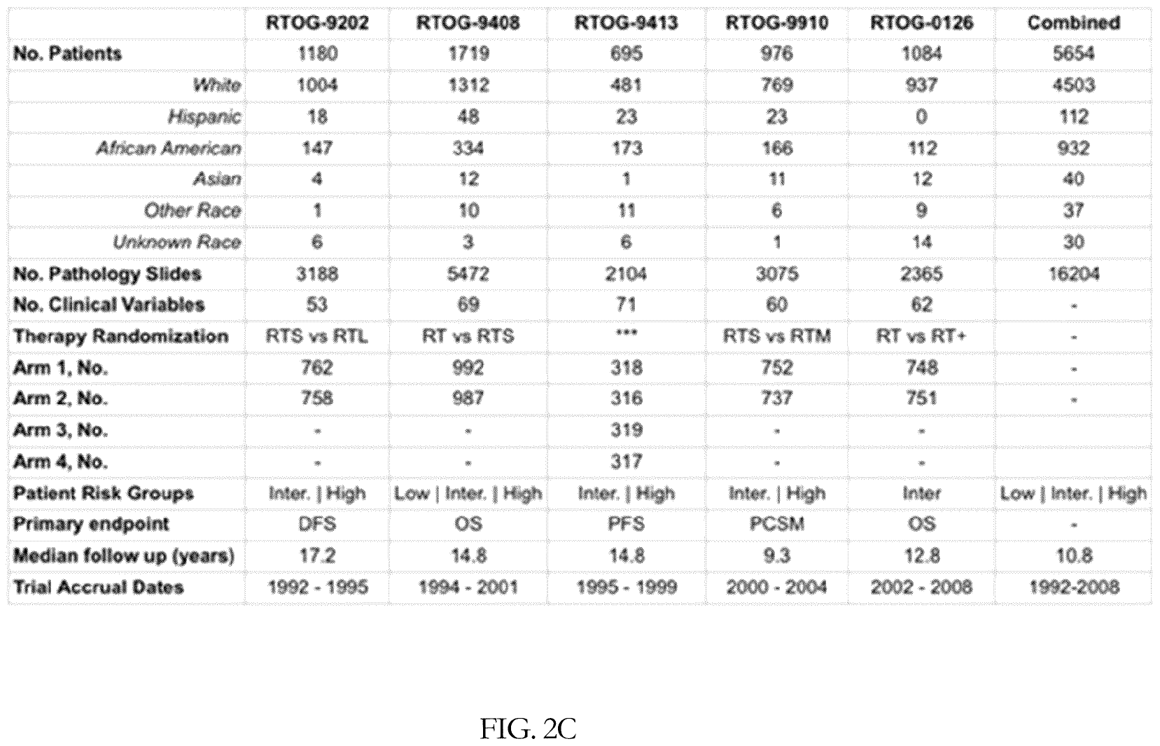

A unique dataset from five large multi-national randomized phase III clinical trials of men with localized prostate cancer (NRG/RTOG 9202, 9408, 9413, 9910, and 0126) was generated. All patients received definitive radiotherapy (RT), with pre-specified use of short-term androgen deprivation therapy (ST-ADT), long-term ADT (LT-ADT), and/or dose-escalated RT (DE-RT) (FIG. 2C). Of the 7,957 patients enrolled in these five trials, there were 7,752 patients with complete baseline clinical data and 5,654 with complete baseline and digital histopathology data. This represents 16.1 TB of histopathology imagery from 16,204 histopathology slides of pretreatment biopsy samples.

The MMAI architecture can ingest both tabular (clinical) and image-based (histopathology) data, making it uniquely suited for randomized clinical trial data. The full architecture is shown in FIG. 2A. Each patient in the dataset is represented by clinical variables—including laboratory and pathology data, therapeutic interventions, and long-term outcomes—and digitized histopathology slides (median of 3.5 slides). Joint learning across both data streams is complex and involves building three separate deep learning pipelines—one for the imagery, one for the tabular data, and a third to unite them. The data were standardized across the trials for consistency.

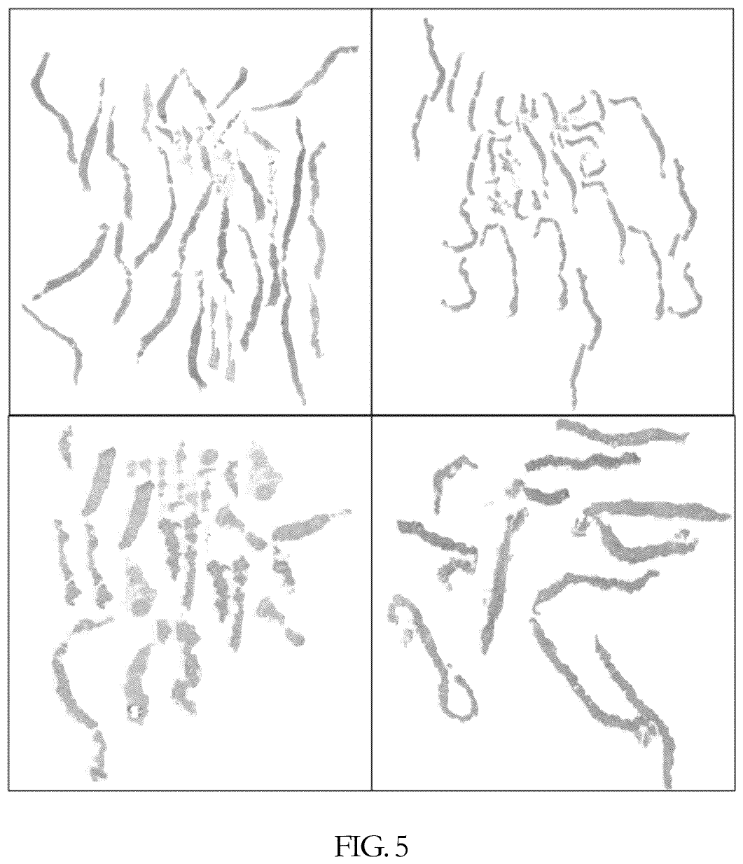

Effective learning of relevant features from a variable number of digitized histopathology slides involved several pre-processing operations to standardize the images, followed by self-supervised training. For each patient, all the tissue sections in their biopsy slides were segmented out and combined into a single large image, called an image quilt (FIG. 5), of a fixed width and height across all patients. An H×W grid was overlain on the image quilt which cut it into patches of size 256×256 pixels across its RGB channels. These patches were then used to train a self-supervised (SSL) model to learn histopathological features useful for downstream AI tasks. FIG. 2B shows this part of the pipeline. Once trained, the SSL model could then take the patches of an image quilt and output a 128-dimensional vector representation for each patch. Concatenating all of these vectors in the same spatial orientation as the original patches yielded an H×W×128 tensor (a feature-quilt) that compressed the initially massive image quilt into a compact representation useful for further downstream learning.

SSL is a method that may be used for learning from datasets without annotations. Typical ML setups leverage supervised learning, in which datasets are composed of data points (e.g., images) and data labels (e.g., object classes). In contrast, during SSL, synthetic data labels are extracted from the original data and used to train generic feature representations which can be used for downstream tasks. Momentum contrast—a technique which takes the set of image patches, generates augmented copies of each patch, then trains a model to predict whether any two augmented copies come from the same original patch—may be effective at learning features for medical tasks. The structural setup is shown in FIG. 2B, with further details described elsewhere herein.

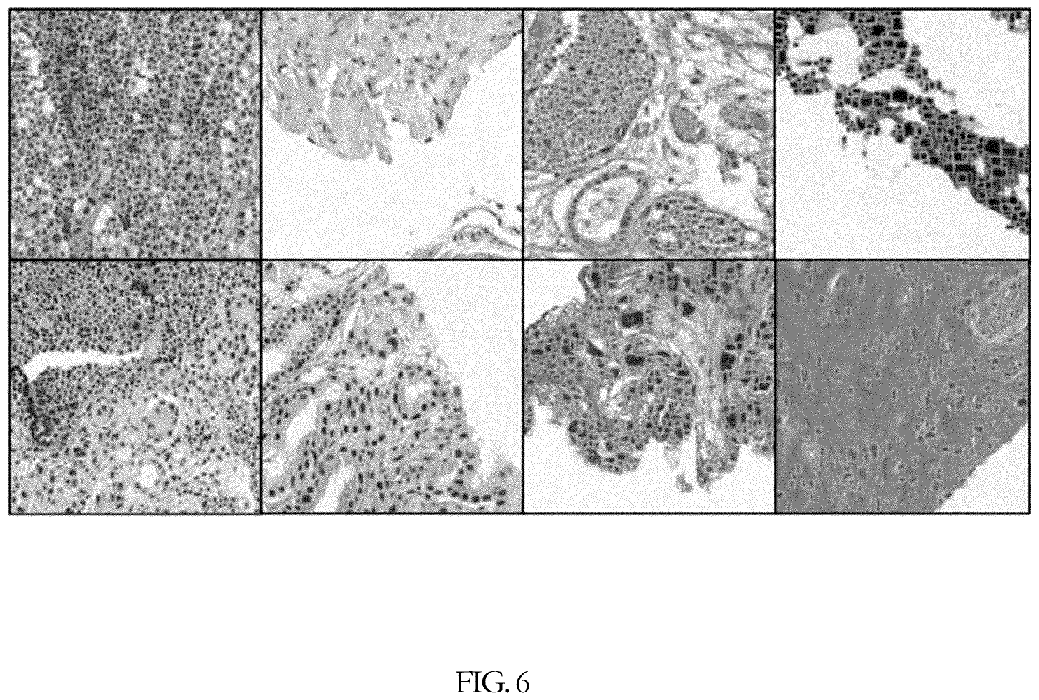

To guide the SSL process towards patch regions that are likely to be more clinically useful, patches in the dataset were oversampled based on nucleic density. Using an object detection model trained to detect nuclei the number of nuclei in each patch was approximated. The patches were binned into deciles based on this count, and each decile was oversampled such that the net number of images seen during one epoch of training is the same for each decile. Examples images are shown in FIG. 6.

Systems as described herein may learn from patient-level annotations with the histopathology slides left unannotated. Moreover, the self-supervised learning of the image model allows it to learn from new image data without the need for any annotations.

Learning from the tabular data comprised two operations. First, the clinical data was standardized across the trials and used to pre-train a TabNet architecture via self-supervision by masking parts of data and training the model to learn them. Then, each patient's data was fed through TabNet to extract a feature vector, which was then concatenated with the output of the image pipeline. The concatenated vector was then fed through further neural layers and the model output a binary outcome for the task at hand.

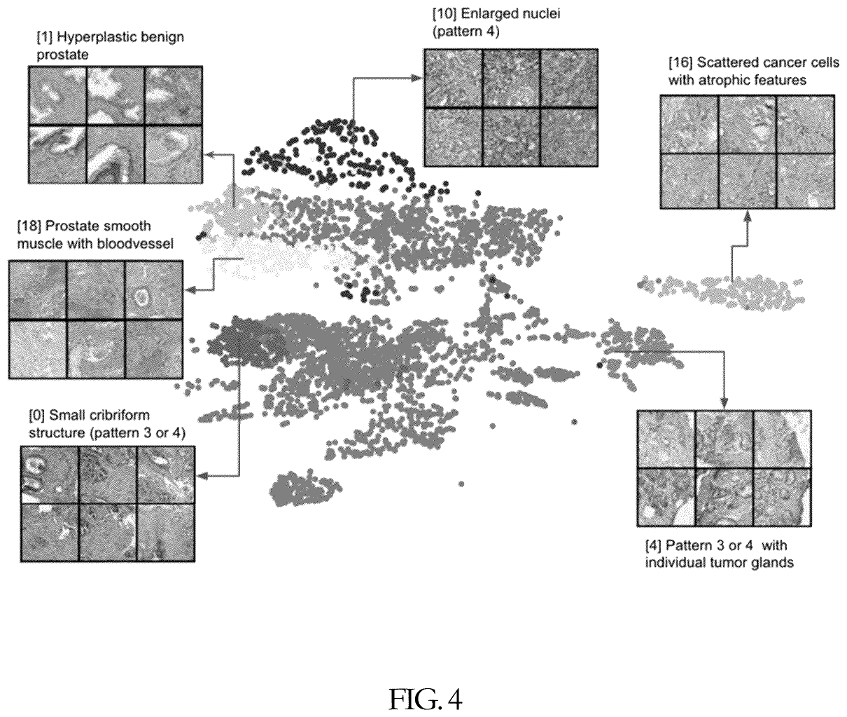

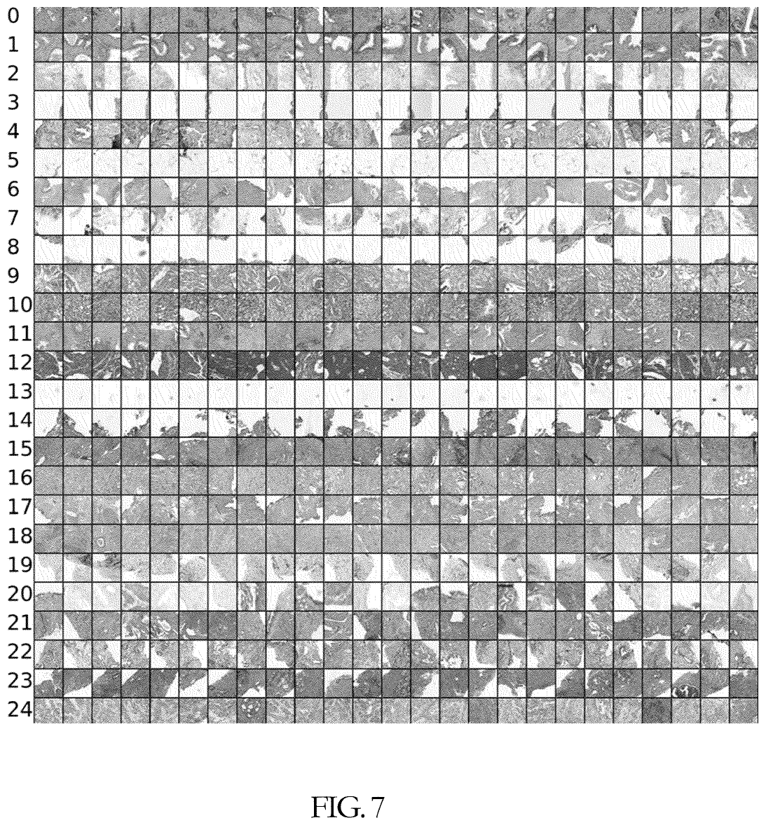

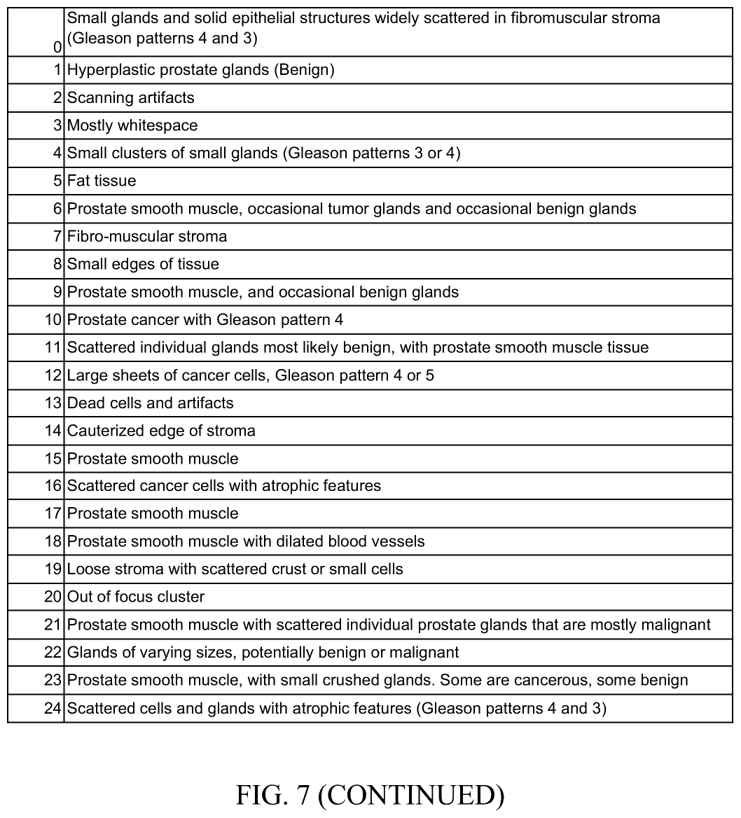

The internal data representations of the SSL model are shown in FIG. 4. The entire dataset's image patches were fed through the SSL model and model features-a 128-dimensional vector outputted by the model-were extracted for each patch. Uniform Manifold Approximation and Projection algorithm (UMAP) was then applied to these features, projecting them from 128 dimensions down to two, and each patch was plot as an individual point. Neighboring data points represent image patches that the model considered similar. UMAP grouped the feature vectors into 25 clusters, some of which are shown in various colors. Insets show example image patches that are close in feature space to cluster centroids. The 20 nearest-neighbor image patches to the cluster centroids were then interpreted by a pathologist. Example interpretations are shown in FIG. 4, and the full interpretation is shown in FIG. 7. The SSL model learned human-interpretable image features that are indicative of complex aspects of cancer, such as Gleason grade or tissue type, despite never being trained with clinical annotations.

Six different MMAI models were trained and tested across four endpoints (DM, BCR, PCaSS, OS) and two timeframes: 5-year and 10-year. The performance of these models was measured with the area under the time-dependent receiver operator characteristic curve (AUC) of sensitivity and specificity, accounting for competing events. Sensitivity is defined as the ratio of correct positive predictions to positive events shown (sensitivity=predicted_positive/num_positive), and the specificity is defined as the ratio of correct negative predictions to negative events shown (specificity=predicted_negative/num_negative). For this metric, 0.5 denotes random chance accuracy and 1.0 denotes perfect accuracy.

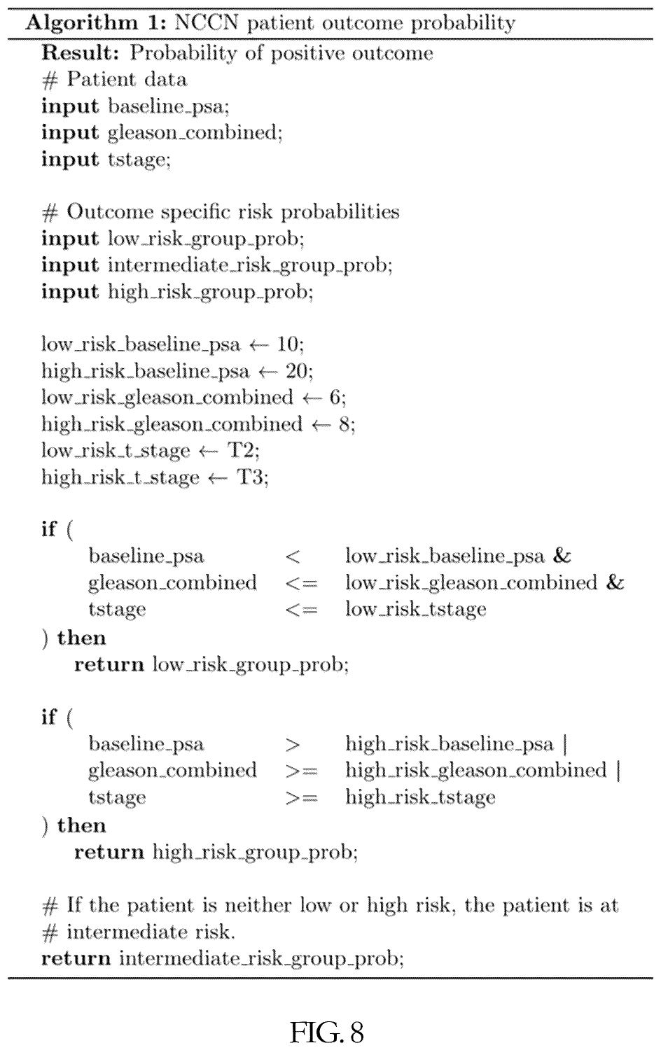

The NCCN model served as the baseline comparator, as shown in FIG. 8. Three variables—Gleason score, tumor t-stage, and baseline PSA—were used to group patients into low-, intermediate-, and high-risk groups.

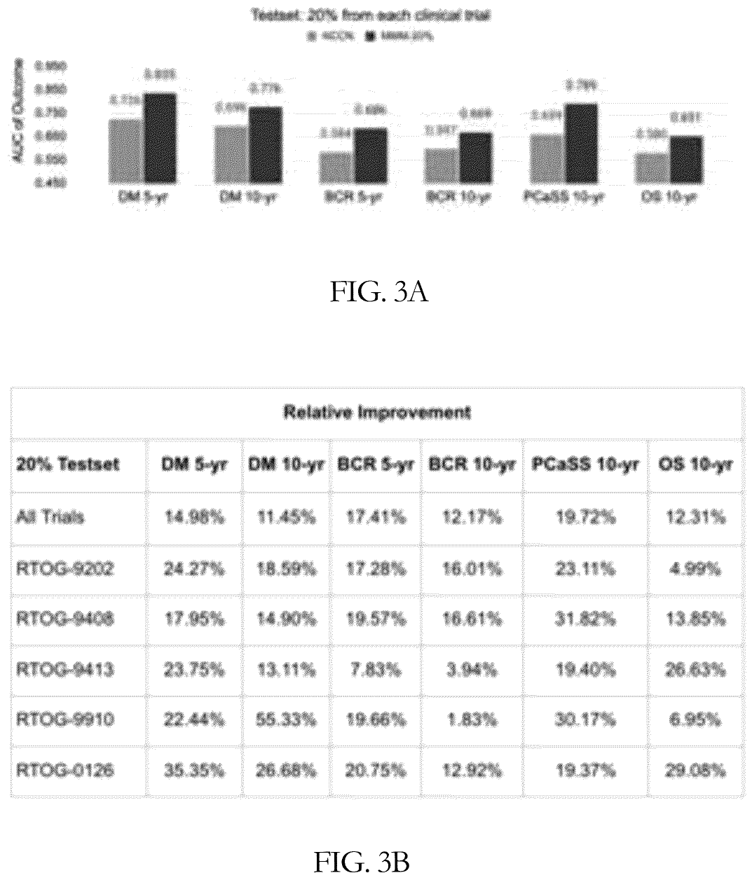

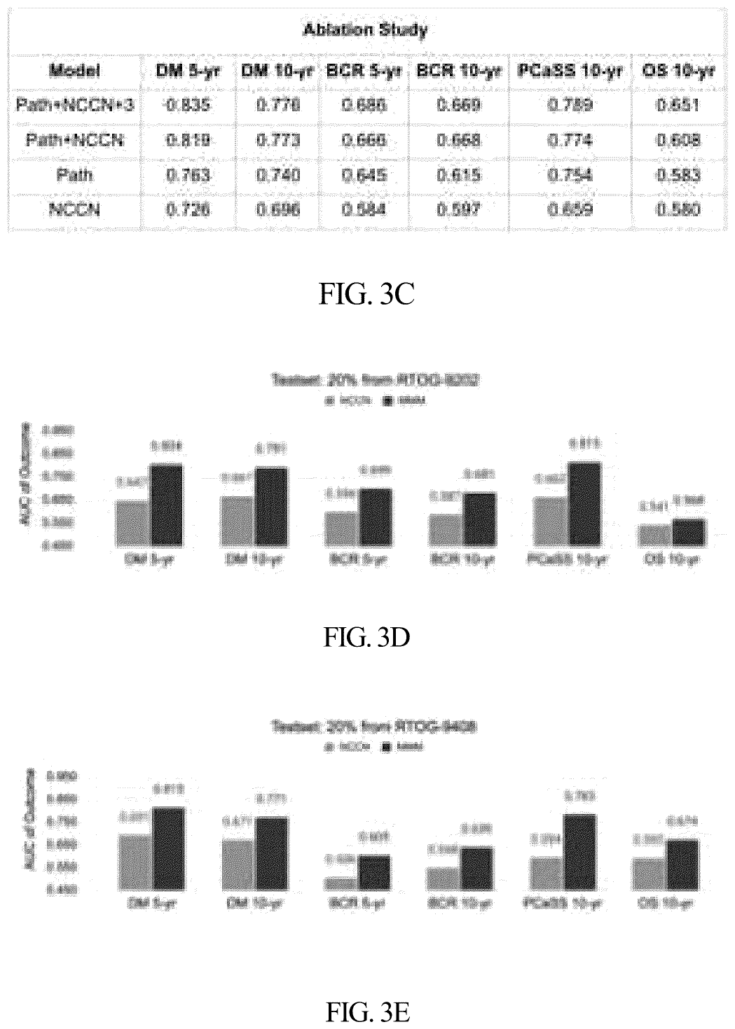

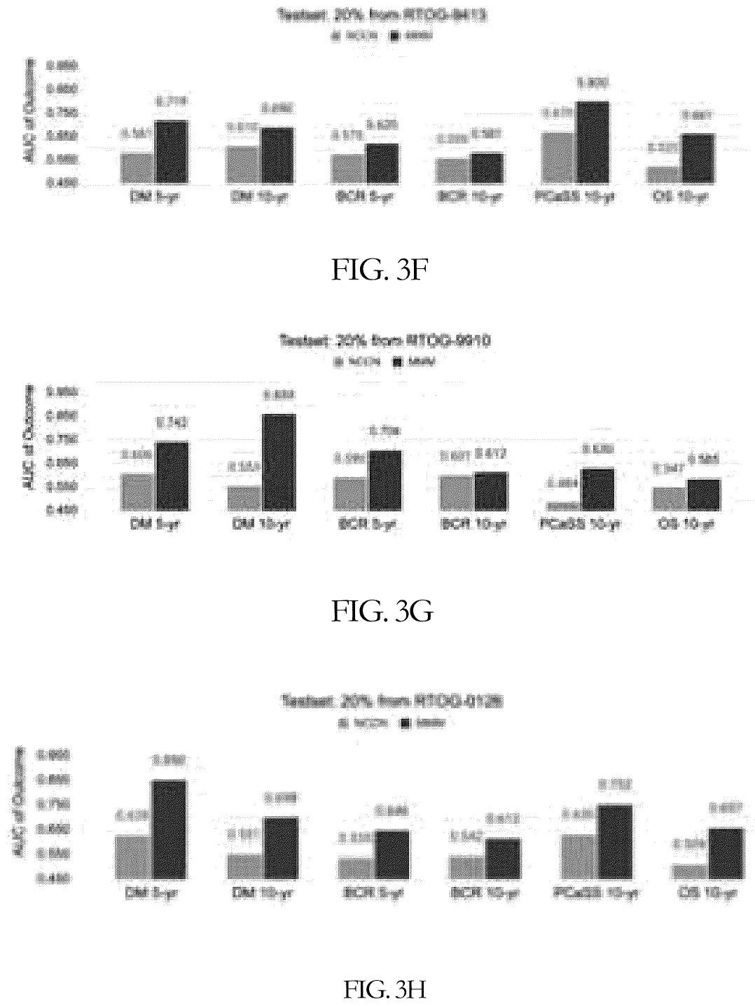

The results are shown in FIGS. 3A-3H. A separate model was trained for each outcome and time point. In FIG. 3A and FIG. 2D-2H, the blue bars represent the performance of an MMAI model trained on a specific task and the gray bars represent the performance of the corresponding NCCN model. FIG. 2B shows the relative improvement of the MMAI over NCCN across the outcomes and across the subsets of the test set that come from the five trials. The MMAI model consistently outperformed the NCCN model across all tested outcomes. The relative improvement in AUC varied from 11.45% up to 19.72%. Further, the trial subsets unanimously saw a relative improvement over NCCN.

To evaluate the effects of the various data components specific to the MMAI model, an ablation study was run. Additional MMAI models were trained using the following data setups: NCCN variables only, pathology images only, pathology images+the NCCN variables (combined Gleason score, t-stage, baseline PSA), and pathology images+NCCN variables+3 additional variables used in the model (age, Gleason primary, Gleason secondary). Each additional data component improved performance, with the full setup (pathology, 6 clinical variables) yielding the best results (FIG. 2C).

The MMAI system substantially outperformed the NCCN risk stratification tool, encoded as a model, at predicting four key future outcomes for patients: distant metastasis, biochemical recurrence, prostate cancer-specific survival, and overall survival. By creating a deep learning architecture that simultaneously ingested multiple data types (of variable sizes) from a patient, as well as clinical data, a deep learning system capable of inferring long-term patient outcomes with substantially higher accuracy than established clinical models was built.

Methods and systems as described herein may leverage robust and large-scale clinical data from five different prospective, randomized, multinational trials with 10-20 years of patient follow-up for 5,654 patients across a varied population. Validation of these prognostic classifiers on a large amount of clinical trials data—in the intended use population—uniquely positions these tools as aids to therapeutic decision-making. Critical flaws of similar, genomics-based assays are their high costs and lengthy turnaround times; AI tools do not share these limitations, which substantially lower the barriers to large-scale international adoption. Nearly 60% of the world has access to the internet, yet only about 4% (the US population) have easy access to genomics-based assays. The growing adoption of digital histopathology, coupled with internet connectivity, may support global distribution of AI-based prognostic and predictive testing, enabling low-cost access to critical therapy personalization.

### Methods

Tabular Pipeline. The tabular clinical data was separated into numerical and categorical variables. Numerical variables were whitened (mean subtraction+max normalization) to the range [−1,1]. Categorical variables were treated as one-hot vectors that are embedded into 2-3 dimensional vectors following conventional word-to-vec techniques, with a dimensionality given by the formula D=Round(1.6·num_{c}ategories^{0.56}). A TabNet model that takes in a concatenation of categorical and numerical variables as input was used (parameters: learning rate 0.2, Adam optimizer with step learning rate scheduler, batch size of 1024, 50 max epochs with early stopping patience of 10 epochs).

Image Pipeline. A ResNet50 model, together with the MoCo-v2 training protocol (parameters: learning rate=0.03 with a cosine learning rate schedule, moco-t=0.2, multilayer perceptron head, batch size of 256, MoCo-v2 data augmentation, 200 epochs) was used to train the SSL models used in the system architecture of FIG. 2B. For each held-out test set in FIG. 3A, only the images of the training data were used to pre-train a corresponding SSL model. Certain image patches were oversampled using nucleic density sampling as described elsewhere herein. Once SSL pre-training was complete, all W×H patches were fed into the SSL pretrained ResNet50 model to generate a W×H×128 feature quilt for each image quilt. The final image model used for prediction was a 2-layer CNN model with batchnorm and dropout, which takes in the feature tensors as input. The final CNN model was trained with batch size of 32, 150 maximum epoch and Adam optimizer with learning rate of 0.01 and step learning rate scheduler.

Downstream Pipeline. A joint fusion approach was used to leverage information from both modalities (image and tabular features). The images were featurized to feature tensors and fed into the final image model to produce a feature vector, while the tabular features were separately fed into the TabNet model to produce another feature vector. Two fully-connected layers processed the concatenated feature vectors of each pipeline and output prediction probabilities. For patients with missing histopathology data, the image-based feature vector was zeroed prior to concatenation.

Dataset Preparation. Full patient-level baseline clinical data, digitized histopathology slides of prostate biopsies, and longitudinal outcomes from five landmark, large-scale, prospective, randomized, international clinical trials containing 5,654 patients, 16,204 histopathology slides, and 10-20 years of median patient follow-up was used. These trials were RTOG 9202, 9408, 9910, 0126, and 9413 (FIG. 2C). These trials randomized across various combinations of radiotherapy (RT) and androgen-deprivation therapy (ADT): RT+short-term ADT (RTS), RT+medium-term ADT (RTM), RT+long-term ADT (RTL), and dose and volume levels of RT (RT+). The slides were digitized over a period of two years by NRG Oncology using a Leica Biosystems Aperio AT2 digital pathology scanner at a resolution of 20x. The histopathology images were manually reviewed for quality and clarity. Digital slides were converted into a single image quilt of size 200 by 200 patches for each unique patient prior to model training. Each clinical trial collected slightly different clinical variables. Six clinical variables that were available across all trials (combined Gleason, Gleason primary, Gleason secondary, t-stage, baseline PSA, age), along with the digital histopathology, were used for model training and validation.

Tissue segmentation. After slicing the slides into 256×256-pixel patches at 10× zoom, developed an artifact classifier was developed by training a ResNet-18 to classify whether a patch showed usable tissue, or whether it showed whitespace or artifacts. The artifact classifier was trained for 25 epochs, optimized using SGD with a learning rate of 0.001. The learning rate was reduced by 10% every 7 epochs. 3661 patches (tissue vs not tissue) were manually annotated, and the classifier was trained on 3366 of them, achieving a validation accuracy of 97.6% on the remaining 295. This artifact classifier was then used to segment tissue sections during image quilt formation.

Nucleic Density Sampling. Due to significant variation in stain intensity and stain degradation, readily-available pretrained models for nuclei detection and segmentation were unable to accurately detect nuclei in a majority of our slides. To overcome this, a nuclei detector was trained using the YOLOv5 (github.com/ultralytics/yolov5) object detection method.

In order to train the YOLOv5 model, a representative sample of 34 handpicked slides were manually labeled using the QuPath image analysis platform. First, the “Simple tissue detection” module was used to segment tissue. Next, the “Watershed cell detection” module was used to segment cells, with manually tuned parameters for each slide. A YOLOv5-Large model was then trained on the annotations from 29 of the slides and evaluated on the remaining 5. This model was trained using 256×256 patches at 10× zoom.

Model Performance Metrics. (AUC) For each model and each outcome, the time-dependent receiver operating characteristic, accounting for competing events, was estimated using the R-package timeROC. This is a curve of time-dependent sensitivities and specificities computed by sweeping a threshold t in the interval [0,1] and defining a model's prediction as ŷ=P>t, where P is the outcome probability outputted by the model. The area under this curve defines the model's performance on the task at hand.

NCCN Model. The NCCN model was coded according to the algorithm in FIG. 8, using three clinical variables—Gleason, t-stage, and baseline PSA—to bin patients into low, medium, and high-risk groups.

### Example 2: Evaluating Algorithmic Fairness of MMAI Algorithms

In this Example, the algorithm fairness, or the performance of a multi-modal AI (MMAI) models utilizing clinical and digital histopathology data in AA and non-AA prostate cancer patients treated on NRG/RTOG prostate cancer trials, is described.

Prostate cancer (PCa) is the second leading cause of cancer-related mortality in men, and it is well established that African American (AA) men experience an increased burden of disease due to more advanced presentation and younger age at diagnosis.

There have been limitations associated with studying prognostic outcomes and associated disparities among AA men with PCa using population-based data sets and retrospective studies because these data often do not offer adequate representation of AA men within the cohorts and lack long-term follow-up outcomes required for developing prognostic risk models. Ideally, studies including large numbers of AA men participating in prospective randomized controlled trials (RCTs) allow for evaluation of long-term prognostic outcomes using a representative AA sample while minimizing the risk for selection bias and other co-founders. The Radiation Oncology Group (RTOG) and the NRG Oncology cooperative group have prioritized recruitment of representative proportions of AA onto prostate cancer trials. Large-scale RCTs offer an opportunity for modeling long-term prognostic outcomes and differences in risk that can lead to more nuanced clinical decision-making for treatment selection and therapeutic optimization among AA men with PCa.

### Methods

### Description of Multi-Cohort Data

With permission from NRG Oncology, a National Clinical Trials Network (NCTN) group funded by the National Cancer Institute (NCI), a unique dataset was assembled from five large multinational, randomized phase III clinical trials of men with localized prostate cancer (NRG/RTOG-9202, 9408, 9413, 9910, and 0126). All patients received definitive external radiotherapy (RT), with or without pre-specified use of androgen deprivation therapy (ADT). Combined RT with short-term ADT had a duration of 4 months, medium-term ADT had a duration of 36 weeks, and long-term ADT had a duration of 28 months. In total, there were 7,752 eligible participants randomized to these five trials.

### Description of the Multimodal AI (MMAI) Model

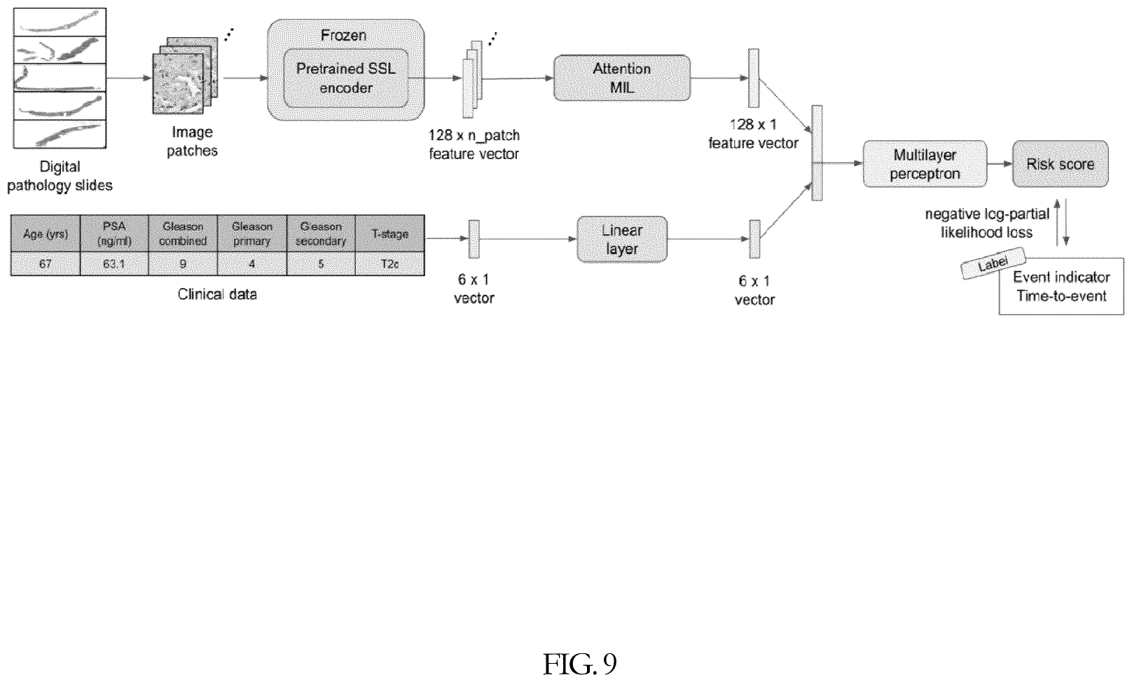

Four MMAI models as described herein were trained and deployed on the dataset. The MMAI models jointly learned the relevant features from the digital histopathology slides and clinical data from each patient. Image vector representations were learned and extracted from the tissue sections in the biopsy slides through self-supervised pre-training. A combination of these image feature vectors and feature vectors derived from clinical data was fed into a multimodal fusion pipeline to output a risk score for the desired clinical endpoints including distant metastasis (DM) and prostate cancer-specific mortality (PCSM). The cohort was split into an 80/20 development and validation datasets, where the MMAI model was trained and optimized on the development set and subsequently validated on the remaining validation set. The first MMAIs predicting risk of DM and PCSM were as described in Example 1. The second MMAI models predicting risk of DM and PCSM comprised multimodal learning based on a multiple instance learning-based neural network with an attention mechanism using the time to event of the desired clinical endpoints as labels, as described in more detail herein below. A schematic overview of the second set of MMAI models is shown in FIG. 9. A comparison of study findings based on the MMAI models as described in Example 1 is also discussed below.

### Methods for Multi-modal Deep Learning Model Development

### Model Development Overview

The five trials were stratified by 1) trial, 2) status of distant metastasis, and 3) patient clinical risks, and randomly split into development (80%) and validation (20%) sets for model development and validation, respectively. Each MMAI model was trained and optimized on the development set through a 5-fold cross-validation scheme, where the development set was further split into training and tune subsets in each fold. The training subset was used to update learnable model parameters, whereas the tune subset was used to monitor unbiased performance during training and to tune hyperparameters. As this training process generated five separate models, an ensemble model was then constructed by taking an average across the five model outputs to form a single risk score for each patient.

### Clinical Data Preprocessing

Clinical variables (T-stage, Gleason score, and primary/secondary Gleason pattern) were all treated as numerical variables and were standardized based on the mean and standard deviation of the training data. Any missing clinical data was imputed with a k-Nearest Neighbors method, where missing values are imputed using the mean value from 5 nearest neighbors found in the training set.

### Image Feature Extraction Model Development

Effective learning of relevant features from a variable number of digitized histopathology slides involves both image standardization and self-supervised pre-training. For each patient, all the pre-treatment biopsy tissue sections in their biopsy slides were segmented out and divided them patches of size 256×256 pixels across each respective RGB channels. A tissue classifier was developed by training a ResNet-18 to classify whether a patch showed usable tissue or whether it showed whitespace or artifacts. The artifact classifier was trained for 25 epochs, optimized using stochastic gradient descent with a learning rate of 0.001. The learning rate was reduced by 10% every 7 epochs. 3661 patches (tissue vs. not tissue) were manually annotated, and the classifier was trained on 3366 of them, achieving a validation accuracy of 97.6% on the remaining patches. This artifact classifier was then used to segment tissue sections and filter out low-quality images during image feature generation.

Patches filtered by the artifact classifier were then used to train a self-supervised learning model to learn histomorphological features useful for downstream tasks. A ResNet-50 model was used together with the MoCo-v2 training protocol (parameters: learning rate=0.03 with a cosine learning rate schedule for 200 epochs, moco-t=0.2, multilayer perceptron head, batch size of 256, the default MoCo-v2 parameters for augmentation) to train the self-supervised learning model. Images of patients with a Gleason primary ≥4 were used to pre-train a corresponding self-supervised learning model to effectively learn relevant histomorphological features. Once self-supervised pre-training was complete, all patches with usable tissue of a whole-slide image were fed in to the self-supervised pretrained ResNet-50 model to generate a 128-dimensional vector representation for each patch.

### Downstream Multi-Modal Prognostic Model Development

The downstream prognostic model took the image feature tensor, a concatenation of feature vectors from all patches for each patient, and preprocessed clinical data as input for each patient. In the second model, an attention multiple instance learning network was employed to learn a weight for each image feature vector from each patch. A single 128-dimensional image vector was generated from the image feature tensor for each patient by taking the weighted sum of the image vectors of all patches from the same patient, where the weights were learned by the attention mechanism. The preprocessed clinical data were all considered as numerical variables and processed through a single linear layer to learn a 6-dimensional clinical vector representation. A concatenation of the 128-dimensional image vector and the 6-dimensional clinical vector was further processed through the neural network-based joint fusion pipeline to effectively learn from both clinical and image data to output risk scores for an outcome of interest (FIG. 9).

Negative log-partial likelihood was employed as the training objective, where model prediction scores were the estimated relative log hazards. A binary indicator for an event of interest and a corresponding time to event were used as labels for model development. The negative log-partial likelihood loss was parameterized by the model weights θ and formulated as follows:

$$loss ( θ ) := - \frac{1}{{N}_{E = 1}} {\sum }_{i : {E}_{i} = 1} ( {f}_{θ} ( {x}_{i} ) - log {\sum }_{j ∈ R ( {T}_{i} )} {e}^{{f}_{θ} ( {x}_{j} )} )$$

where the values Tᵢ, Eᵢ, and xᵢ are the event time or time of last follow-up, an indicator variable for whether the event is observed, and the model input for the ith observation, respectively. The function f_{θ} represents the factual branch of the multi-modal model, and f_{θ}(x) is the estimated relative risk given an input x. The value N_{E=1} represents the number of patients with an observable event. The set of patients with an observable event is represented as Eᵢ=1. The risk set [symbol] (t)={i:Tᵢ≥t} is the set of patients still at risk of failure at time t. Breslow's approximation was used for handling tied event times.

### Metrics of Model Performance in Subgroups

Distant metastasis (DM), prostate cancer-specific mortality (PCSM), biochemical failure (BF), and overall survival (OS) were evaluated. DM and PCSM were chosen because they are strongly correlated with prostate cancer morbidity and mortality and likely represent a more clinically useful measure to reflect the broader burden of prostate cancer for the AA compared to non-AA population.

Model performance between AA and non-AA PCa patients of DM MMAI against the DM endpoint and PCSM MMAI against PCSM endpoint was evaluated for each MMAI model. Patients with unknown or missing race status were excluded from the analysis cohort. All evaluated endpoints were time-to-event outcomes where patients lost to follow-up were censored and deaths before experiencing the event of interest were considered as competing events. The racial subgroup analyses were conducted by comparing distributions of clinical variables and MMAI scores (medians and interquartile range (IQR) for continuous variables and proportions for categorical variables reported) and evaluating MMAI models' prognostic ability among the AA and non-AA men. Both MMAI continuous scores (per 0.05 score increase) and categorized risk groups were used to assess the algorithm fairness. For the MMAI categorical groups, the model scores were ranked by deciles and then collapsed into three groups by binning the deciles with similar prognosis based on the corresponding endpoint that the MMAI model was originally trained for. For example, the DM MMAI model was grouped as 1-4th, 5-9th, and 10th decile, and the PCSM MMAI model was grouped as 1-5th, 6-9th, and 10th decile. The performance of the models was compared using DM and PCSM as the primary endpoint and secondary endpoints of BF, OS with Fine-Gray or Cox Proportional Hazards models. Either Kaplan-Meier or cumulative incidence estimates were computed and compared using log-rank or Gray's test. The p-values were then adjusted post hoc using the Bonferroni method for the pairwise cumulative incidence comparisons between the subgroups.

### Results

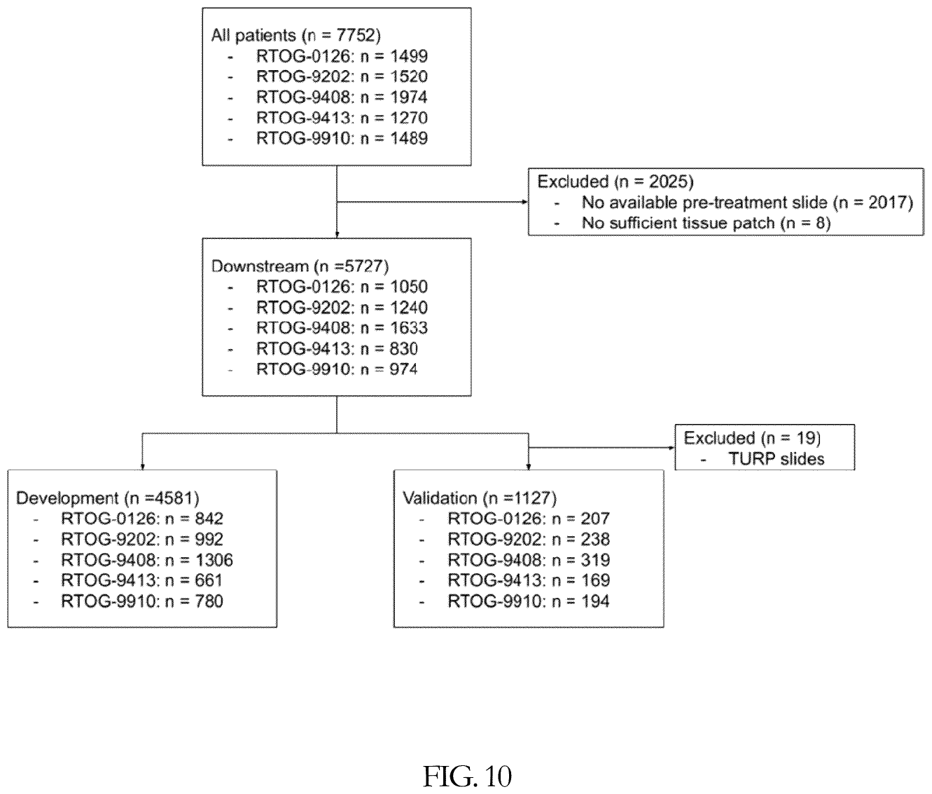

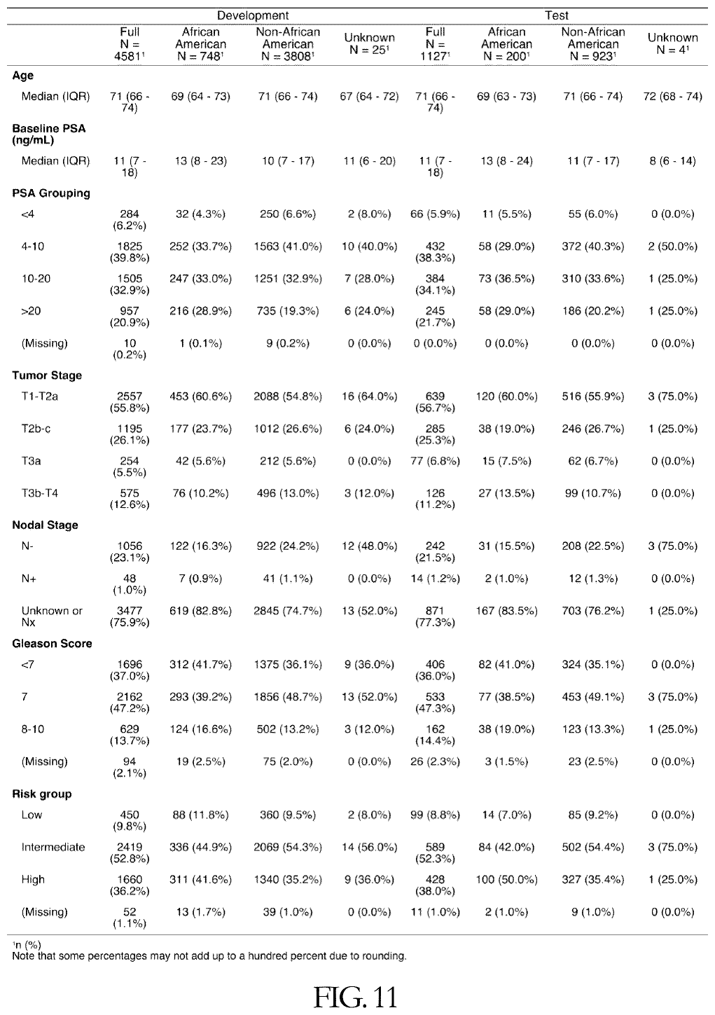

A schema of the pooling of eligible clinical trial participants is depicted in FIG. 10. There was a total of 948 (16.6%) AA patients, 4,731 non-AA patients (82.9%), and 29 (0.5%) patients with unknown or missing race status, and these 29 patients were excluded from all the analyses.

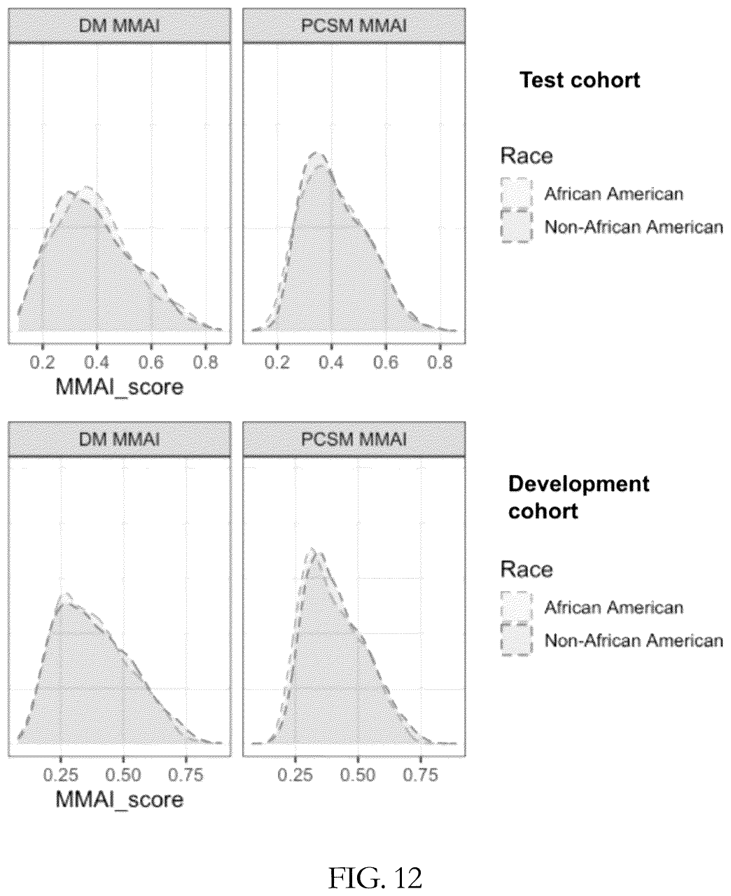

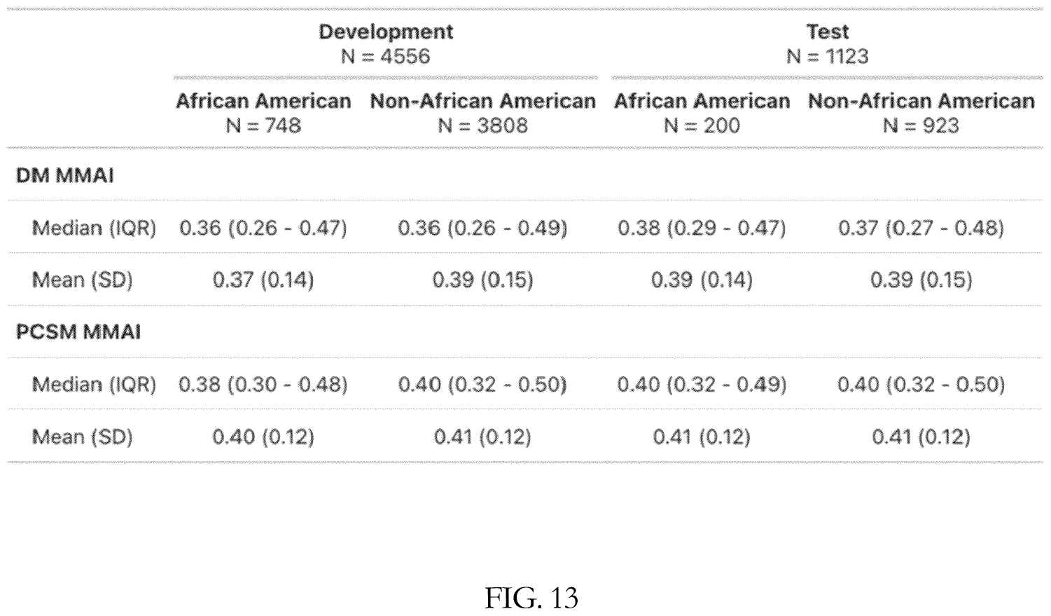

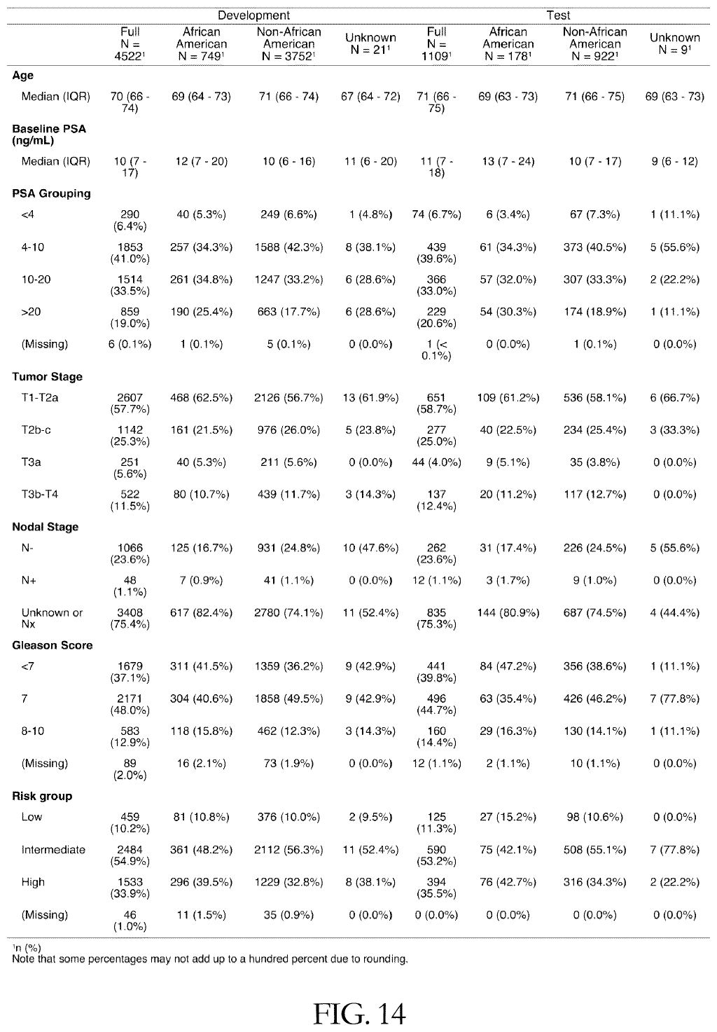

In both the development and test cohorts, AA and non-AA patients had a median age of 69 vs. 71 years old, respectively. In the development cohort, compared to non-AA patients, AA had a higher median baseline PSA (13 vs. 10 ng/mL), more T1-T2a (61% vs. 55%), more Gleason 8-10 (17% vs. 13%) and more National Comprehensive Cancer Network (NCCN) high risk (42% vs. 35%); Similar findings were observed in the test cohort with the exception of T Stage (FIG. 11). For all MMAI models in both development and test cohorts, the distributions overlap between AA and non-AA subgroups (FIG. 12). The median (IQR) score of the model optimizing for DM (DM MMAI) was 0.36 (0.26-0.47) in AA and 0.36 (0.26-0.49) in non-AA in development cohort, and 0.38 (0.29-0.47) vs 0.37 (0.27-0.48) in test cohort. The median score for DM MMAI was 0.38 (0.30-0.38) in AA and 0.40 (0.32-0.50) in non-AA in development cohort, and 0.40 (0.32-0.49) vs 0.40 (0.32-0.50) in test cohort (FIG. 13). Findings for the first MMAI model are reported in FIG. 14.

### Performance of Multimodal AI (MMAI) Models in Subgroups

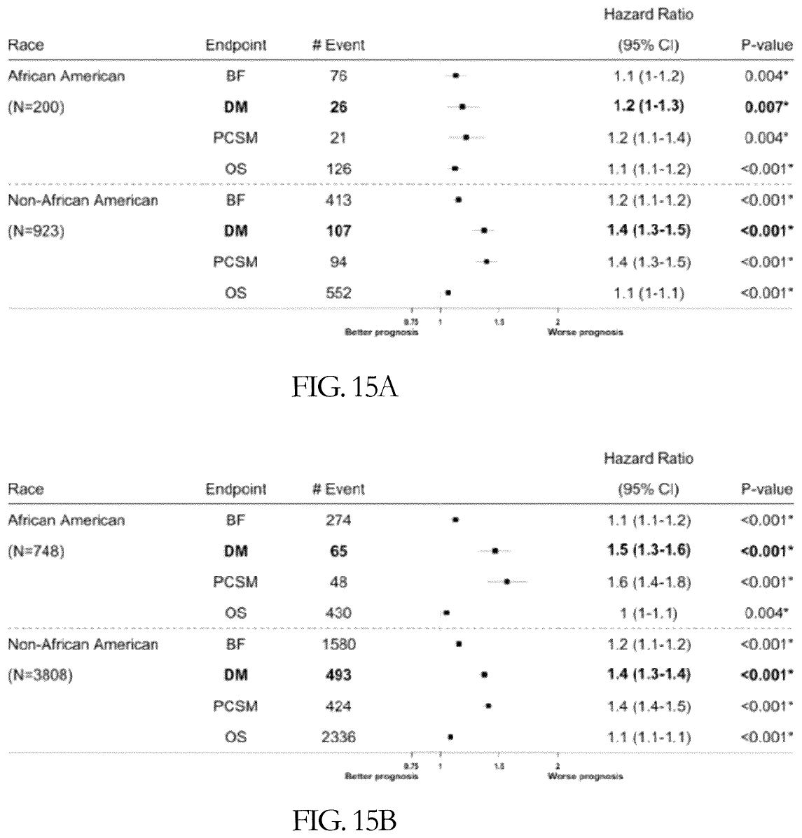

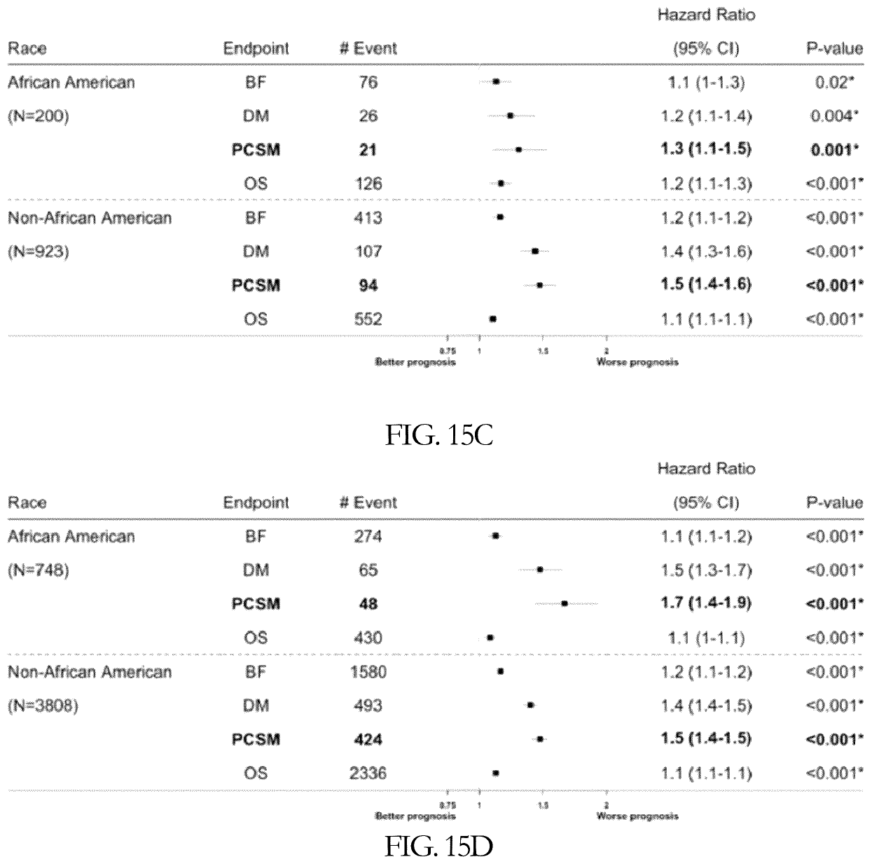

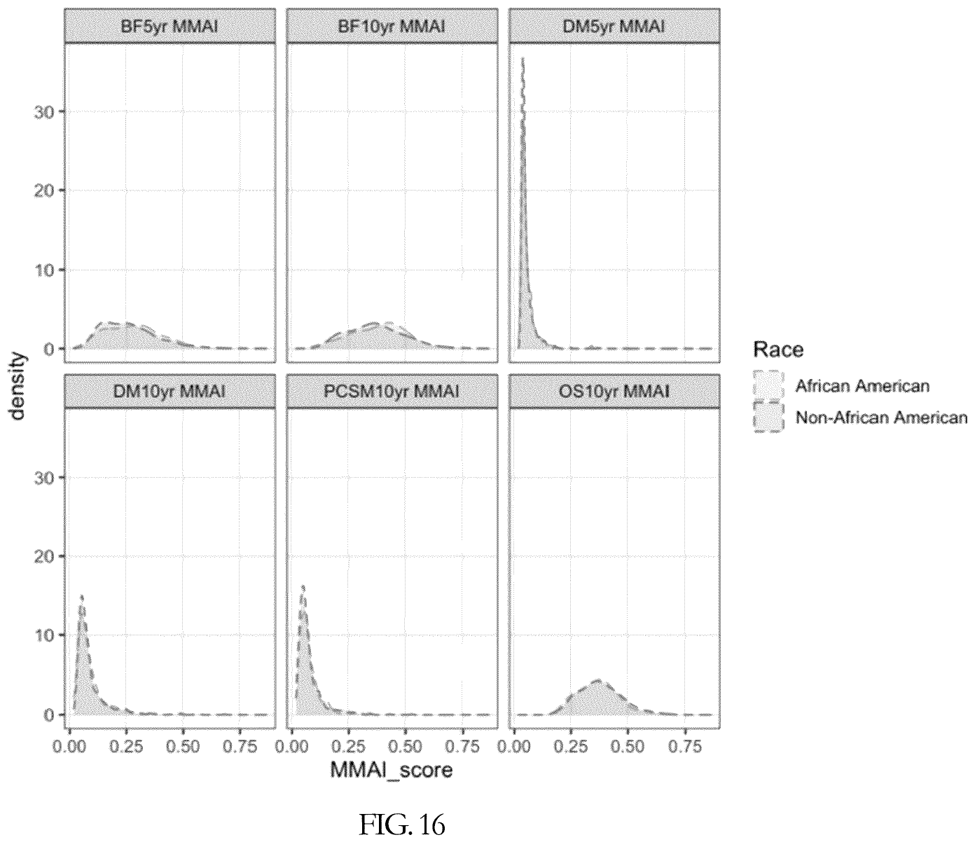

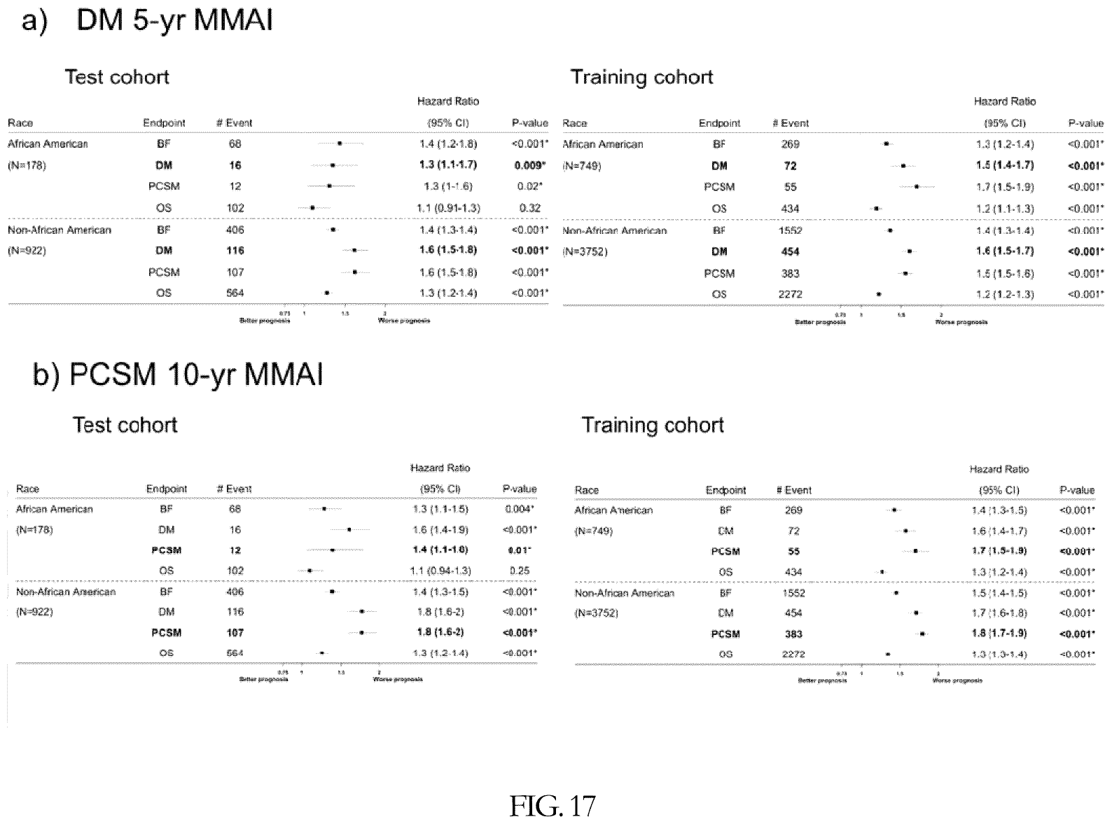

In the test cohort, the DM MMAI model score showed a strong prognostic signal for DM in both the AA (hazard ratio [HR]per 0.05 score increase: 1.2 for DM, p=0.007) and non-AA subgroup (1.4 for DM, p<0.001) (FIG. 15A). Similarly, the PCSM MMAI score showed a strong prognostic signal for PCSM in both AA (HR per 0.05 score increase: 1.2 for PCSM, p=0.01) and non-AA subgroup (1.5 for PCSM, p<0.001) (FIG. 15B). All original models demonstrated similar results in both AA and non-AA subgroups (FIG. 16 and FIG. 17).

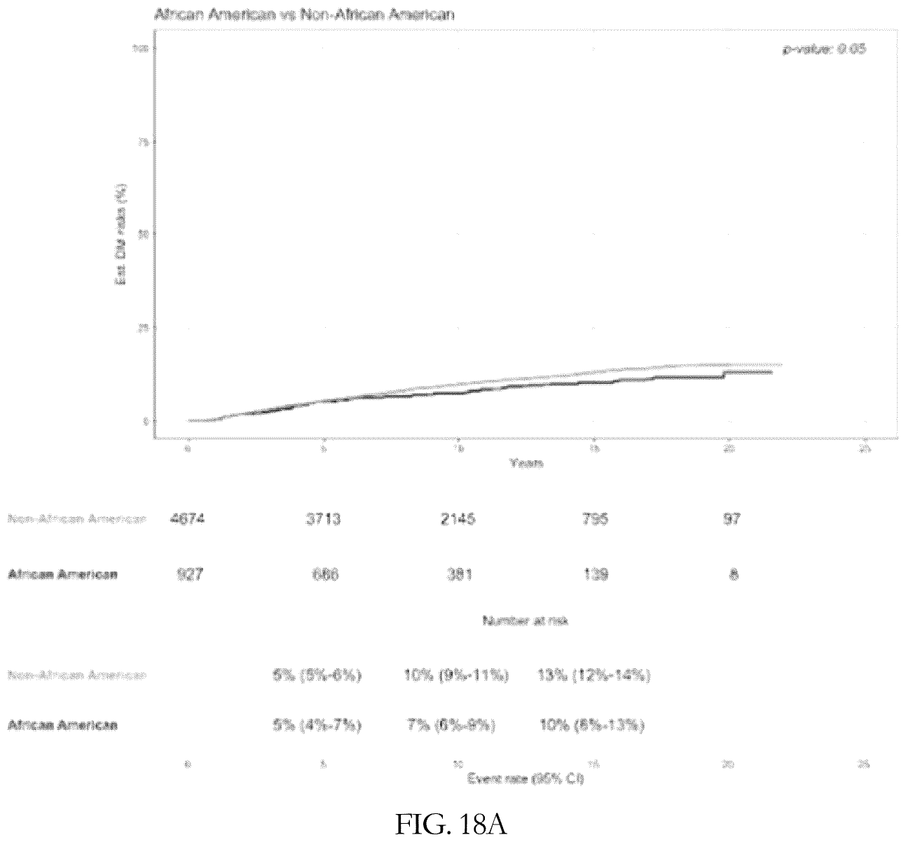

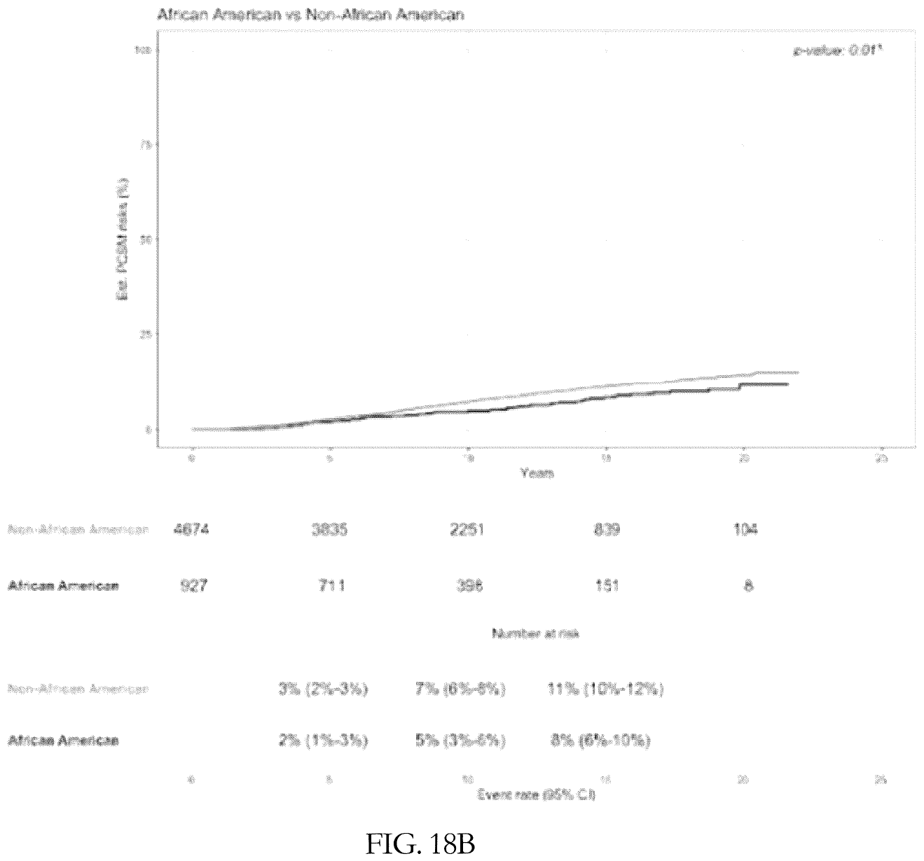

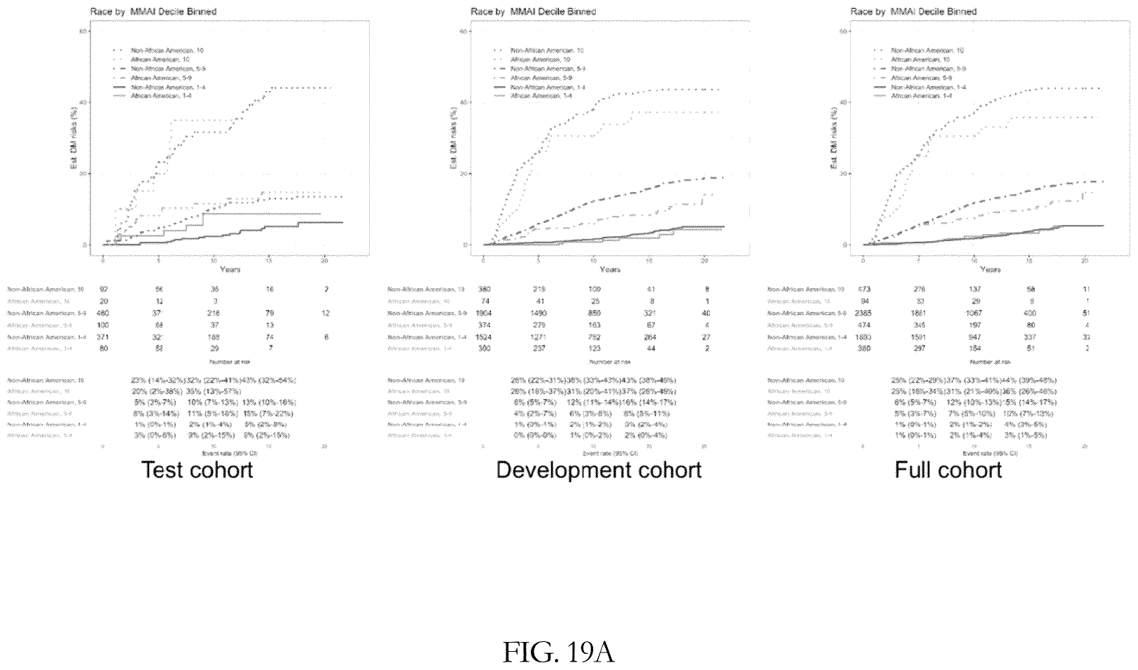

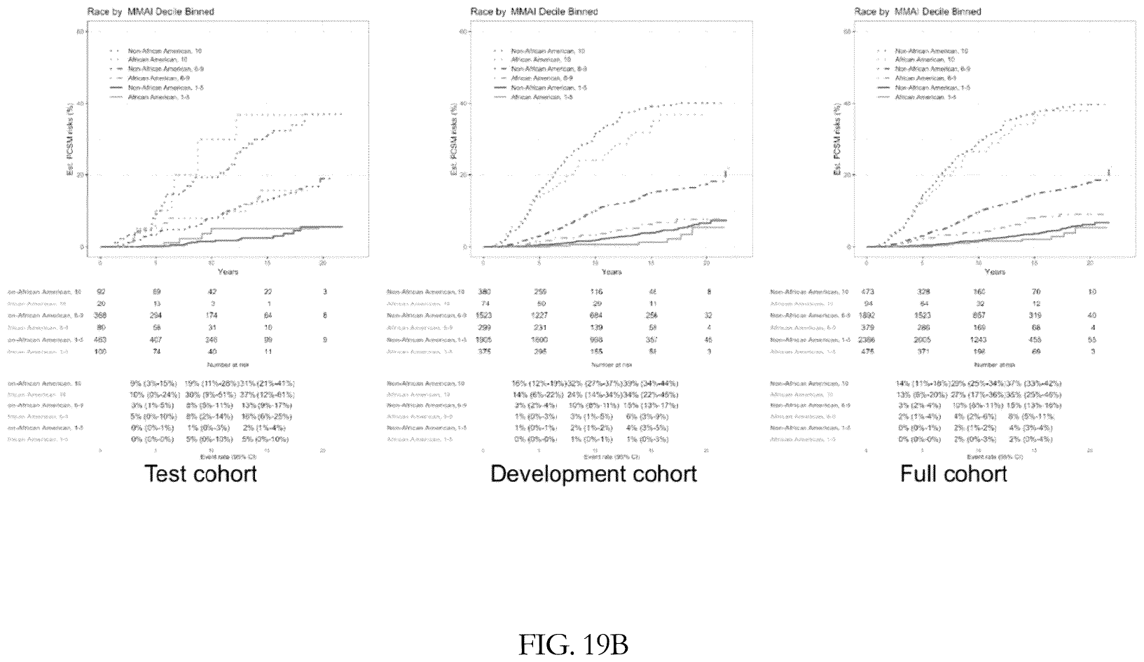

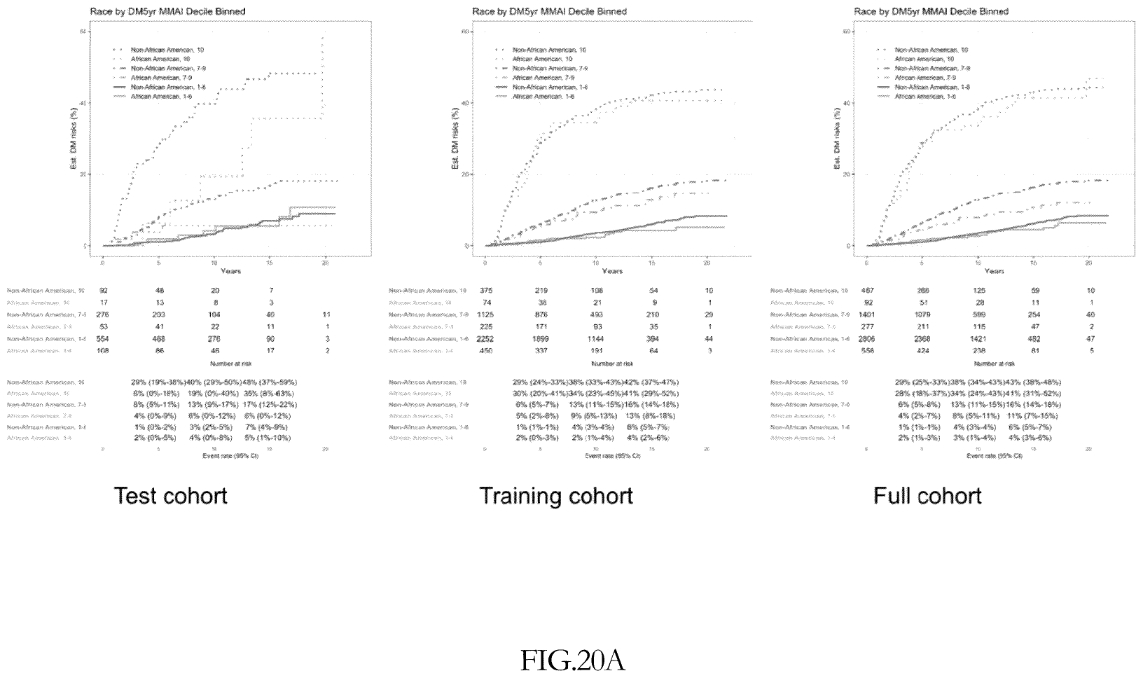

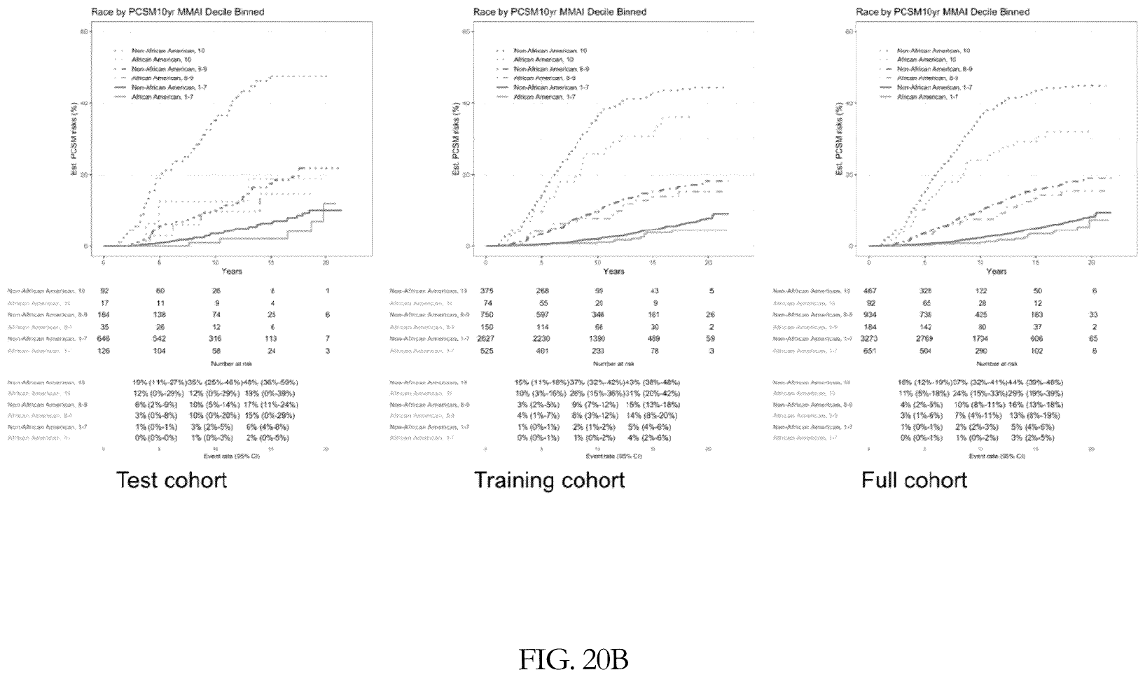

Racial subgroups' cumulative incidences were compared in the full cohort; at 10-year, the estimated DM rate was 5% (3%-6%) for the AA subgroup and 7% (6%-8%) for the non-AA subgroup (FIG. 18A). Both MMAI models were able to risk-stratify patients within the AA subgroup and within the non-AA subgroup (FIGS. 19A and 19B). In the test cohort, for the DM MMAI model, the 5-yr estimated DM rate for the AA subgroup was 3% (95% CI: 0%-6%), 8% (95% CI: 3%-14%), and 20% (95% CI: 2%-38%) and for the non-AA subgroup was 1% (95% CI: 0%-1%), 5% (95% CI: 3%-7%), and 23% (95% CI: 14%-32%). The adjusted pairwise comparisons between AA and non-AA for different risk groups were not statistically significant (p-values=0.36, 1.00, 1.00, respectively). Similarly, for the PCSM MMAI model, the 10-yr estimated PCSM rate was 5% (95% CI: 0%-10%), 8% (95% CI: 2%-14%), and 30% (95% CI: 9%-51%) for the AA subgroup, and 1% (95% CI: 0%-3%), 8% (95% CI: 5%-11%), and 19% (95% CI: 11%-28%) for the non-AA subgroup (FIG. 18B). The adjusted pairwise comparisons between AA and non-AA for different risk groups were not statistically significant (p-values=1.00, 1.00, 1.00, respectively). The original MMAI models showed similar results for both models in both AA and non-AA subgroups (FIGS. 20A and 20B).

### Discussion

AI-based biomarkers can help physicians tailor treatment recommendations for patients with prostate cancer. However, AA men may be underrepresented in population data used to develop novel biomarkers. Previous biomarker studies have raised questions as to their value when developed in largely non-AA cohorts and then applied to AA men. This paucity of genomic data inclusive of AA populations has the potential to exacerbate the known health disparities experienced by this population by the algorithmic encoding of these inequalities. These observations underscore the need to examine the performance of biomarkers across racial lines with the use of more clinically relevant endpoints and the application of rigorous methods to control for selection biases.

Substantial data was used to train the MMAI models, and the prognostic performance of the AI models was found to be comparable between AA and non-AA subgroups. DM and PCSM MMAI models performed similarly in both the AA and the non-AA patient population, demonstrating algorithmic fairness in the application of our tools. This approach supports the use of these AI biomarkers to personalize treatment choices for men with prostate cancer across racial groups. Additionally, this analysis offers an approach for integration of algorithmic fairness principles in routine biomarker discovery and validation.

### Example 3: Risk Stratification of Prostate Cancer Patients Using MMAI

In this example, the ability of multi-modal artificial intelligence (MMAI) models as described herein to stratify patients into risk groups was compared to that of the National Comprehensive Cancer Network (NCCN) risk stratification schema.

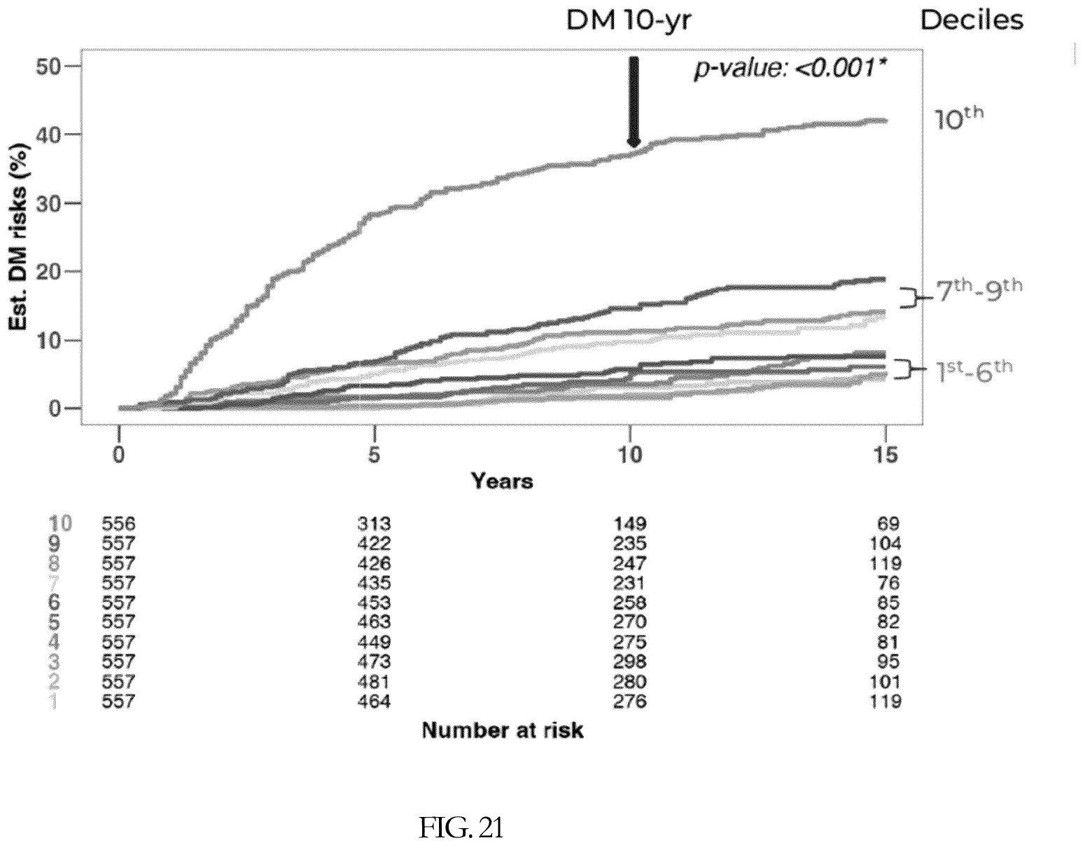

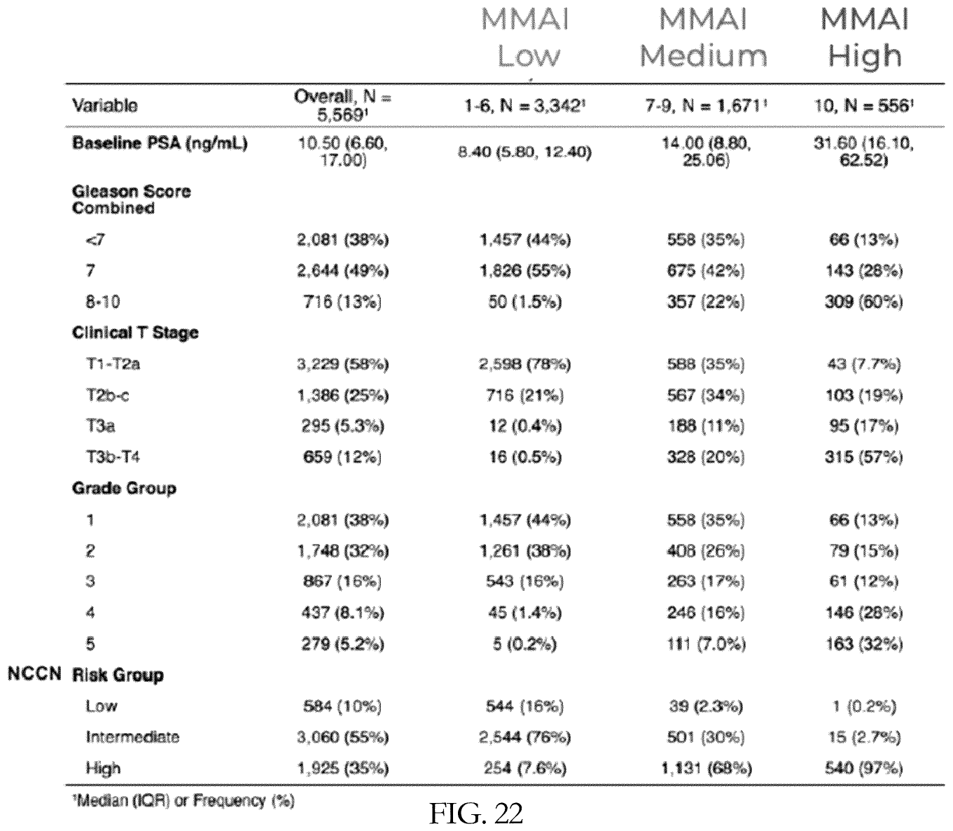

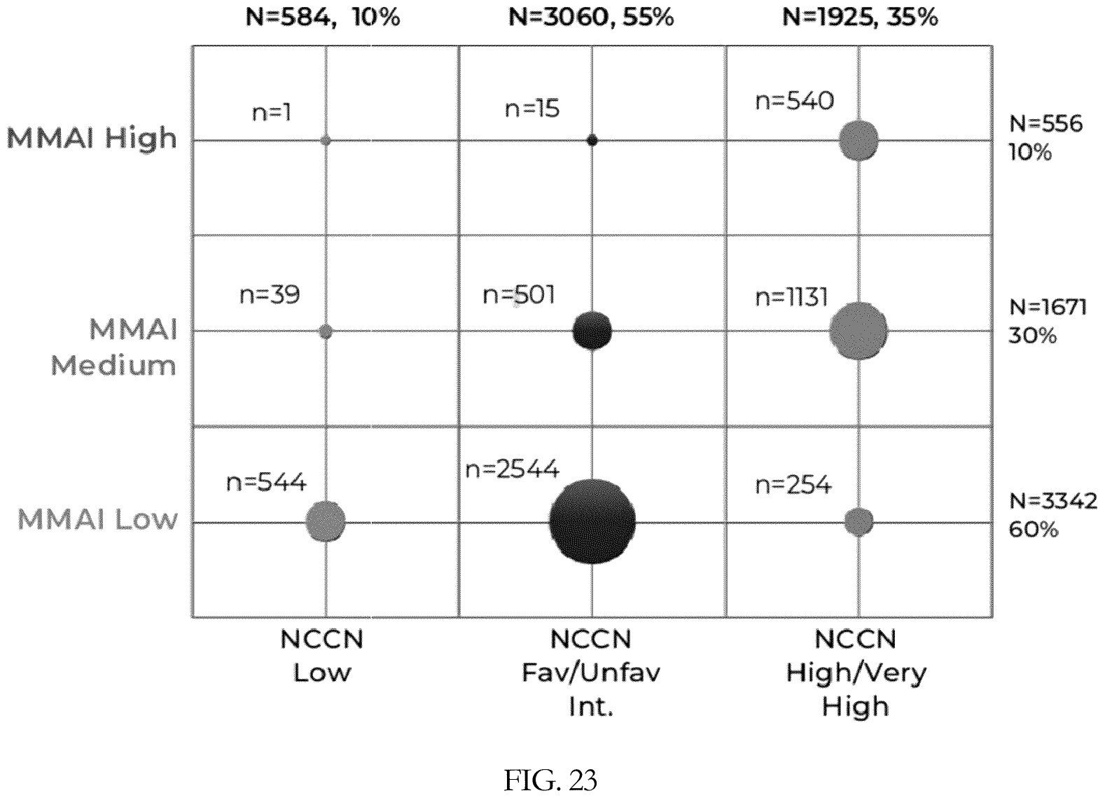

5,569 individuals from the dataset described in Example 1, for whom a definitive NCCN risk classification could be made, were sorted into one of ten deciles based on 10-year risk of distant metastasis (DM 10-yr) according to corresponding MMAI score as predicted by an MMAI model as described in Example 2. Each decile was then stratified into one of three MMAI prognostic risk groups, “MMAI Low,” “MMAI Medium,” and “MMAI High,” based on MMAI DM 10-yr score (<10%, 10%-25%, and >25%, respectively) (FIG. 21). Baseline characteristics of each MMAI prognostic risk group are shown in FIG. 22. FIG. 23 depicts per MMAI prognostic risk group (rows), which number of individuals were classified according to the NCCN risk schema as “Low,” “Intermediate” (favorable and unfavorable), or “High” (NCCN High or Very High).

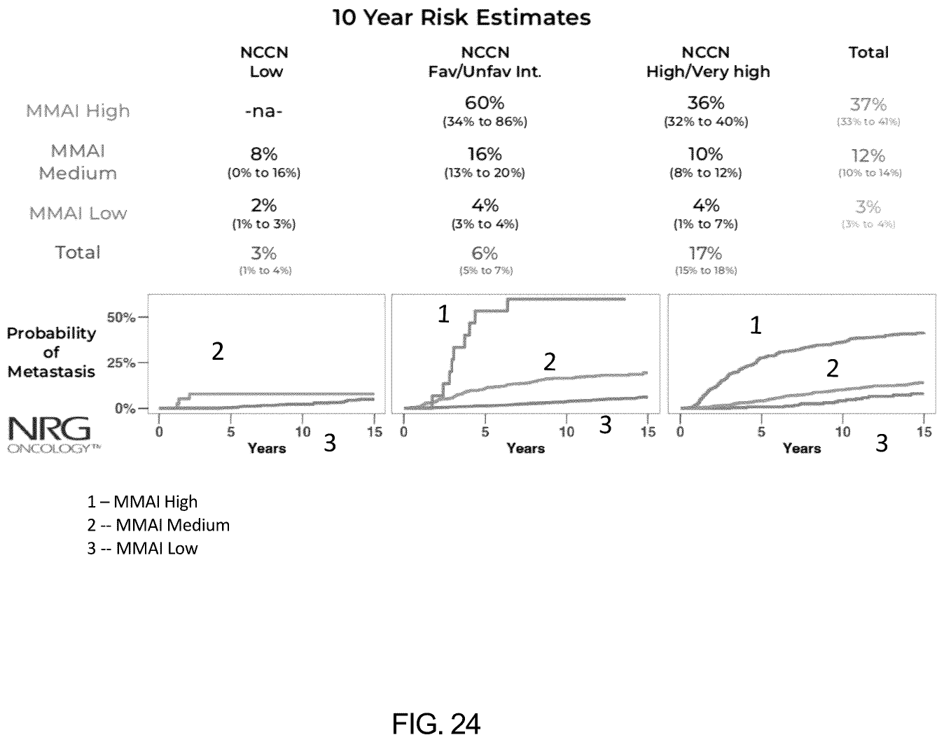

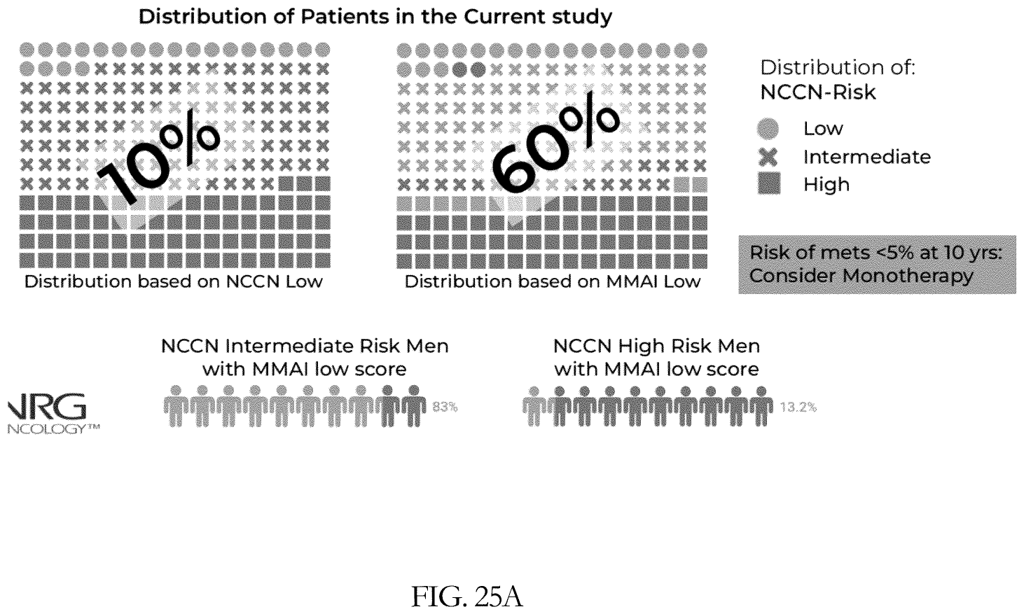

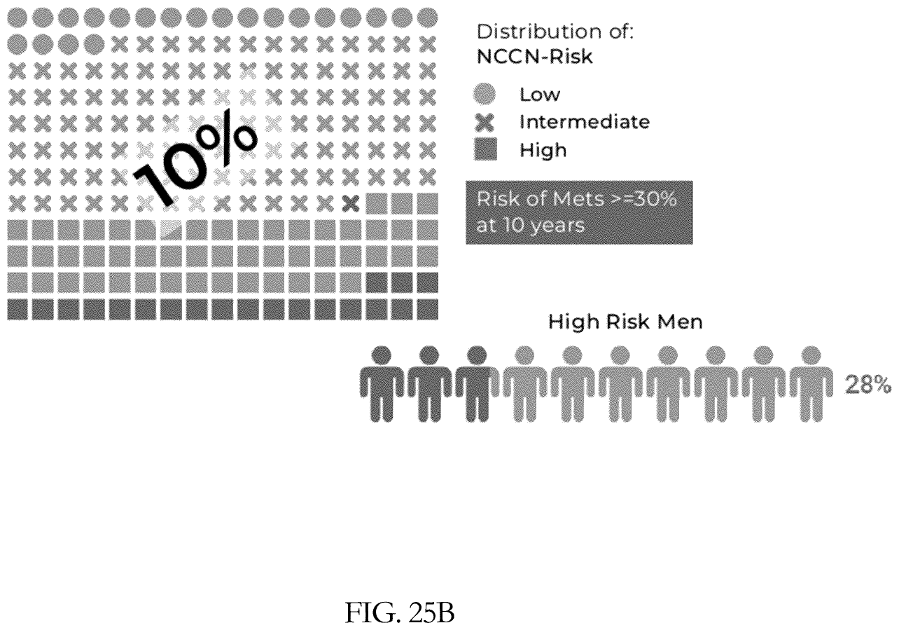

For each individual, probability of DM 10-yr was again calculated using the MMAI model to determine if MMAI could better prognosticate distant metastasis at 10 years than the NCCN risk classification. FIG. 24 shows the average risk of DM 10-yr (confidence interval in parentheses) for individuals with a given NCCN and MMAI classification. As shown in FIG. 24, the risk of DM 10-yr is roughly the same for individuals whether classified as low risk by NCCN or MMAI. However, the MMAI model is better able to determine which individuals classified by the NCCN scheme as either intermediate or high risk are actually at an elevated risk for DM. As shown in FIG. 24, the subset of individuals classified as NCCN “intermediate” that were classified as MMAI “high” had a 60% MMAI predicted probability of DM 10-yr while the subset of individuals classified as NCCN “high” that were classified as MMAI “high” had a 36% MMAI predicted probability of DM 10-yr. Thus, the MMAI-based risk classification could stratify NCCN intermediate those individuals with a risk of metastasis. As illustrated in FIG. 25A, the MMAI model identified 6-fold more patients than NCCN with the lowest risk of metastasis. Across those individuals with an NCCN “intermediate” risk, about 83% had an MMAI low score and thus a low risk of metastasis while across those with an NCCN “high” risk, about 13.2% had an MMAI low score. Across those individuals classified as “high” risk by NCCN, about 28% had a “high” MMAI DM 10-yr score (risk ≥30%) (FIG. 25B).

Compared to the NCCN classification, MMAI systems as disclosed herein can thus better stratify those individuals at risk of prostate cancer metastasis.

### Example 4: External Validation of MMAI Models

This example describes the validation of multimodal artificial intelligence (MMAI) models for prognosticating prostate cancer risk as described herein.

### Patients

NRG/RTOG-9902 enrolled 397 high risk, localized prostate cancer (PCa) patients who were randomized to receive long term androgen suppression (AS) with radiotherapy (RT) alone (AS+RT) or with adjuvant combination chemotherapy (CT) (AS+RT+CT) between January 2000 to October 2004. CT was four 21-day cycles with paclitaxel, estramustine, and oral etoposide delivered beginning 28 days following 70.2 Gy RT. The AS regimen was luteinizing hormone-releasing hormone (LHRH) for 24 months beginning 2 months prior to RT plus oral anti-androgen for 4 months before and during RT. Men were enrolled if they had either PSA between 20-100 and Gleason Score ≥7 or clinical stage ≥T2 and Gleason score ≥8. Ten-year results showed no statistically significant difference in overall survival (OS), biochemical failure (BF), local progression (LP), distant metastasis (DM), or disease-free survival (DFS) between the two treatment arms (p>0.05 for all endpoints listed). As such, in the present example all men were pooled into one cohort regardless of treatment arm.

Sample process and Scanning

Pre-treatment biopsy slides from RTOG-9902 were digitized by NRG Oncology using a Leica Biosystems Aperio AT2 digital pathology scanner at 20× resolution. The histopathology images were reviewed for quality and clarity by the NRG Biobank operator and by the artificial intelligence data intake team. A previously built artifact classifier was used to filter out low-quality images.

### Description of the Multimodal AI (MMAI) Model

The MMAI architecture with 6 MMAI algorithms was developed and validated using five phase III NRG trials (RTOG 9202, 9408, 9413, 9910, 0126) utilizing both digital histopathology slides and clinical data from each patient, as described in Example 2 and illustrated in FIG. 9. From this MMAI architecture, there were two locked MMAI algorithms risk scores optimized for the desired clinical endpoints—distant metastasis (DM) and prostate cancer specific mortality (PCSM).

### End Points

Primary endpoints for this study were (1) DM, defined days from randomization to date of distant metastasis, and (2) time to PCSM defined as days from randomization to date of death from prostate cancer.

Secondary endpoints were time to biochemical failure (BF) defined as days from randomization to date of biochemical failure (the first of either prostate-specific antigen (PSA) failure or the initiation of salvage hormone therapy), and time to overall survival (OS) defined as days from randomization to date of death.

Subjects who were lost to follow-up before experiencing an event of interest were censored at their last follow-up. For DM, PCSM and BF, death before experiencing the event of interest was considered as competing events.

### Statistical Analysis

Digital pathological evaluable population (DPEP) was defined as subjects who were randomized to RTOG-9902, with quality histopathology data and no missing baseline clinical variables including age, clinical T-stage, primary and secondary Gleason grade, and baseline PSA to generate MMAI algorithm scores for analysis.

The baseline demographic and clinical characteristics were summarized descriptively for the DPEP and intention to treat (ITT) population and compared between the DPEP and the subgroup of patients from the ITT but without quality histopathology data. Descriptive summaries were provided using count and portion (%) for categorical variables, median and interquartile range (IQR) for continuous variables. P-values were calculated using Wilcoxon rank sum test for continuous variables, and Pearson's Chi-square test or Fisher's exact test for categorical variables.

The prognostic performance of the MMAI algorithms were assessed using univariable and multivariable analyses. The Fine and Gray regression was used to estimate subdistribution hazard ratio (sHR) and 95% confidence interval (CI) for DM, PCSM and BF endpoint. The Cox Proportional Hazards regression was used to estimate HR and 95% CI for OS endpoint. The MMAI algorithm scores were split by quartiles and summarized using cumulative incidence curves with five- and ten-year estimated DM and PCSM rates and corresponding two-sided 95% CIs provided. The tests for MMAI-treatment interaction were also performed as exploratory analysis.

All statistical analyses were performed using R, version 4.1.2 (R Foundation for Statistical Computing, Vienna, Austria). All statistical tests were 2-sided and used a 0.05 significance level. The prognostic model validation findings were reported using the TRIPOD reporting criteria.

### Results

### Participants

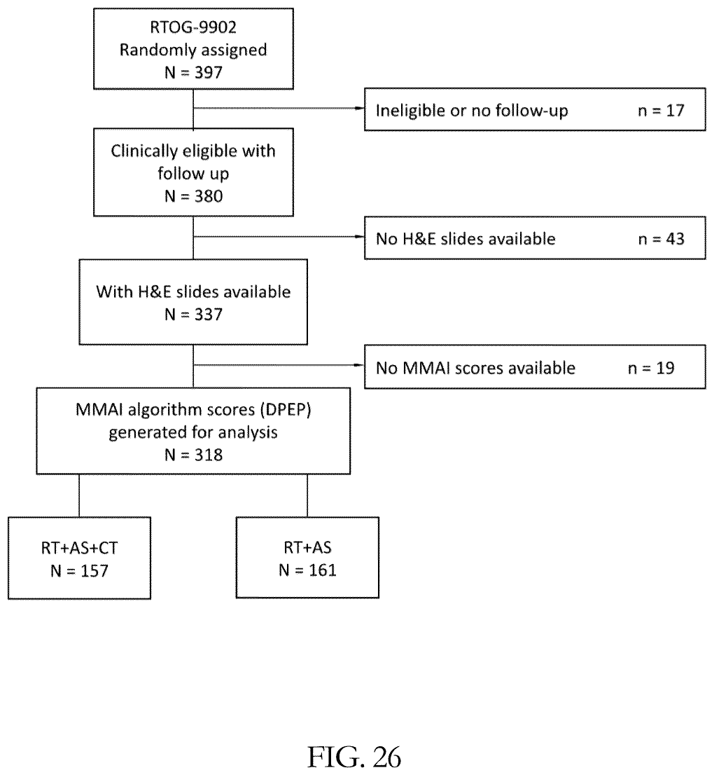

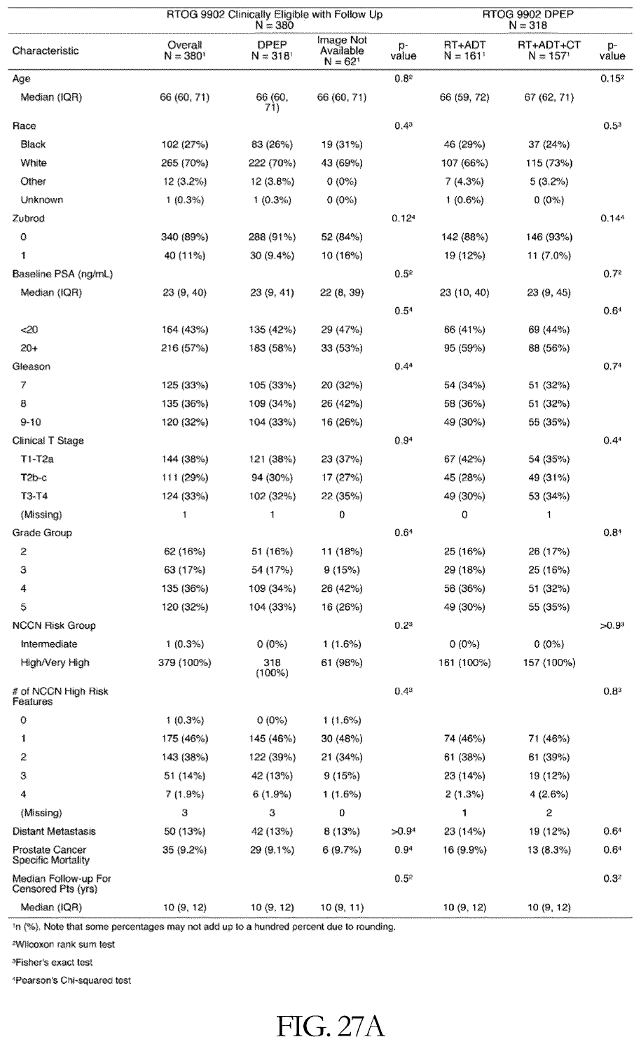

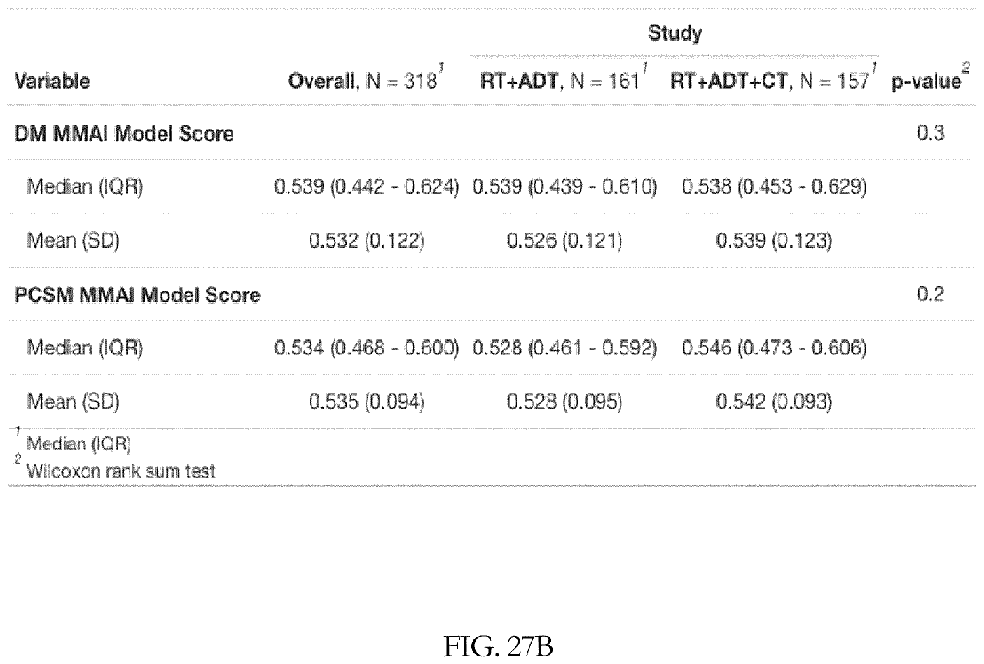

MMAI algorithm scores were generated for 318 of the 397 original clinical trial patients enrolled in NRG/RTOG-9902 (85% of the full trial cohort had available slides; of which 5.6% failed to be included due to poor image quality). FIG. 26 demonstrates the flow of patients from the NRG/RTOG 9902 clinical trial to the DPEP included for model validation. The baseline characteristics of the study DPEP are shown in FIG. 27. The evaluable population included men with median baseline PSA 23.0 ng/mL, 32% of which had cT3-4 disease, 67% had Gleason Grade group 4 or 5 disease, and 54% had >1 NCCN high risk features. At the median follow-up of 10.1 years, 42 men had experienced DM and 29 with PCSM. There are no statistically significant differences in baseline characteristics between patients where MMAI algorithm scores could be obtained compared to the 62 patients that were excluded from this validation study. Similarly, there were no baseline characteristic differences between the two treatment arms within the DPEP. The median (IQR) score of the algorithm optimizing for DM (DM MMAI) was 0.54 (0.44-0.62) and 0.53 (0.47-0.60) for the algorithm optimized for PCSM (PCSM MMAI). Both scores were similar between the 2 treatment arms of NRG/RTOG 9902 (FIG. 27B).

### Model Performance

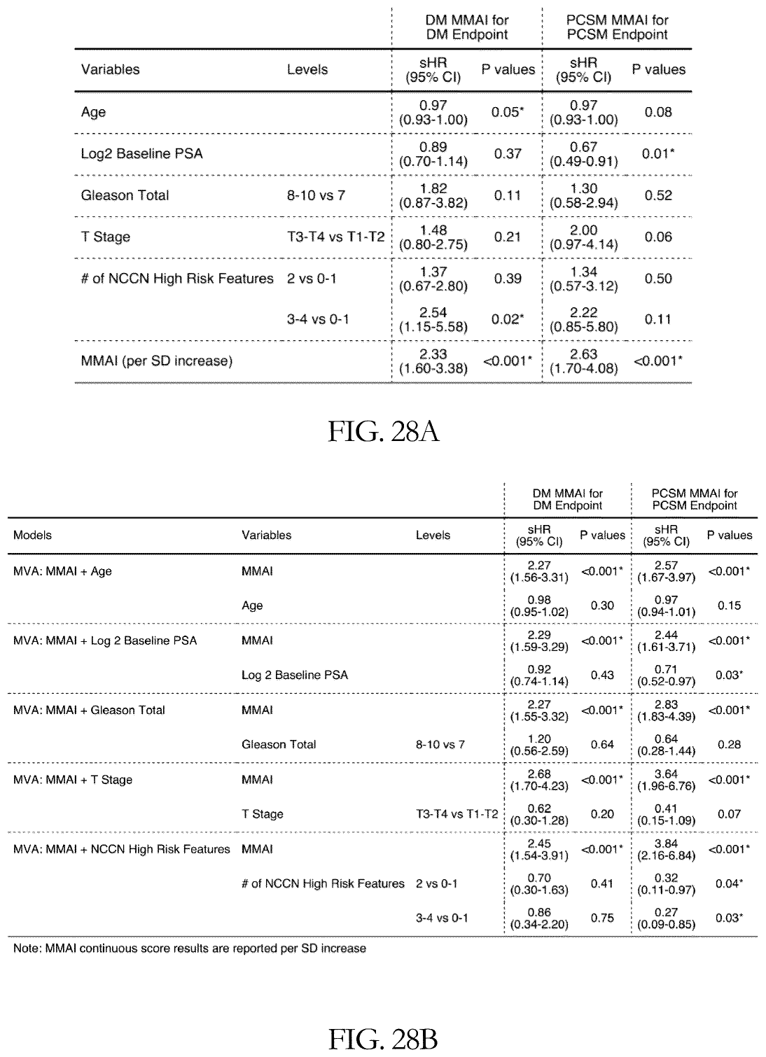

Compared to clinical and pathological factors, the MMAI algorithms were significantly prognostic across outcome measures. In the univariable analysis, the DM MMAI algorithm continuous score was statistically associated with the DM endpoint (sHR 2.33, 95% CI 1.60-3.38, p<0.001), and the PCSM MMAI algorithm for the PCSM endpoint (HR 2.63, 95% CI 1.70-4.08, p<0.001) (FIG. 28A). When evaluating non-optimized secondary endpoints, DM MMAI was statistically significantly associated with risks of BF, PCSM, and OS. Similarly, PCSM MMAI was statistically significantly associated with risks of DM, and OS. (FIG. 28B).

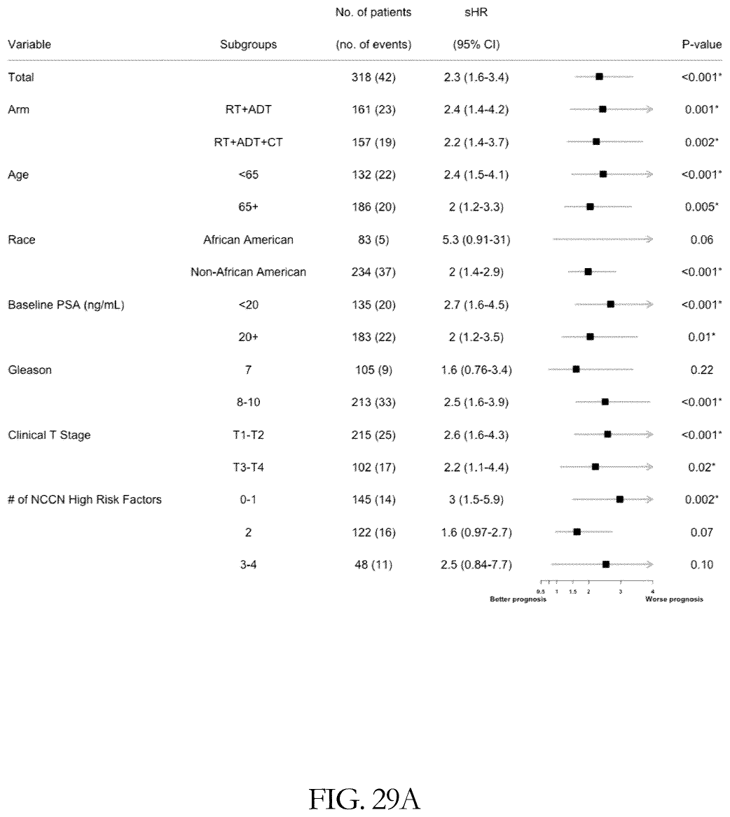

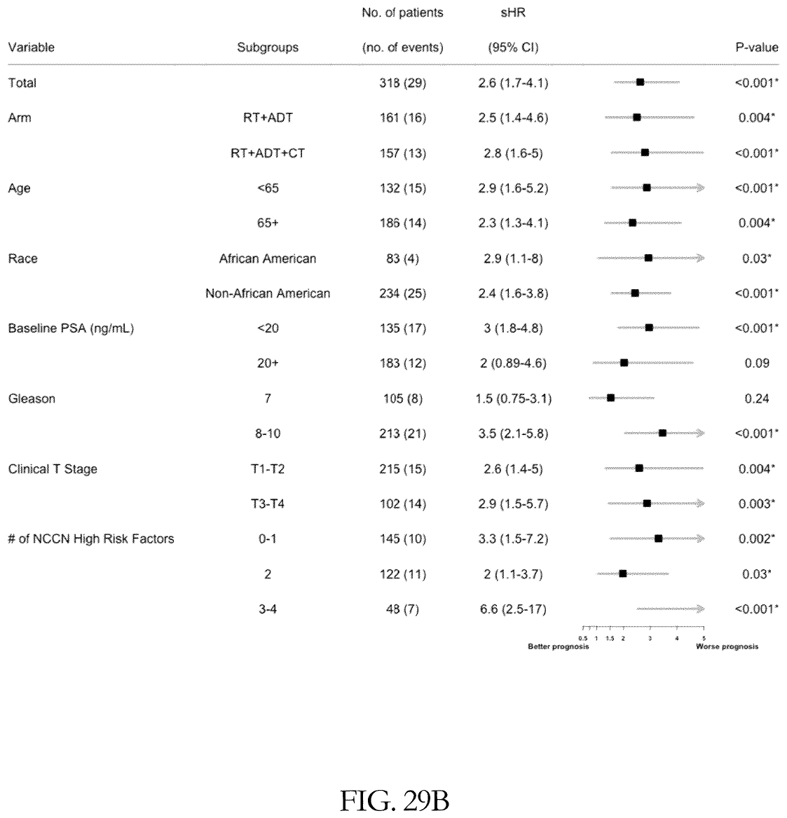

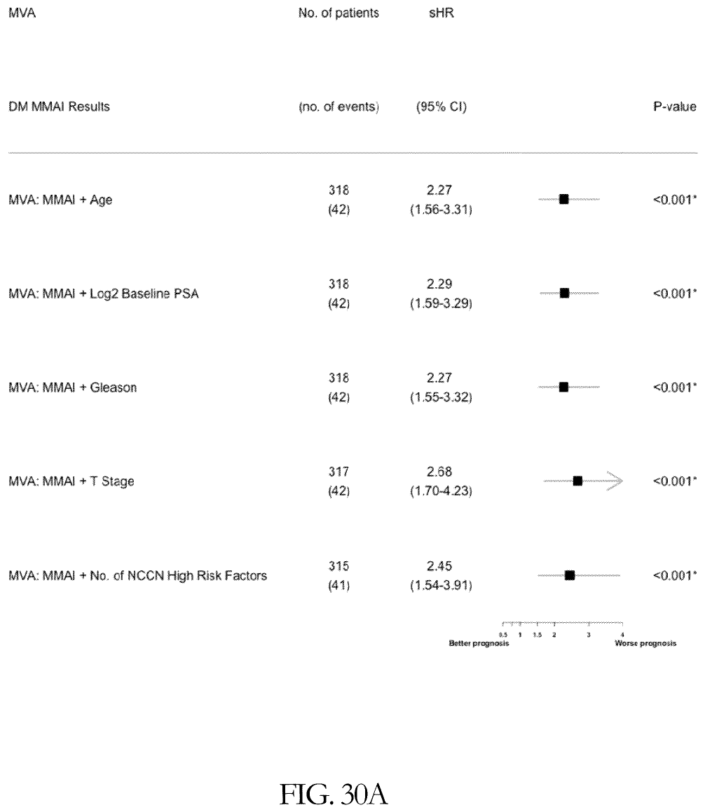

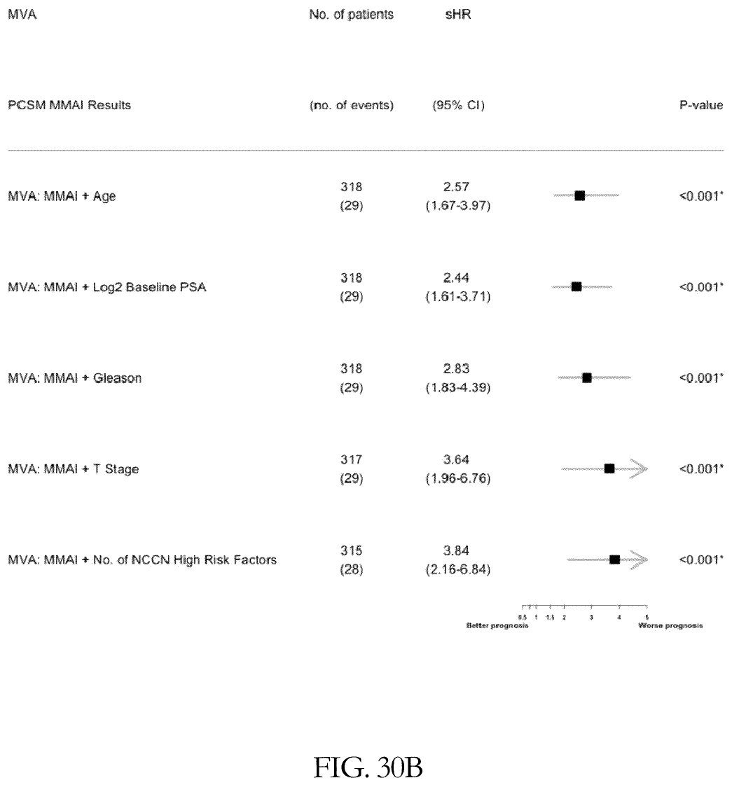

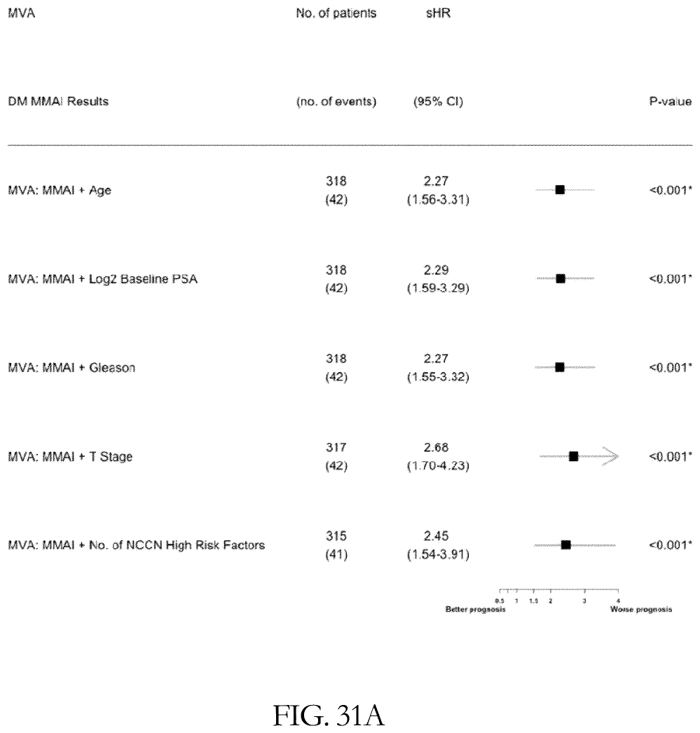

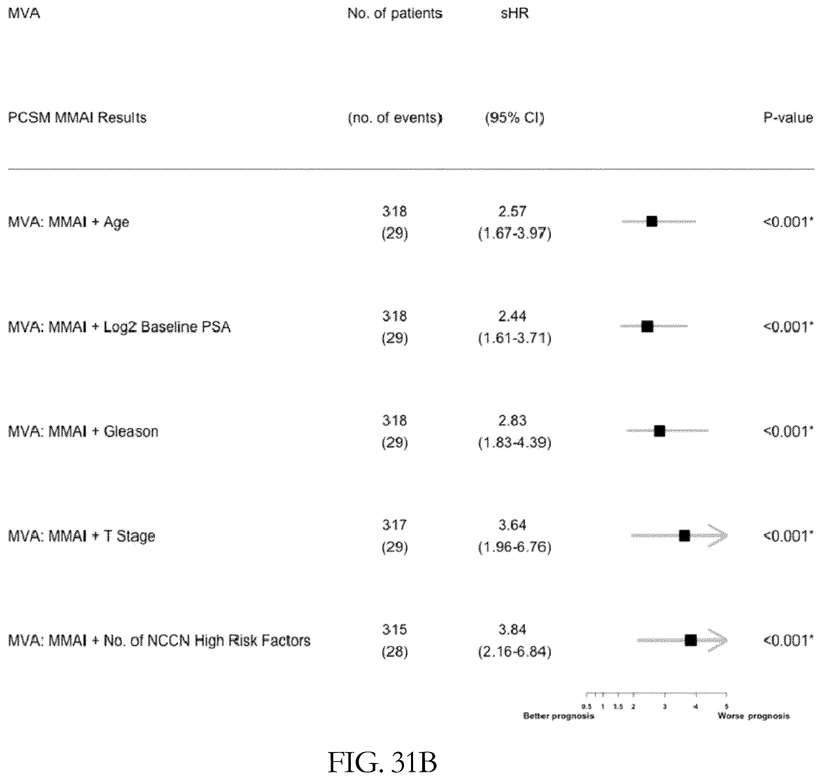

For the DM endpoint, the DM MMAI was prognostic in most clinical subgroups, including both treatment arm, age, non-African American, both PSA group, Gleason 8-10, clinical T-stage, and patients with 1 NCCN high risk factors (FIG. 29A). Similarly, for the PCSM endpoint, the PCSM MMAI was also prognostic in most of the subgroups, including both treatment arms, age, both race subgroups, PSA<20 ng/mL, Gleason 8-10, clinical T-stage, and patients with any NCCN high risk factors (FIG. 29B). In the multivariable analysis (FIGS. 30A-30B and 31A-31B), controlling for individual clinical factors including age, baseline PSA, Gleason, T stage, or number of NCCN high risk factors, both DM MMAI and PCSM MMAI were consistently significant prognostic.

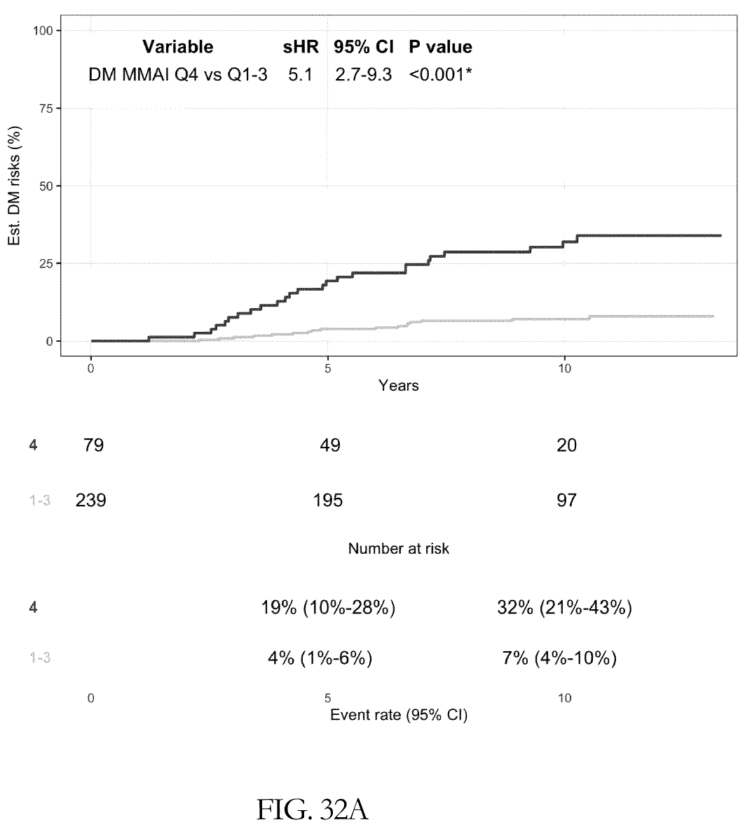

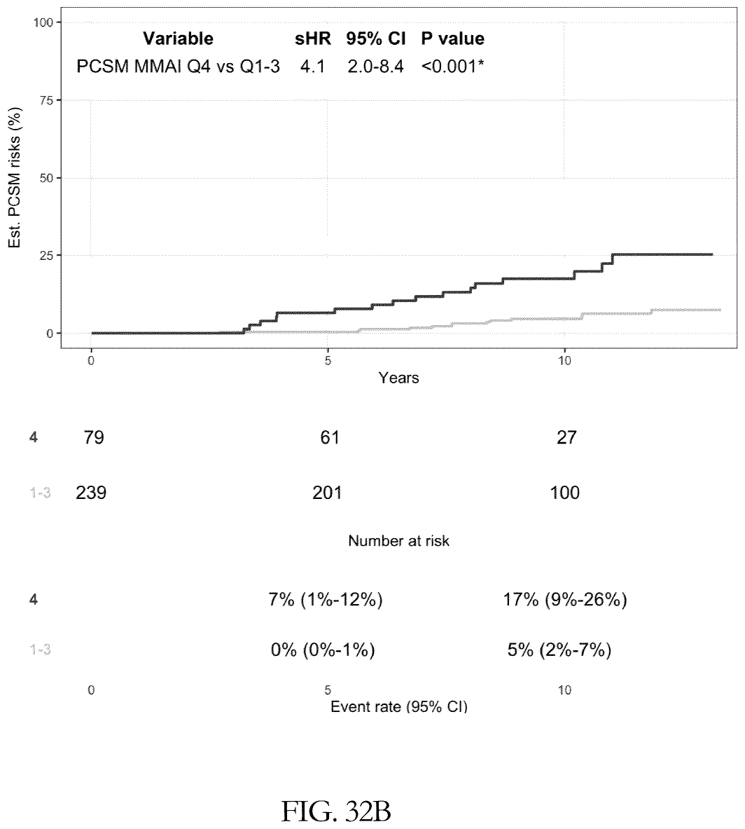

Using quartile splits on the DM MMAI, the lower 75% (Q1-3) of patients had an estimated 5-yr and 10-yr DM rates of 4% (95% CI 1%-6%) and 7% (95% CI 4%-10%), and the highest quartile (Q4) had an estimated 5- and 10-yr DM rates of 19% (95% CI 10%-28%) and 32% (95% CI 21%-43%) with sHR of 5.1 (95% CI 2.7-9.3, p<0.001) (FIG. 32A). Similar results were observed for the PCSM MMAI (sHR 4.1, 95% CI 2.0-8.4, p<0.001) (FIG. 32B).

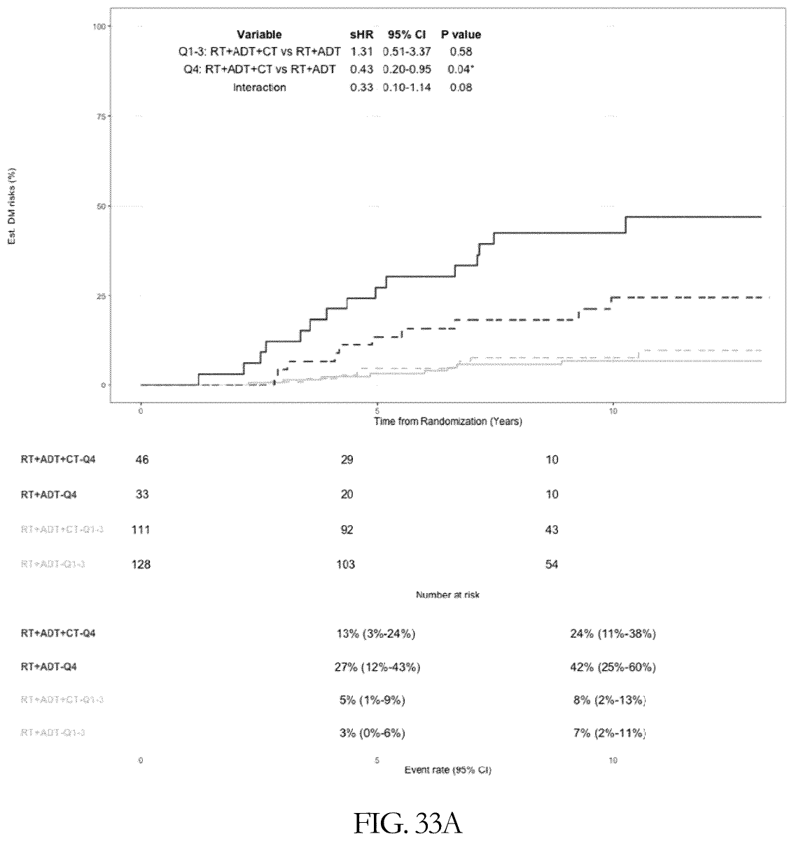

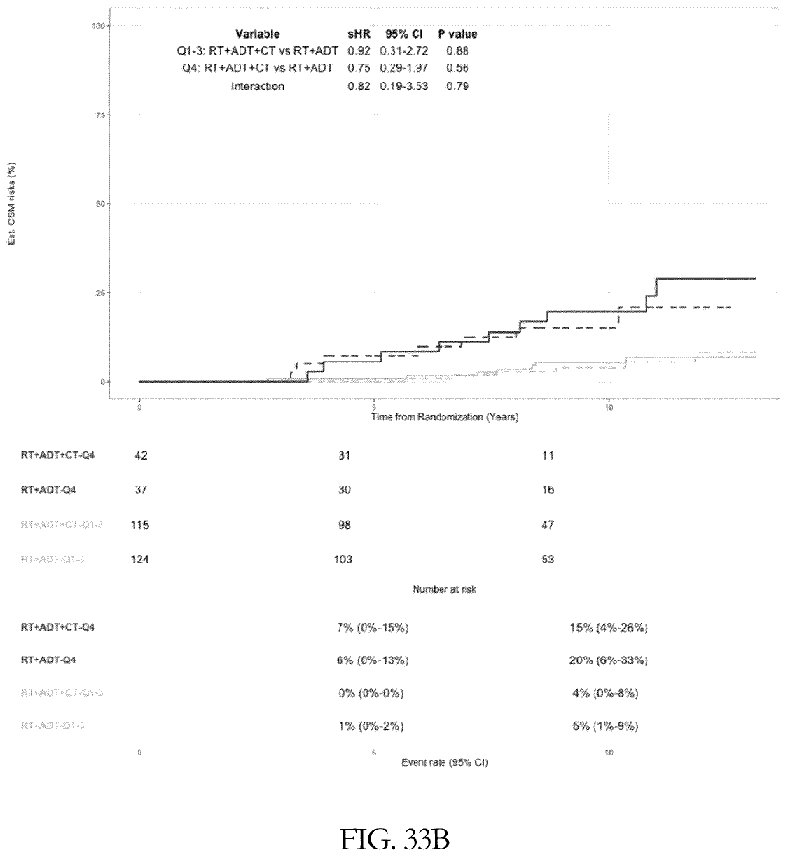

There was no statistically significant interaction between either DM MMAI quartile groups (Q4 vs Q1-3) and CT treatment effect (interaction p=0.08), or PCSM MMAI quartile groups and CT treatment effect (interaction p=0.79) (FIGS. 33A and 33B). Among the top 25% of patients ranked by DM MMAI, the estimated 5-yr absolute benefit from additional use of CT was 14% and 18% for 10-yr (FIG. 33B).

### Discussion

The prognostic ability of MMAI classifiers previously developed using men from five phase III PCa trials (NRG/RTOG 9202, 9408, 9413, 9910, and 0126) was further validated using an external validation set utilized men from NRG/RTOG 9902 which enrolled men who were at high risk for disease progression. The parent trial, NRG/RTOG 9902, did not yield statistically significant clinical results when comparing the treatment arms, so the validation sample was treated as one singular cohort (study arms were well balanced and the mean MMAI classifier score was similar between treatment arms). The differences between MMAI classifier lower risk versus higher risk groups for DM and PCSM were large and statistically significant even among a NCCN high- and very high-risk population. In multivariable analyses, the MMAI score was independently prognostic, even after controlling for variables known to be associated with prognostic risk (patient age, Gleason score, T stage). Association of the MMAI classifiers with DM and PCSM within subgroups suggested added discrimination and prognostic ability of the MMAI throughout the continuum of high- and very high-risk disease.

### Example 5: Nonmetastatic Castration-Resistant Prostate Cancer Prognostication Via Multi-Modal Deep Learning

The addition of apalutamide (APA) to androgen deprivation therapy (ADT) improved metastasis-free survival (MFS) and second progression-free survival (PFS2) among patients with nonmetastatic castration-resistant prostate cancer (nmCRPC). A digital histopathology-based multimodal AI (MMAI) algorithm as described herein was trained to evaluate whether MMAI could define risk of progression among nmCRPC patients treated with APA or placebo in the SPARTAN trial.

Patients enrolled in the SPARTAN trial with available H&E-stained biopsy slides from their primary diagnosis were included. H&E slides were digitized. Baseline clinical parameters to generate MMAI scores were Gleason score, age, T stage, and PSA. MMAI scores for distant metastasis (DM) were generated, ranging from 0 to 1. Patients were further categorized into MMAI non-high-risk and high-risk groups using a score cutoff. Kaplan Meier estimates were calculated for PFS2 and MFS; comparisons were performed using log-rank test and Cox proportional-hazards regression for treatment arms and MMAI risk groups. Two-way ANOVA was used to evaluate the interaction between treatment arms and risk groups.

The study included 471 patients with 1051 biopsy pathology slides: 311 patients treated with APA, and 156 with placebo. 55 patients were excluded due to missing treatment (n=4) or clinical data (n=49) and inadequate H&E images (n=2), resulting in 273 evaluable APA-treated and 147 placebo-treated patients. 63% of patients were MMAI high risk and 37% MMAI non-high risk. MMAI high risk patients demonstrated significant improvement in MFS with APA (HR 0.19 (95% CI: 0.12-0.29, p<0.005)), but not in PFS2 (HR 0.76 (95% CI: 0.45-1.28, p=0.30)). There was a significant interaction between MMAI risk group and treatment for MFS (p=0.02). Among the placebo-treated cohort, MMAI high risk status was associated with shorter MFS (HR 2.98 (95% CI: 1.72-5.18, p<0.005)) and PFS2 (HR 1.83 (95% CI: 1.09-3.09, p=0.02)). For APA-treated patients, MMAI risk group was not associated with MFS and PFS2.

MMAI models as described herein may provide prognostic risk stratification for nmCRPC patients and that MMAI high-risk patients may benefit most from treatment with APA.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### Example 6: Digital Histopathology-Based Multimodal Artificial Intelligence Scores for Predicting Outcomes in a Randomized Phase III Trial in Patients with Nonmetastatic Castration-Resistant Prostate Cancer

Using methods and systems of the present disclosure, digital histopathology-based multimodal artificial intelligence scores were used to predict outcomes in a randomized phase III trial in patients with nonmetastatic castration-resistant prostate cancer.

Androgen deprivation therapy (ADT) is frequently utilized in patients with prostate cancer experiencing serologic recurrence following local therapy. Although ADT therapy is initially effective, nearly all patients develop resistance to ADT, and progress to castration-resistant prostate cancer (CRPC) despite continuous ADT, mainly detected by PSA progression. Men with non-metastatic CRPC (nmCRPC) (defined by PSA progression in the absence of detectable metastases by conventional imaging despite ongoing ADT) are at substantial risk of developing metastases and prostate-cancer-specific death (PCSM). Apalutamide is a standard-of-care androgen receptor inhibitor approved for the treatment of nmCRPC patients, based on the results of the phase III, randomized, placebo-controlled SPARTAN study, which demonstrated that the addition of apalutamide to ongoing ADT significantly improved metastasis-free survival (MFS), time to symptomatic progression, time to second progression (PFS2), and overall survival (OS). Currently, there are no established biomarkers available to guide the personalized use of apalutamide among men with nmCRPC. The development of novel biomarkers may provide useful prognostic and predictive information to aid in clinical decision making.

In this study, the application of MMAI models to nmCRPC using specimens from patients enrolled in the SPARTAN trial was tested. The prognostic utility of MMAI as a risk stratification tool to identify patients with nmCRPC at greater risk of developing distant metastasis (DM) was demonstrated. Finally, the interaction between MMAI risk score and treatment group to demonstrate the utility of MMAI as a predictive marker.

### Study Design

SPARTAN (NCT01946204) was an international, multicenter, randomized, double-blinded, placebo-controlled, phase III clinical trial that evaluated the efficacy of apalutamide in improving MFS in men with nmCRPC. The study enrolled a total of 1207 patients with castration-resistant prostate cancer (defined as three PSA rises, at least 1 week apart, with the last PSA greater than 2 ng/mL demonstrated during continuous ADT). These patients were considered at high risk for the development of metastases, as indicated by a PSA doubling time of 10 months or less during continuous ADT.

Participants were randomly assigned 2:1 to receive ongoing ADT with either apalutamide (240 mg daily) or a matched placebo. A primary endpoint of the study was MFS, defined as the duration from randomization to the first detection of distant metastasis on conventional imaging (as determined on central review by independent radiologists blinded to patient identifiers and treatment) or death from any cause, whichever occurred first. Secondary endpoints included second progression-free survival (PFS2), defined as the time from randomization to progression on first subsequent therapy, and overall survival (OS), defined as the time from randomization to death.

With a median follow up of 52 months, SPARTAN demonstrated that the addition of apalutamide to ongoing ADT led to a significant extension of OS compared to placebo. Analysis of genomic classifier (GC) biomarker for patients treated in the SPARTAN trial did not find a statistically significant interaction between GC score and treatment group.

### Specimens and MMAI Score Generation

MMAI prostate models were developed and validated based on five multinational prospective phase III randomized trials (NRG/RTOG 9202, 9408, 9413, 9910, and 0126). These models exhibited superior prognostic performance compared to the standard clinical risk models (NCCN) in predicting distant metastasis (DM) and prostate cancer-specific mortality (PCSM). The patients included in these trials were randomized to various combinations of external radiotherapy with or without ADT.

The MMAI models were developed using digitized histopathology slides of core prostate biopsies, along with clinical factors such as Gleason total score, Gleason primary and secondary patterns, tumor stage, baseline prostate-specific antigen level (PSA), and age. The training process involved utilizing 80% of the data available from the five clinical trials, while the remaining 20% of trial data was used for validation. A locked version of the MMAI model (Version 1.1) was used to generate MMAI scores in the SPARTAN trial to evaluate its performance among nmCRPC patients treated with apalutamide or placebo. The score generation process was blinded from the clinical endpoint information.

MMAI scores were computed for each subject within the analysis cohort using whole slide images digitized at 20× resolution from H&E-stained prostate primary slides obtained at the time of diagnosis. These scores, ranging from 0 to 1, represent a continuum of increasing probabilities for clinical events such as DM and PCSM. Subjects' MMAI scores for risk of DM categorized patients into MMAI risk groups (low, intermediate and high) using predefined clinical thresholds established for patients with localized prostate cancer based upon clinician input. For the purpose of this analysis, patients were categorized as either MMAI high risk or MMAI non-high risk (MMAI score with >10% estimated 10-year DM rates defined high-risk), with the latter group combining the low and intermediate risk categories for comparison.

### Statistical Analysis

Summary statistics of baseline characteristics for the analysis cohort, treatment subgroups and the overall clinical trial cohort were reported to evaluate comparability. Kaplan-Meier estimates were calculated for MFS, PFS2, and OS for analysis by treatment group and MMAI risk groups. Comparisons were performed using log-rank test and Cox proportional hazards regression models for treatment arms, MMAI risk scores, and MMAI risk group. The reporting unit for continuous MMAI scores in the Cox models is standard deviation (SD), meaning that each 1-unit increase corresponds to 1 SD increase in MMAI score. The interaction between treatment arm and MMAI risk scores was evaluated using a Cox proportional hazards model. Treatment effects within each MMAI risk subgroup were also assessed to measure the relative treatment effect between arms.

### Cohort Selection and Patient Characteristics

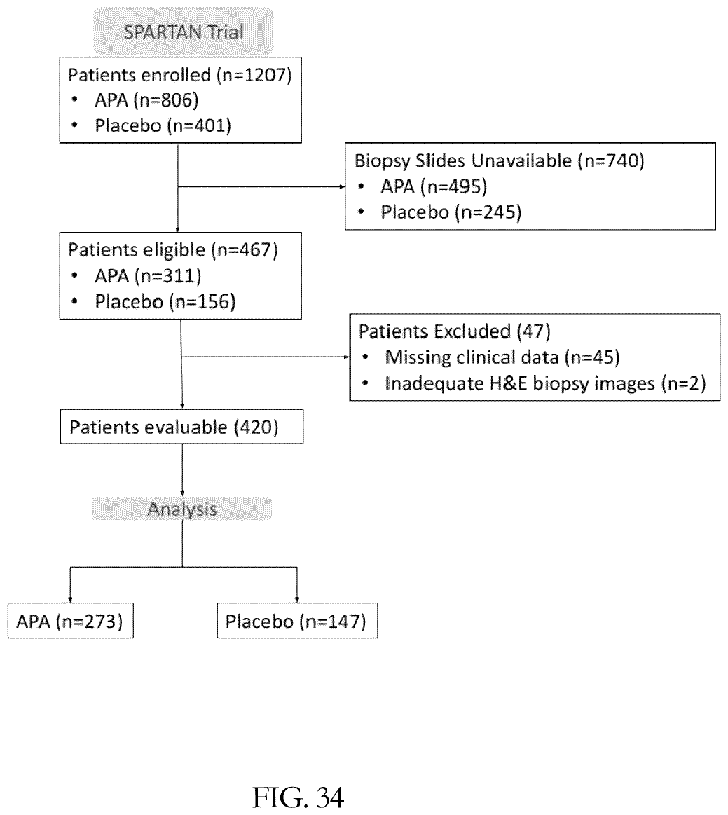

Of the 1,207 patients enrolled in SPARTAN, a total of 467 patients with 1051 pathology slides were included in this study, of which 311 were treated with apalutamide and 156 received placebo. After excluding patients with missing clinical data (n=45) and/or inadequate H&E images (n=2), a final analysis cohort of 420 evaluable patients remained, comprising 273 apalutamide-treated patients and 147 placebo-treated patients. See FIG. 34 for a cohort selection diagram. Baseline characteristics were comparable for the analysis cohort compared to the overall trial population, as well as between the treatment arms (Table 1). Among the evaluated 420 patients, 63% (n=266) were classified as MMAI high risk, while 37% (n=154) were categorized as MMAI non-high risk.

TABLE 1
Baseline patient characteristics.

| | | Overall SPARTAN Trial | | | | MMAI Biomarker Cohort | | | |
|---|---|---|---|---|---|---|---|---|---|
| | | Apalutamide + | | Placebo + | | Apalutamide + | | Placebo + | |
| | | ADT | | ADT | | ADT | | ADT | |
| | | N = 806 | | N = 401 | | N = 311 | | N=156 | |
| | | (66.8%) | | (33.2%) | | (66.6%) | | (33.4%) | |
| Age, median (range) years | | 74.0 | (48, 94) | 74.0 | (52, 97) | 74.0 | (49, 91) | 73.5 | (52, 90) |
| PSA (ng/ml), mean (SD) | | 14.9 | (22.53) | 15.9 | (23.75) | 15.9 | (23.6) | 16.6 | (19.6) |
| Tumor stage, N (%) | T1 | 141 | (17.8) | 63 | (16.0) | 52 | (16.9) | 27 | (17.8) |
| | T2 | 265 | (33.4) | 123 | (31.2) | 110 | (35.8) | 43 | (28.3) |
| | T3 | 296 | (37.3) | 163 | (41.4) | 107 | (34.9) | 69 | (45.4) |
| | T4 | 32 | (4.0) | 16 | (4.1) | 12 | (3.9) | 8 | (5.3) |
| | Tx | 60 | (7.6) | 29 | (7.4) | 26 | (8.5) | 5 | (3.3) |
| Gleason score, N (%) | <7 | 152 | (19.4) | 72 | (18.6) | 37 | (12.1) | 27 | (17.3) |
| | =7 | 291 | (37.1) | 146 | (37.7) | 124 | (40.5) | 64 | (41.0) |
| | >7 | 341 | (43.5) | 169 | (43.7) | 145 | (47.4) | 65 | (41.7) |
| Local or regional | N0 | 673 | (83.5) | 336 | (83.8) | 249 | (80.1) | 126 | (80.8) |
| nodal disease, N (%) | N1 | 133 | (16.5) | 65 | (16.2) | 62 | (19.9) | 30 | (19.2) |

| Median metastasis- | 40.5 | (NR, NR) | 16.2 | (14.6, 18.4) | NR | (29.1, NR) | 14.6 | (12.5, 19.0) |
|---|---|---|---|---|---|---|---|---|
| free survival | | | | | | | | |
| (95% CI), month | | | | | | | | |
| Median overall | 73.9 | (61.2, NR) | 59.9 | (52.8, NR) | 65.1 | (59.8, NR) | 58.6 | (49.6, NR) |

survival (95% CI), month

ADT, Androgen deprivation therapy;
ng/mL, nanograms per milliliter;
N, number of patients;
SD, standard deviation;
MMAI, Multimodal Artificial Intelligence;
NR: not reached.
% may not add up to 100% due to rounding.

### Clinical Outcomes by MMAI Risk

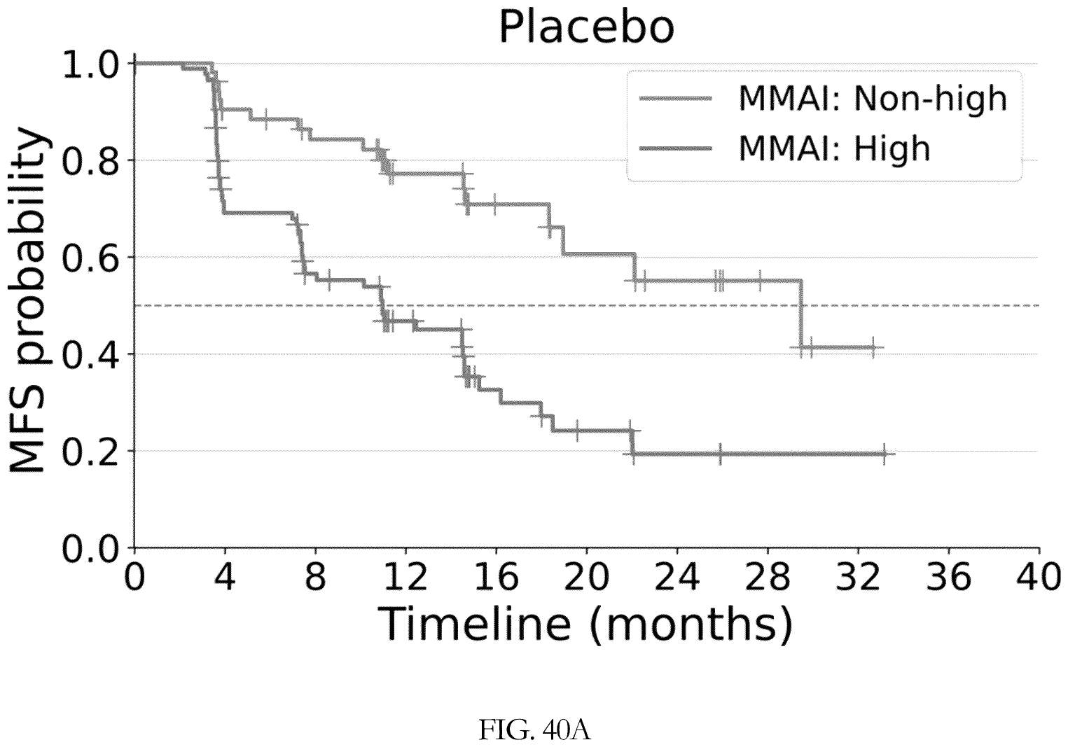

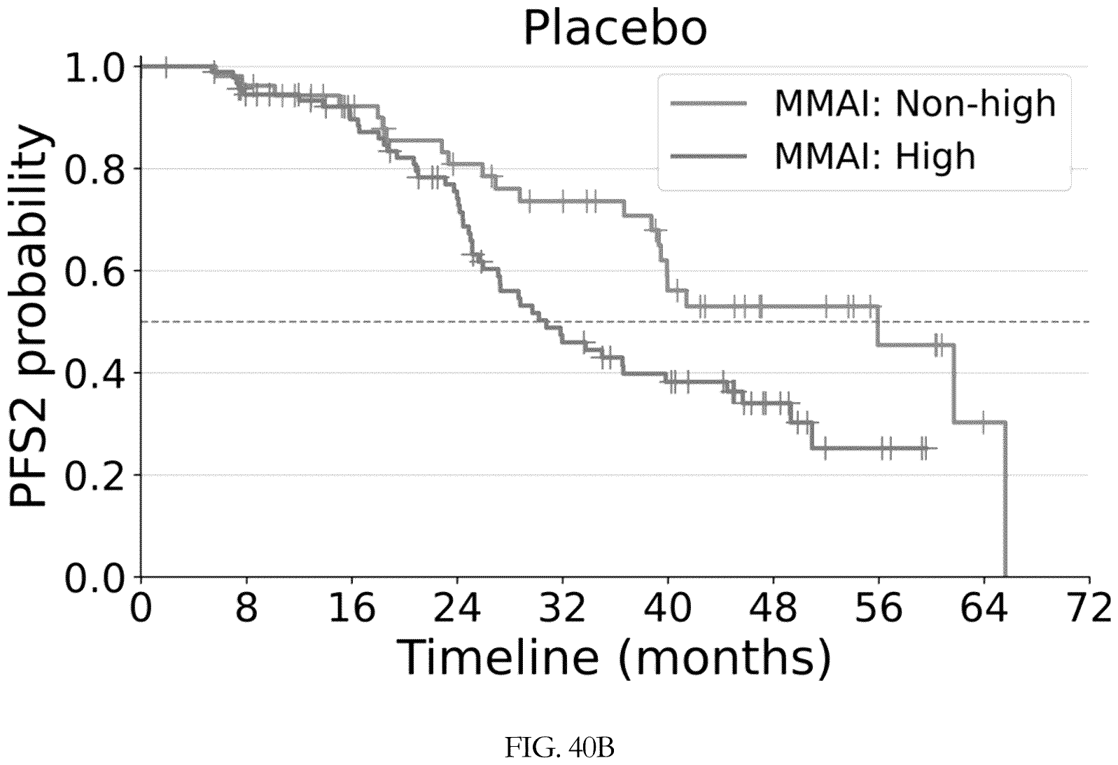

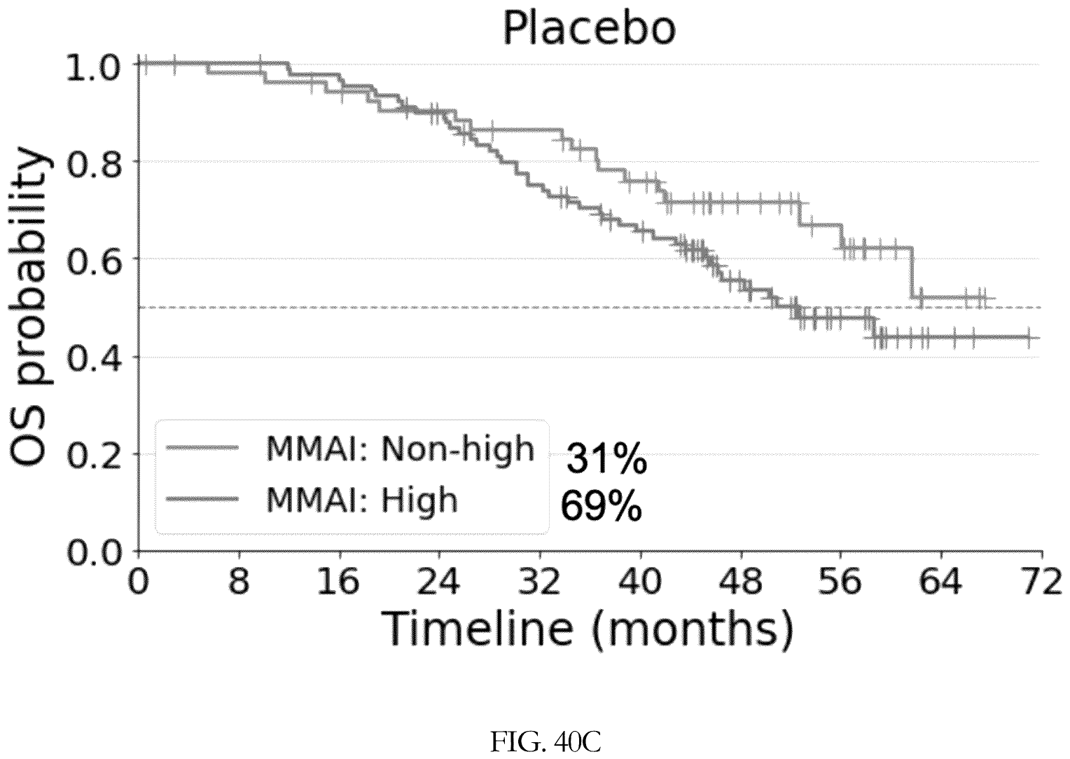

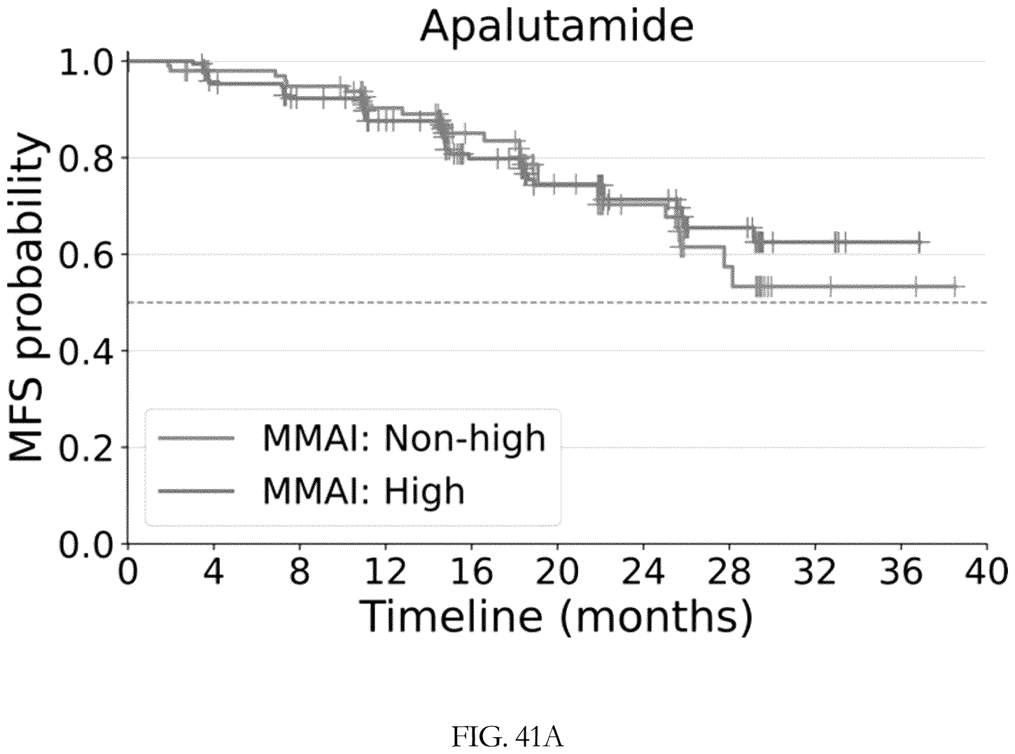

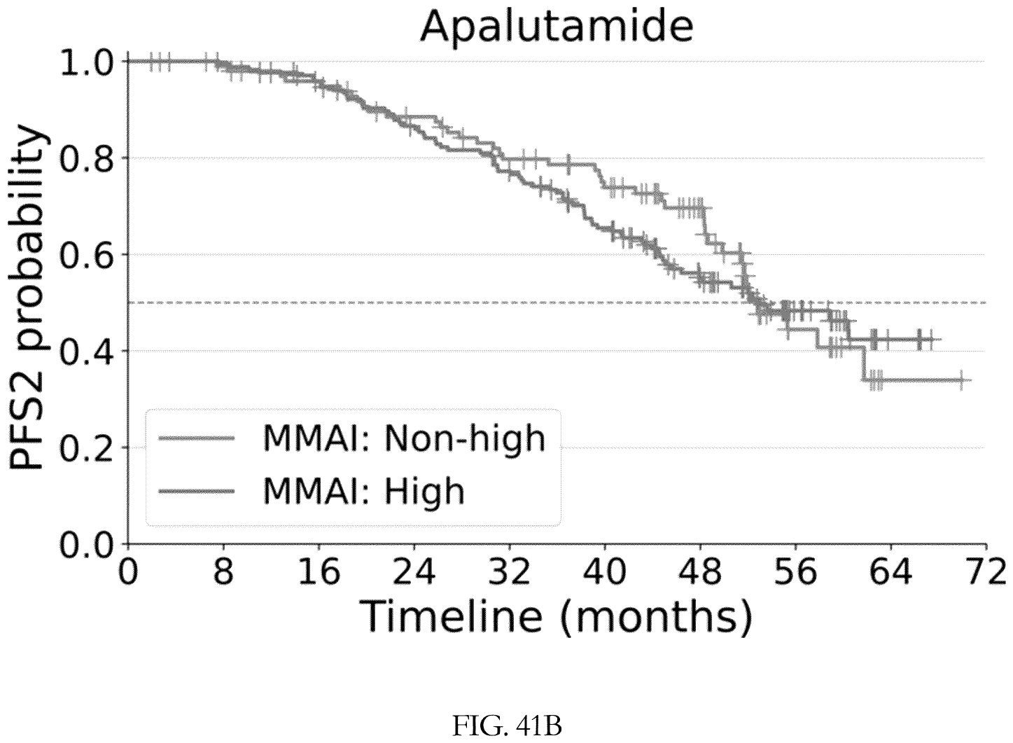

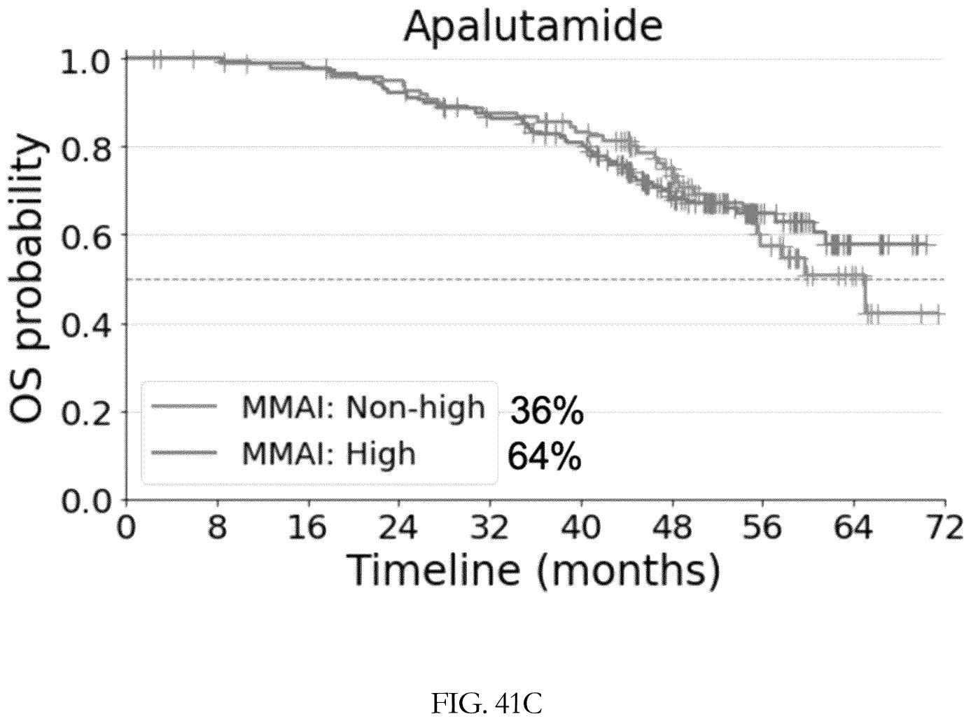

The Cox regressions with MMAI as a continuous variable are shown in Table 2; MMAI risk score was associated with shorter MFS (hazard ratio [HR]1.72, 95% CI: 1.34-2.21, p<0.005), PFS2 (HR 1.57, 95% CI: 1.20-2.05, p<0.005), and OS (HR 1.41, 95% CI 1.06-1.87, p=0.02). In the Cox regression analysis of the overall analysis cohort, MMAI high risk group had a hazard ratio (HR) of 1.47 (95% CI: 1.03-2.11, p=0.04) compared to MMAI non-high risk patients for MFS and had a HR of 1.30 (95% CI: 0.96-1.76, p=0.09) for PFS2 and a HR of 1.15 (95% CI: 0.82-1.61, p=0.42) for OS (FIGS. 40A-40C). Within the placebo-treated cohort, MMAI high risk status was associated with a shorter MFS, with an HR of 2.98 (95% CI: 1.72-5.18, p<0.005), as well as a shorter PFS2, with an HR of 1.83 (95% CI: 1.09-3.09, p=0.02), and OS with an HR of 1.56 (95% CI: 0.89-2.75, p=0.12) (FIGS. 41A-41C).

TABLE 2
Cox regression results for all patients, using continuous MMAI risk score.
MFS, metastasis-free survival; PFS2, progression-free survival; OS, overall survival;
MMAI, Multimodal Artificial Intelligence.
Univariate analysis

| | MFS | | | PFS2 | | | OS | | |
|---|---|---|---|---|---|---|---|---|---|
| Variable | HR | 95% CI | P value | HR | 95% CI | P value | HR | 95% CI | P value |
| MMAI risk score | 1.24 | 1.04-1.47 | 0.01 | 1.20 | 1.03-1.39 | 0.02 | 1.19 | 1.01-1.41 | 0.04 |
| Treatment | 0.28 | 0.20-0.40 | <0.005 | 0.55 | 0.41-0.74 | <0.005 | 0.7 | 0.50-0.97 | 0.03 |

Multivariable analysis

| | MFS | | | PFS2 | | | OS | | |
|---|---|---|---|---|---|---|---|---|---|
| Variable | HR | 95% CI | P value | HR | 95% CI | P value | HR | 95% CI | P value |
| MMAI risk score | 1.72 | 1.34-2.21 | <0.005 | 1.57 | 1.20-2.05 | <0.005 | 1.41 | 1.06-1.87 | 0.02 |
| Treatment | 1.72 | 0.39-7.58 | 0.47 | 2.28 | 0.57-9.03 | 0.24 | 1.98 | 0.44-8.99 | 0.37 |
| MMAI risk score:Treatment | 0.63 | 0.45-0.89 | 0.01 | 0.70 | 0.51-0.97 | 0.03 | 0.78 | 0.55-1.10 | 0.18 |

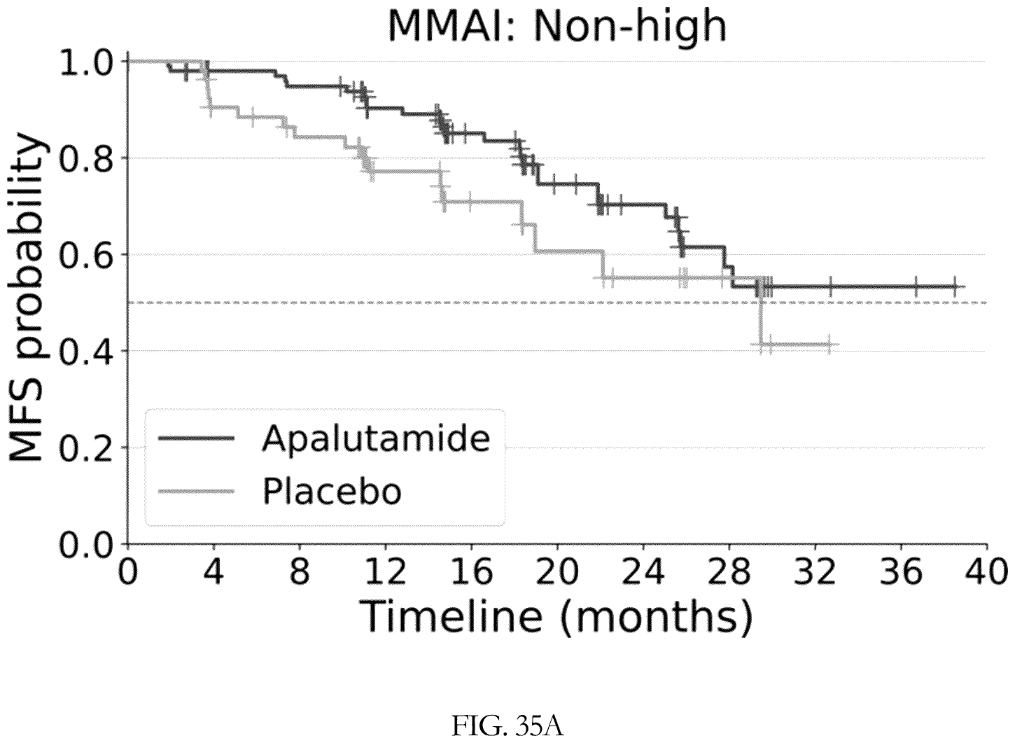

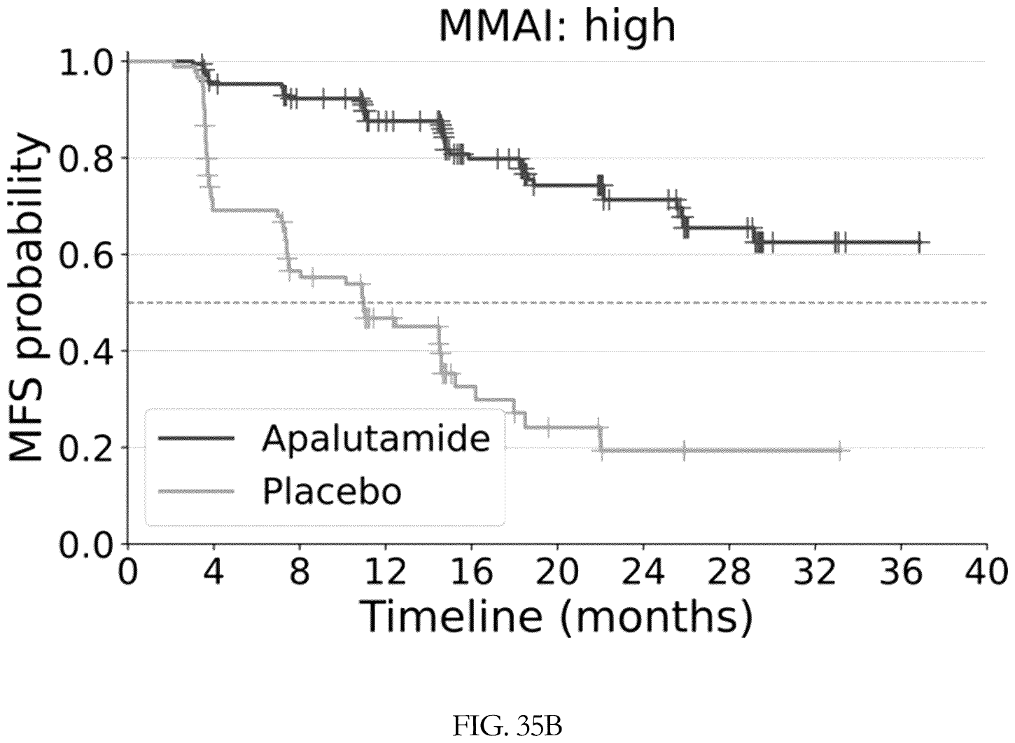

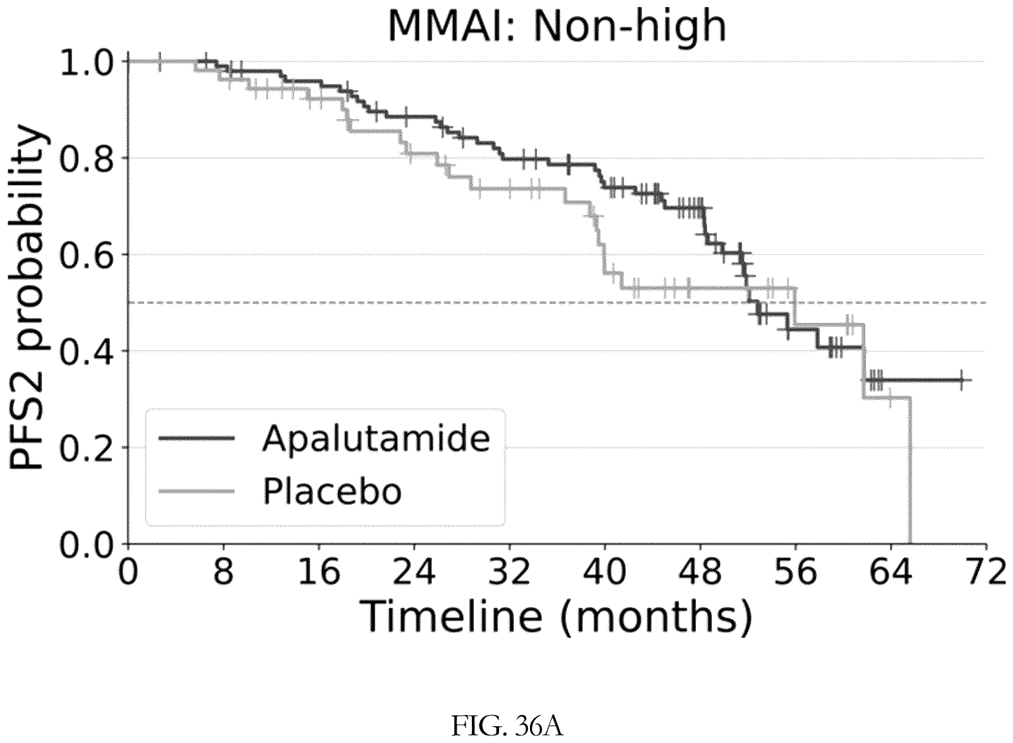

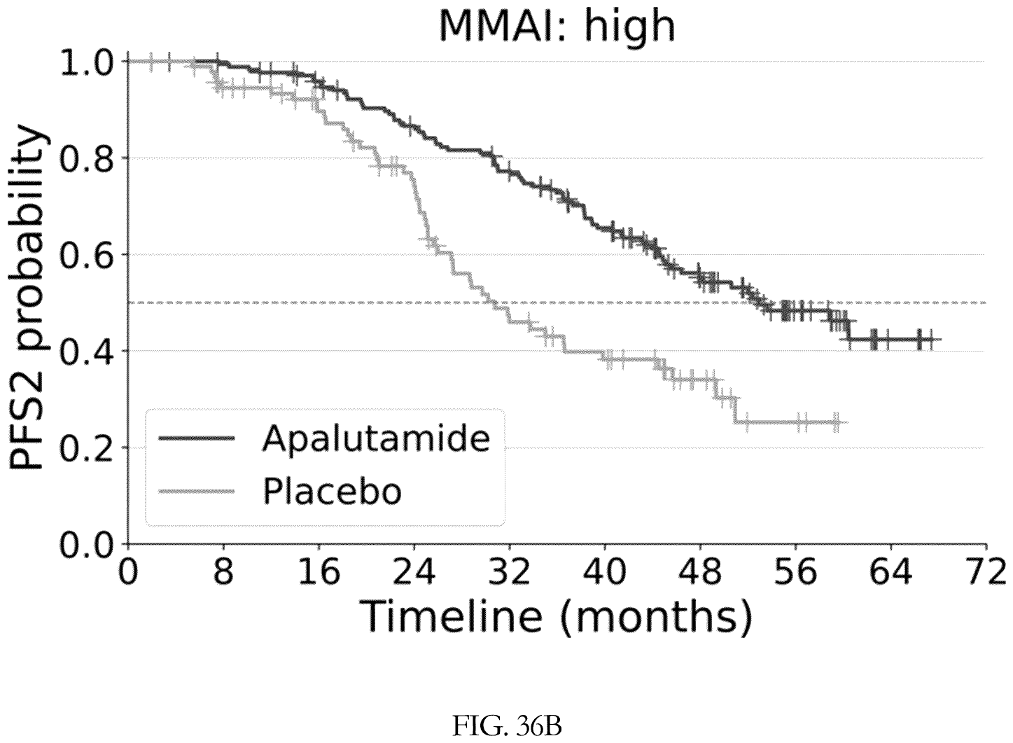

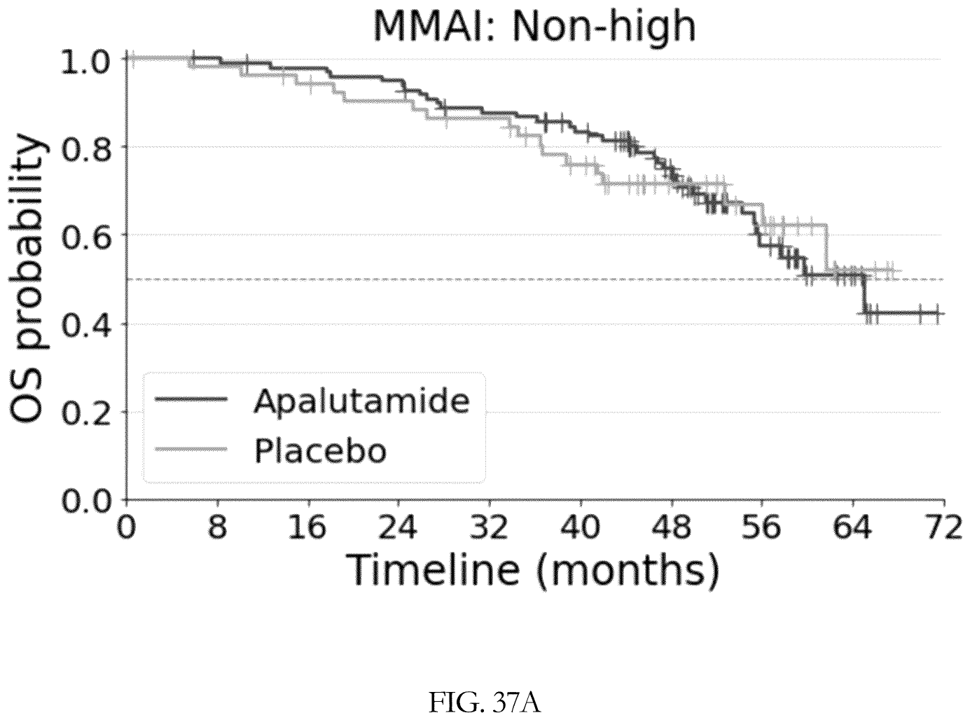

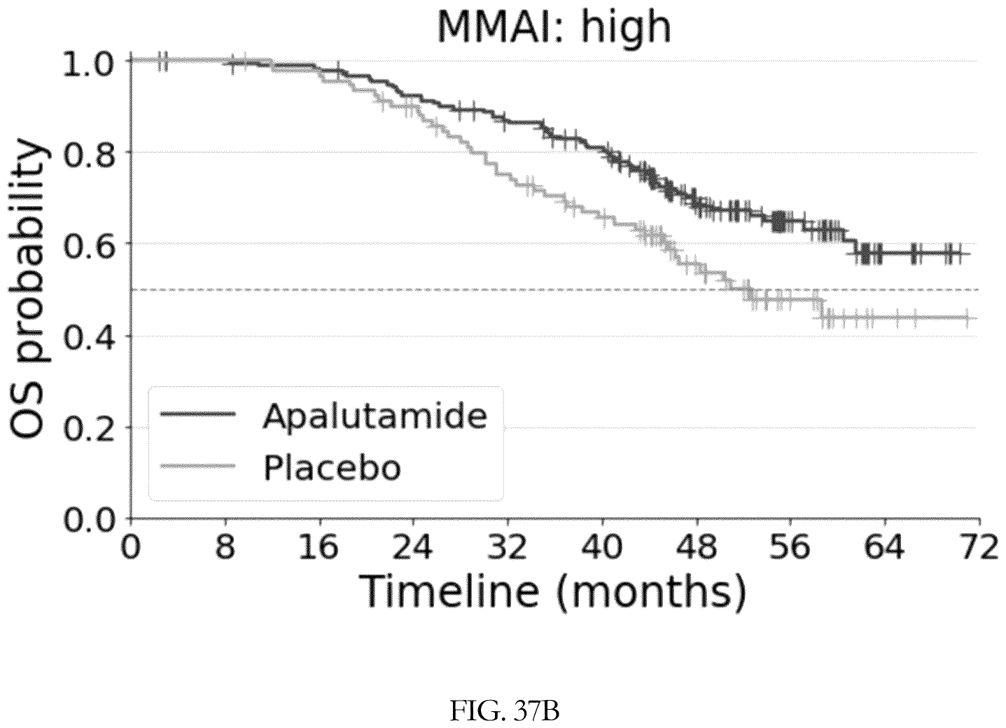

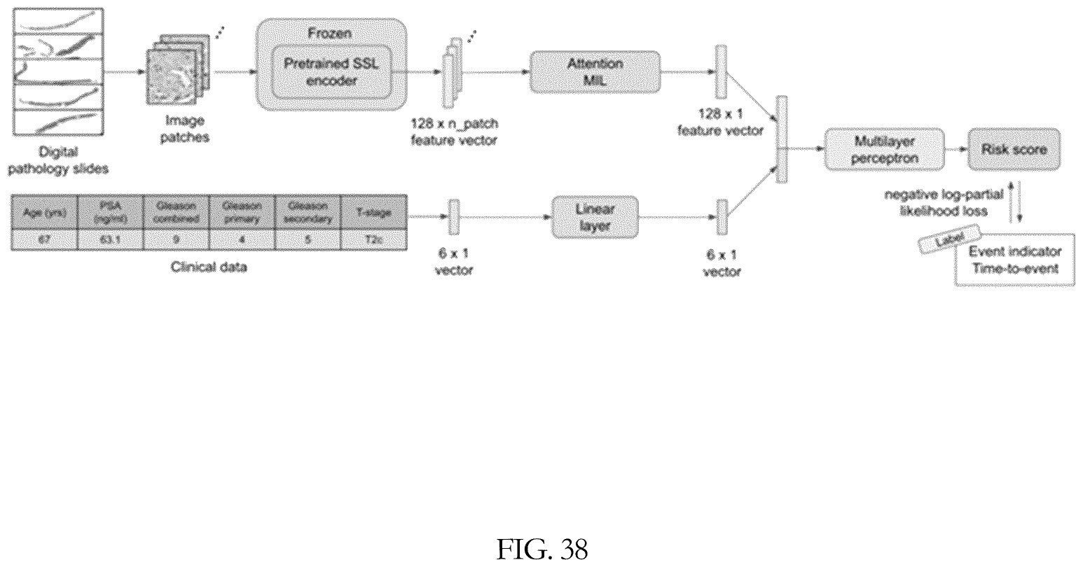

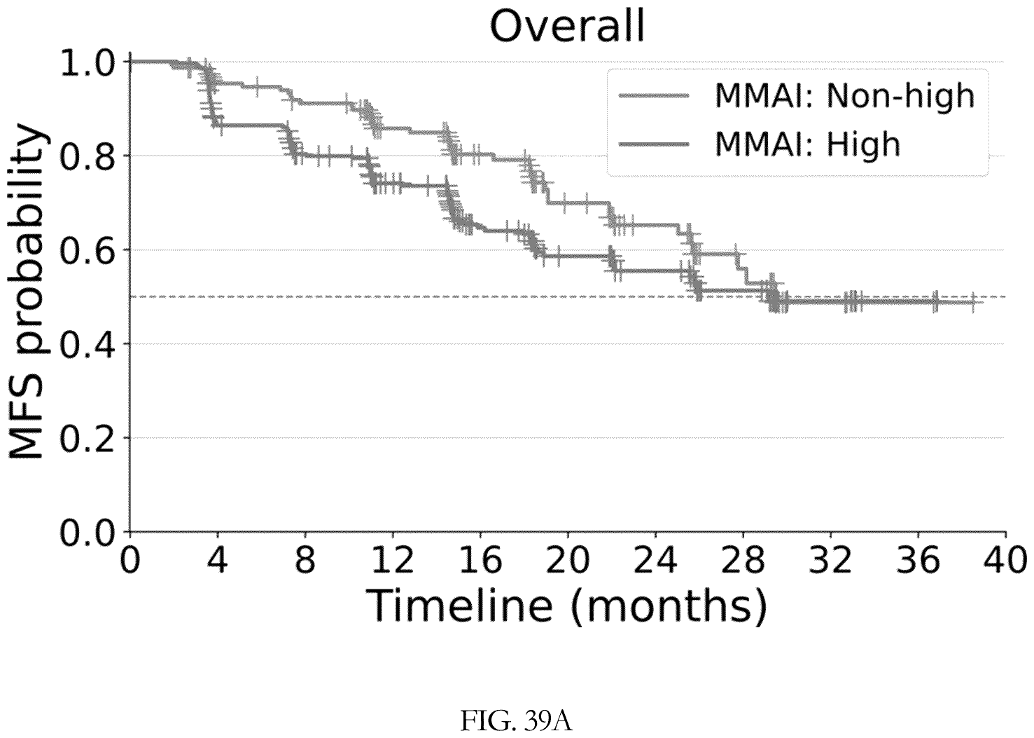

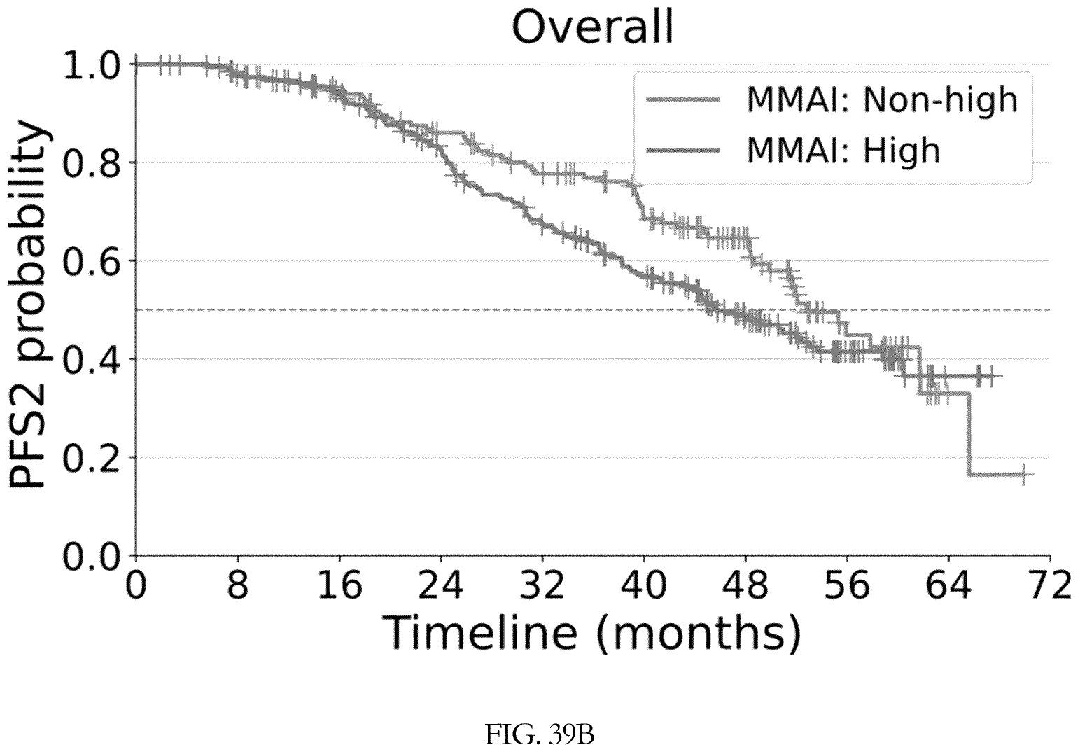

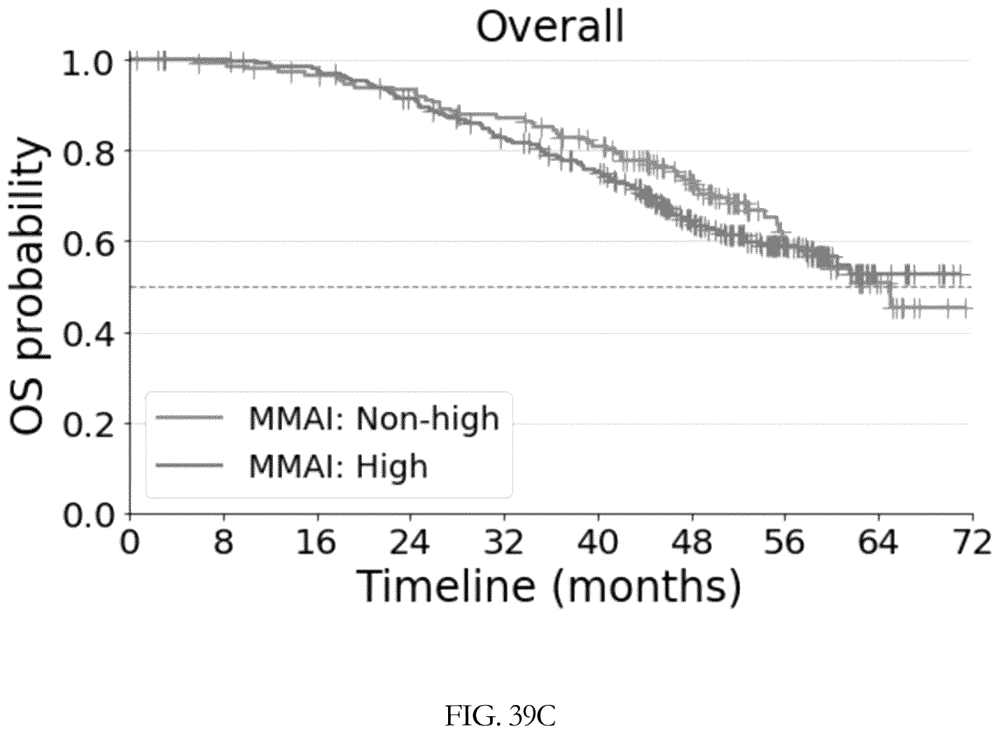

In the MMAI non-high risk group, there was no significant difference observed between apalutamide and placebo for MFS (FIG. 35A), PFS2 (FIG. 36A), and OS (FIG. 37A). In the MMAI high risk group, apalutamide treatment demonstrated a significant improvement compared to placebo in MFS with a HR of 0.19 (95% CI: 0.12-0.29, p<0.005) (FIG. 35B), PFS2 with HR of 0.47 (95% CI: 0.33-0.68, p<0.005) (FIG. 36B), and OS with HR: 0.6, 95% CI: 0.40-0.89, p=0.01 (FIG. 37B).

### Interaction Between MMAI Risk Group and Treatment

A significant interaction was found between the continuous MMAI risk scores and treatment for both MFS (p=0.01) and PFS2 (p=0.03) but not OS (p=0.16) (Table 2), with greater benefit from apalutamide treatment noted in the MMAI high risk group compared to the MMAI non-high risk group. Among apalutamide-treated patients, the MMAI risk group was not associated with significant differences in MFS (p=0.75), PFS2 (p=0.53), or OS (p=0.85) outcomes (FIG. 42). The Cox regressions and interactions with treatment with MMAI risk group are shown in Table 3.

TABLE 3
Cox regression results for all patients, using MMAI risk group.
MFS, metastasis-free survival; PFS2, progression-free survival; OS, overall survival;
MMAI, Multimodal Artificial Intelligence.
Univariate analysis

| | MFS | | | PFS2 | | | OS | | |
|---|---|---|---|---|---|---|---|---|---|
| Variable | HR | 95% CI | P value | HR | 95% CI | P value | HR | 95% CI | P value |
| MMAI risk group (high/non- | 1.47 | 1.03-2.11 | 0.04 | 1.30 | 0.96-1.76 | 0.09 | 1.15 | 0.82-1.61 | 0.42 |
| high) | | | | | | | | | |
| Treatment | 0.28 | 0.20-0.40 | <0.005 | 0.55 | 0.41-0.74 | <0.005 | 0.70 | 0.50-0.97 | 0.03 |

Multivariable analysis

| | MFS | | | PFS2 | | | OS | | |
|---|---|---|---|---|---|---|---|---|---|
| Variable | HR | 95% CI | P value | HR | 95% CI | P value | HR | 95% CI | P value |
| MMAI risk group (high/non- | 3.17 | 1.83-5.48 | <0.005 | 1.83 | 1.11-3.04 | 0.02 | 1.58 | 0.90-2.78 | 0.11 |
| high) | | | | | | | | | |
| Treatment | 0.61 | 0.33-1.13 | 0.11 | 0.74 | 0.44-1.25 | 0.26 | 0.97 | 0.55-1.74 | 0.93 |
| MMAI risk group:Treatment | 0.29 | 0.14-0.61 | <0.005 | 0.61 | 0.33-1.16 | 0.13 | 0.60 | 0.30-1.22 | 0.16 |

In this study, MMAI risk score was shown to be an important prognostic marker in nmCRPC patients, with higher MMAI risk scores associated with worse MFS, PFS2, and OS. In addition, the MMAI risk score may be an important predictive marker. We demonstrated a significant interaction between MMAI risk and treatment group for both MFS and PFS2 endpoints. The MMAI high risk group demonstrated a greater benefit from the addition of apalutamide compared to the MMAI low risk group in both MFS and PFS2. While all patients with nmCRPC were shown to benefit from the addition of apalutamide to ADT in the SPARTAN trial, these findings demonstrate that MMAI high-risk patients may derive the most benefit.

These findings present the first validation of a digital histopathology-based MMAI model using information obtained from the prostate primary tumor in the hormone-naïve setting, for prognostic risk stratification later in the course of the disease, when patients had developed castration resistant prostate cancer, in the nmCRPC setting. Furthermore, these findings demonstrate that in addition to serving as a prognostic marker, the digital histopathology-based MMAI model may also serve as a predictive marker for nmCRPC patients treated with apalutamide.

These findings provide important insights into the clinical utility of the MMAI model in a distinct clinical context well beyond localized prostate cancer, when patients have developed castration resistant prostate cancer, at time of primary management decisions.

The findings of this study have several clinical implications. First, MMAI can effectively risk-stratify nmCRPC patients to help inform treatment decisions and aid clinical trial eligibility. Further, MMAI risk groups may be used to identify patients who are more likely to benefit from apalutamide, or potentially other forms of androgen receptor-targeted treatment intensification, to aid in personalized therapy selection. Apalutamide, an androgen receptor inhibitor, may show significant benefits in terms of MFS, time to symptomatic progression, PFS2, and OS in patients with nmCRPC. However, not all patients may derive equal benefit from the addition of apalutamide therapy. The MMAI algorithm may help identify those patients who are at higher risk of progression and would benefit the most from apalutamide, thus optimizing treatment selection and potentially improving patient outcomes.

Currently, there are no established biomarkers available to guide the individualized use of apalutamide in this patient population. Blood biomarkers and a tissue-based genomic classifier may help identify patients who may benefit from treatment with apalutamide. These results demonstrate a significant interaction between MMAI risk group and treatment provides strong evidence that integration of the MMAI test into clinical practice may improve treatment decision-making and optimize patient outcomes for nmCRPC. Molecular biomarkers may offer clinical utility when they are likely to affect a clinical decision, thereby making predictive biomarkers particularly compelling for clinical use in identifying patients who benefit most from treatment intensification with novel therapies.

These results demonstrate that the MMAI algorithm can risk-stratify patients with nmCRPC and may help identify those who may benefit most from apalutamide. This digital histopathology-based approach may serve as a valuable tool in personalized medicine for nmCRPC.

### REFERENCES

1. Dai, C., Heemers, H. & Sharifi, N. Androgen Signaling in Prostate Cancer. Cold Spring Harb. Perspect. Med. 7, a030452 (2017) is incorporated by reference herein in its entirety.
2. NCCN. NCCN Clinical Practice Guidelines in Oncology Prostate Cancer. NCCN Guidel. v4.2023, (2023) is incorporated by reference herein in its entirety.
3. Smith, M. R. et al. Apalutamide Treatment and Metastasis-free Survival in Prostate Cancer. N. Engl. J. Med. 378, 1408-1418 (2018) is incorporated by reference herein in its entirety.
4. Smith, M. R. et al. Apalutamide and Overall Survival in Prostate Cancer. Eur. Urol. 79, 150-158 (2021) is incorporated by reference herein in its entirety.
5. Ballman, K. V. Biomarker: Predictive or Prognostic?J. Clin. Oncol. Off. J. Am. Soc. Clin. Oncol. 33, 3968-3971 (2015) is incorporated by reference herein in its entirety.
6. Esteva, A. et al. Prostate cancer therapy personalization via multi-modal deep learning on randomized phase III clinical trials. NPJ Digit. Med. 5, 71 (2022) is incorporated by reference herein in its entirety.
7. Spratt, D. E. et al. Artificial Intelligence Predictive Model for Hormone Therapy Use in Prostate Cancer. NEJM Evid. 2, (2023) is incorporated by reference herein in its entirety.
8. Erho, N. et al. Discovery and validation of a prostate cancer genomic classifier that predicts early metastasis following radical prostatectomy. PloS One 8, e66855 (2013) is incorporated herein in its entirety.
9. Feng, F. Y. et al. Association of Molecular Subtypes With Differential Outcome to Apalutamide Treatment in Nonmetastatic Castration-Resistant Prostate Cancer. JAMA Oncol. 7, 1005-1014 (2021) is incorporated by reference herein in its entirety.
10. Ross, A. E. et al. External validation of a digital pathology-based multimodal artificial intelligence architecture in the NRG/RTOG 9902 phase III trial. Eur. Urol. Oncol. In press, (2024) is incorporated by reference herein in its entirety.
11. Tward, J. D. et al. Prostate Cancer Risk Stratification in NRG Oncology Phase III Randomized Trials Using Multi-Modal Deep Learning with Digital Histopathology. Int. J. Radiat. Oncol. 114, S2 (2022) is incorporated by reference herein in its entirety.
12. Smith, M. R. et al. Blood Biomarker Landscape in Patients with High-risk Nonmetastatic Castration-Resistant Prostate Cancer Treated with Apalutamide and Androgen-Deprivation Therapy as They Progress to Metastatic Disease. Clin. Cancer Res. Off. J. Am. Assoc. Cancer Res. 27, 4539-4548 (2021) is incorporated by reference herein in its entirety.
13. Eggener, S. E. et al. Molecular Biomarkers in Localized Prostate Cancer: ASCO Guideline. J. Clin. Oncol. Off. J. Am. Soc. Clin. Oncol. 38, 1474-1494 (2020) is incorporated by reference herein in its entirety.

1.-40. (canceled)

41. A method for assessing a nonmetastatic castration-resistant prostate cancer (nmCRPC) of a subject, comprising:
(a) obtaining a dataset comprising image data and tabular data derived from said subject;
(b) processing said dataset using a trained algorithm to classify said dataset to a category among a plurality of categories, wherein said classifying comprises applying an image processing algorithm to said image data; and
(c) assessing said nmCRPC of said subject based at least in part on said category among said plurality of categories that is classified in (b).

42. The method of claim 41, wherein said trained algorithm is trained using self-supervised learning.

43. The method of claim 41, wherein said trained algorithm comprises a deep learning algorithm.

44. The method of claim 41, wherein said trained algorithm comprises a first trained algorithm processing said image data and a second trained algorithm processing said tabular data.

45. The method of claim 44, wherein said trained algorithm further comprises a third trained algorithm processing outputs of said first and second trained algorithms.

46. The method of claim 41, wherein said tabular data comprises clinical data of said subject.

47. The method of claim 46, wherein said clinical data of said subject comprises laboratory data, therapeutic interventions, or long-term outcomes.

48. The method of claim 41, wherein said image data comprises digital histopathology data.

49. The method of claim 48, wherein said histopathology data comprises images derived from a biopsy sample of said subject.

50. The method of claim 49, wherein said images are acquired via microscopy of said biopsy sample.

51. The method of claim 48, wherein said digital histopathology data is derived from said subject prior to said subject receiving a treatment.

52. The method of claim 51, wherein said treatment comprises radiotherapy (RT).

53. The method of claim 52, wherein said RT comprises pre-specified use of short-term androgen deprivation therapy (ST-ADT), long-term ADT (LT-ADT), dose escalated RT (DE-RT), or any combination thereof.

54. The method of claim 48, wherein said digital histopathology data is derived from said subject subsequent to said subject receiving a treatment.

55. The method of claim 54, wherein said treatment comprises radiotherapy (RT).

56. The method of claim 55, wherein said RT comprises pre-specified use of short-term androgen deprivation therapy (ST-ADT), long-term ADT (LT-ADT), dose escalated RT (DE-RT), or any combination thereof.

57. The method of claim 41, further comprising processing said image data using an image segmentation, image concatenation, object detection algorithm, or any combination thereof.

58. The method of claim 41, further comprising extracting a feature from said image data.

59. The method of claim 41, further comprising administering apalutamide to said subject, based at least in part on said assessing in (c).

60. The method of claim 41, further comprising predicting an endpoint of said subject, based at least in part on said assessing in (c).

61. The method of claim 60, wherein said endpoint comprises metastasis-free survival, progression-free survival, or overall survival.

62. A method for assessing a nonmetastatic castration-resistant prostate cancer (nmCRPC) of a subject, comprising:
(a) obtaining a dataset comprising at least image data derived from said subject;
(b) processing said dataset using a trained algorithm to classify said dataset to a category among a plurality of categories, wherein said classifying comprises applying an image processing algorithm to said image data, wherein said trained algorithm is trained using self-supervised learning; and
(c) assessing said nmCRPC of said subject based at least in part on said category among said plurality of categories that is classified in (b).
